(19) United States
(12) Patent Application Publication
(10) Pub. No.: US 2026/0317751 A1
(43) Pub. Date: Oct. 15, 2026

(54) SPLIT INTEINS WITH EXCEPTIONAL SPLICING ACTIVITY

(71) Applicant: THE TRUSTEES OF PRINCETON UNIVERSITY, Princeton, NJ (US)

(72) Inventors: Tom W. MUIR, Princeton, NJ (US); Adam J. STEVENS, Princeton, NJ (US); Neel H. SHAH, Berkeley, CA (US)

(21) Appl. No.: 19/170,734

(22) Filed: Apr. 4, 2025

Related U.S. Application Data
(63) Continuation of application No. 17/363,698, filed on Jun. 30, 2021, now Pat. No. 12,297,472.

(51) Int. Cl. C12N 9/00; C07K 14/00; C07K 14/32; C07K 16/28; C12N 15/62

(52) U.S. Cl. CPC C12N 9/93; C07K 14/001; C07K 14/32; C07K 16/2851; C12N 15/62; C07K 2319/21; C07K 2319/30; C07K 2319/92

(57) ABSTRACT

Embodiments of the present invention relate to inteins, split inteins, compositions comprising inteins and methods for use of these.

### CROSS-REFERENCE OF RELATED APPLICATION

This application is:
a continuation of U.S. patent application Ser. No. 17/363,698, filed Jul. 16, 2021, which is:
a continuation of U.S. patent application Ser. No. 16/073,602, filed Jul. 27, 2018, which claims priority to:
International Application No. PCT/US2017/015455 filed Jan. 27, 2017, which claims priority to
U.S. Provisional Application No. 62/288,661 filed Jan. 29, 2016.
   The entire disclosure of each prior application is hereby incorporated by reference in its entirety.

### STATEMENT OF GOVERNMENT SUPPORT

This invention was made with government support under Grant No. GM086868 awarded by the National Institutes of Health. The government has certain rights in the invention.

### INCORPORATION BY REFERENCE

The present application incorporates by reference a sequence listing, in electronic format, entitled ABG_3037-CON2_sequence_listing_Corrected, created May 15, 2026, which is 487,287 bytes in size. The information in the electronic format of the sequence listing is incorporated herein by reference in its entirety.

### BACKGROUND

### 1. Technical Field

The field of the currently claimed embodiments of the present invention relate to inteins, split inteins, compositions comprising inteins and methods for use of the like for protein engineering.

### 2. Discussion of Related Art

Protein splicing is a posttranslational auto-processing event in which an intervening protein domain called an intein excises itself from a host protein in a traceless manner such that the flanking polypeptide sequences (exteins) are ligated together via a normal peptide bond.¹ While protein splicing typically occurs spontaneously following translation of a contiguous polypeptide, some inteins exist naturally in a split form.¹ The two pieces of the split intein are expressed separately and remain inactive until encountering their complementary partner, upon which they cooperatively fold and undergo splicing in trans. This activity has been harnessed in a host of protein engineering methods that provide control over the structure and activity of proteins both in vitro and in vivo.¹ The first two split inteins to be characterized, from the cyanobacteria Synechocystis species PCC6803 (Ssp) and Nostoc punctiforme PCC73102 (Npu), are orthologs naturally found inserted in the alpha subunit of DNA Polymerase III (DnaE).²⁻⁴ Npu is especially notable due its remarkably fast rate of protein trans-splicing (PTS) (t_{1/2}=50 s at 30° C.).⁵ This half-life is significantly shorter than that of Ssp (t_{1/2}=80 min at 30° C.),⁵ an attribute that has expanded the range of applications open to PTS.¹

Despite the ongoing discovery of new fast inteins,^{6,7} little is known about what separates them from their slower homologues. Such an understanding should help identify new inteins that are likely to splice rapidly and potentially allow for the engineering of split inteins with superior PTS properties.

### SUMMARY

Embodiments of the invention include a split intein N-fragment including an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL.

Embodiments of the invention include a split intein N-fragment including an amino acid sequence, wherein said amino acid sequence comprises an amino acid sequence of at least 80%, 85%, 90%, 95%, 98%, 99%, or 100% sequence identity to

(SEQ ID NO: 2)
CLSYDTEILTVEYGFLPIGKIVEERIECTVYTVDKNGPVYTQPIAQWHN
RGEQEVFEYCLEDGSIIRATKDHKFMTTDGQMLPIDEIFERGLDLKQVD
GLP.

Embodiments of the invention include a split intein C-fragment including an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN.

Embodiments of the invention include a split intein C-fragment including an amino acid sequence, wherein said amino acid sequence of said C-fragment comprises an amino acid sequence of at least 80%, 85%, 90%, 95%, 98%, 99%, or 100% sequence identity to

(SEQ ID NO: 4)
MVKIISRKSLGTQNVYDIGVEKDHNFLLKNGLVASN.

Embodiments of the invention include a split intein C-fragment including an amino acid sequence, wherein said amino acid sequence of said C-fragment comprises an amino acid sequence of at least 80%, 85%, 90%, 95%, 98%, 99%, or 100% sequence identity to

(SEQ ID NO: 389)
VKIISRKSLGTQNVYDIGVGEPHNFLLKNGLVASN.

Embodiments of the invention include a composition including a split intein N-fragment comprising an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP.

and a split intein C-fragment comprising an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

Embodiments of the invention include a nucleotide plasmid including a nucleotide sequence encoding for a split intein N-fragment comprising an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP.

Embodiments of the invention include a nucleotide plasmid comprising a nucleotide sequence encoding for a split intein C-fragment including an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

Embodiments of the invention include a method for splicing two complexes including the following: contacting a first complex comprising a first compound and a split intein N-fragment with a second complex comprising a second compound and a split intein C-fragment, with contacting performed under conditions that permit binding of the split intein N-fragment to the split intein C-fragment to form an intein intermediate; and reacting the intein intermediate to form a conjugate of the first compound with the second compound. The split intein N-fragment includes an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP,

and the split intein C-fragment comprises an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

Embodiments of the invention include a method including the following: contacting a first complex comprising a first compound and a split intein N-fragment with a second complex comprising a second compound and a split intein C-fragment, with the contacting performed under conditions that permit binding of the split intein N-fragment to the split intein C-fragment to form an intein intermediate; and reacting the intein intermediate with a nucleophile to form a conjugate of the first compound with the nucleophile. The split intein N-fragment includes an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP,

the split intein C-fragment includes an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

In some embodiments, the compound, first compound, or second compound is or includes a peptide or a polypeptide. In some embodiments, the compound, first compound, or second compound is or includes an antibody, antibody chain, or antibody heavy chain. In some embodiments, the compound, first compound, or second compound is or includes a peptide, oligonucleotide, drug, or cytotoxic molecule.

Embodiments of the invention include an intein comprising an amino acid sequence of at least 80%, 85%, 90%, 95%, 98%, 99%, or 100% sequence identity to

(SEQ ID NO: 390)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLRQVDGL
PVKIISRKSLGTQNVYDIGVEKDHNFLLKNGLVASN.

Embodiments of the invention include a kit for splicing two complexes together including the following: a split intein N-fragment including an amino acid sequence of at least 80%, 85% 90% 95% 98% 99% or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P;

a split intein C-fragment comprising an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

reagent(s) for permitting the binding of the split intein N-fragment to the split intein C-fragment to form an intein intermediate; and a nucleophilic agent.

Embodiments of the invention include a method for generating a synthetic consensus intein peptide sequence including the following: generating a population of a plurality of homologous intein peptide sequences; identifying amino acids associated with fast splicing within the population of a plurality of homologous intein peptide sequences; generating a subpopulation of a second plurality of homologous intein peptide sequences, with the second plurality of homologous intein peptide sequences including amino acids associated with fast splicing; creating an alignment of at least three peptide sequences of the subpopulation; determining a most frequently occurring amino acid residue at each position of the at least three peptide sequences; and generating a synthetic consensus intein peptide sequence based on the most frequently occurring amino acid residue at each position of the at least three peptide sequences.

Embodiments of the invention include a method including the following: fusing a first nucleotide sequence encoding an amino acid sequence of a first intein fragment (split intein N-fragment) including with a second nucleotide sequence encoding an amino acid sequence of a second intein fragment (split intein C-fragment), so that the fusion of the first nucleotide sequence and the second nucleotide sequence codes for a contiguous intein. The split intein N-fragment includes an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P,

and the split intein C-fragment includes an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

Embodiments of the invention include a method including the following: fusing a first nucleotide sequence encoding an amino acid sequence of a first intein fragment (split intein N-fragment) including CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1) with a second nucleotide sequence encoding an amino acid sequence of a second intein fragment (split intein C-fragment) including VKIISRKSLGTQNVYDIGVEKDHNFLLKNGLVASN (SEQ ID NO: 3), so that the fusion of the first nucleotide sequence and the second nucleotide sequence codes for a contiguous intein.

Embodiments of the invention include a gene fusion including the following: a first nucleotide sequence encoding an amino acid sequence of a first intein fragment (split intein N-fragment) with a second nucleotide sequence encoding an amino acid sequence of a second intein fragment (split intein C-fragment). The split intein N-fragment includes an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL
or
(SEQ ID NO: 2)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P,

and the split intein C-fragment includes an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN,
(SEQ ID NO: 4)
MVKIISRKSLGTQNVYDIGVEKDHNFLLKNGLVASN,
or
(SEQ ID NO: 389)
VKIISRKSLGTQNVYDIGVGEPHNFLLKNGLVASN.

Embodiments of the invention include a gene fusion including the following: a first nucleotide sequence encoding an amino acid sequence of a first intein fragment (split intein N-fragment) including CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1) fused with a second nucleotide sequence encoding an amino acid sequence of a second intein fragment (split intein C-fragment) including VKIISRKSLGTQNVYDIGVEKDHNFLLKNGLVASN (SEQ ID NO: 3).

Embodiments of the invention include a complex (e.g., a fusion protein) comprising a split intein N-fragment and a compound. For example, the compound can be or include a peptide, a polypeptide or an antibody chain, such as an antibody heavy chain. For example, the compound can include a peptide, oligonucleotide, drug, or cytotoxic molecule. For example, the compound can be a 1,2-amino thiol or a 1,2-amino alcohol bonded to a peptide, oligonucleotide, drug, or cytotoxic molecule. The split intein N-fragment includes an amino acid sequence of at least 80%, 85%, 90%, 95%, 98%, 99%, or 100% sequence identity to CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1) or an amino acid sequence of at least 80%, 85%, 90%, 95%, 98%, 99%, or 100% sequence identity to

(SEQ ID NO: 2)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P.

Embodiments of the invention include a complex (e.g., a fusion protein) comprising a split intein C-fragment and a compound. For example, the compound can be or include a dendrimer, peptide or polypeptide. For example, the compound can include a peptide, an oligonucleotide, a drug, or a cytotoxic molecule. For example, the compound can be a 1,2-amino thiol or a 1,2-amino alcohol bonded to a peptide, oligonucleotide, drug, or cytotoxic molecule. The split intein C-fragment includes an amino acid sequence of at least 80%, 85%, 90%, 95%, 98%, 99%, or 100% sequence identity to VKIISRKSLGTQNVYDIGVEKDHNFLLKNGLVASN (SEQ ID NO: 3), an amino acid sequence of at least 80%, 85%, 90%, 95%, 98%, 99%, or 100% sequence identity to MVKIISRKSLGTQNVYDIGVEKDHNFLLKNGLVASN (SEQ ID NO: 4), or an amino acid sequence of at least 80%, 85%, 90%, 95%, 98%, 99%, or 100% sequence identity to VKIISRKSLGTQNVYDIGVGEPHNFLLKNGLVASN (SEQ ID NO: 389). The dendrimer can be a compound having the structure

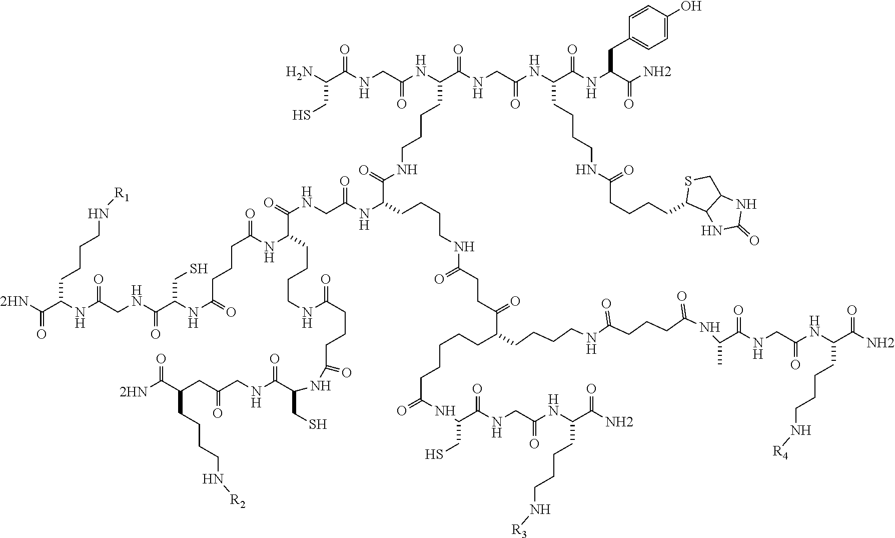

wherein R1, R2, R3, and R4 are each (independently) hydrogen (H) or a cargo molecule (the cargo molecules on R1, R2, R3, and R4 can be different from each other). R1, R2, R3, and R4 can each be a dye molecule. For example, R1, R2, R3, and R4 can each be a fluorescein derivative having the structure

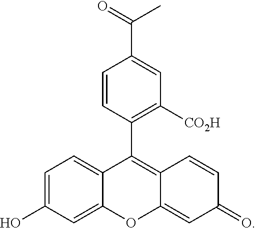 [SMILES: CC(=O)c1ccc(-c2c3ccc(=O)cc-3oc3cc(O)ccc23)c(C(=O)O)c1]

Embodiments of the invention include a complex of the structure

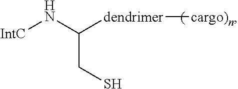

with IntC being a split intein C-fragment and n being from 0 to 8.

Embodiments of the invention include a complex of the structure

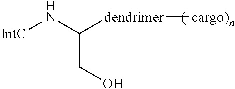

with IntC being a split intein C-fragment and n being from 0 to 8.

Embodiments of the invention include a complex of the structure

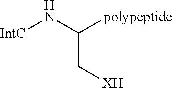

with IntC being a split intein C-fragment and X being sulfur (S) or oxygen (O). The split intein C-fragment comprises an amino acid sequence of at least 80%, 85%, 90%, 95%, 98%, 99%, or 100% sequence identity to VKIISRKSLGTQNVYDIGVEKDHNFLLKNGLVASN (SEQ ID NO: 3), an amino acid sequence of at least 80%, 85%, 90%, 95%, 98%, 99%, or 100% sequence identity to MVKIISRKSLGTQNVYDIGVEKDHNFLLKNGLVASN (SEQ ID NO: 4), or an amino acid sequence of at least 80%, 85%, 90%, 95%, 98%, 99%, or 100% sequence identity to VKIISRKSLGTQNVYDIGVGEPHNFLLKNGLVASN (SEQ ID NO: 389).

Embodiments of the invention include a contiguous intein that can be used, for example, in traditional semi-synthesis applications such as Expressed Protein ligation.

### BRIEF DESCRIPTION OF THE DRAWINGS

FIG. 1 shows an alignment and a computer-generated model of the design of the Cfa split intein according to an embodiment of the invention, where, in Panel a of FIG. 1,
NpuN corresponds to SEQ ID NO: 5,
NpuC corresponds to SEQ ID NO: 6,
CfaN corresponds to SEQ ID NO: 2, and
CfaC corresponds to SEQ ID NO: 3;

FIG. 2 shows graphs showing the characterization of the Cfa intein according to an embodiment of the invention;

FIGS. 3A to 3E show expression and modification of a mouse monoclonal antibody using the Cfa intein according to an embodiment of the invention;

FIG. 4 shows the identification of second shell ‘accelerator’ residues important for rapid protein trans-splicing according to an embodiment of the invention;

FIG. 5 shows kinetic analysis of Batch 2 mutations and computer generated models according to an embodiment of the invention;

FIG. 6 shows an analysis of Batch 1 mutations and computer generated models according to an embodiment of the invention;

FIGS. 7A.1 to 7A.3, 7B.1 to 7B.3, 7C.1 to 7C.3, 7D.1 to 7D.3, 7E.1 to 7E.3, 7F.1 to 7F.3, 7G.1 to 7G.3, and 7H.1 to 7H.3 show an alignment and refinement of the DnaE intein family according to an embodiment of the invention, where:
FIGS. 7A.1, 7B.1, 7C.1, 7D.1, 7E.1, 7F.1, 7G.1, and 7H.1 correspond to amino acids 1-50 of the indicated sequences of split intein N-fragments,
FIGS. 7A.2, 7B.2, 7C.2, 7D.2, 7E.2, 7F.2, 7G.2, and 7H.2 correspond to the amino acids 51-100 of the indicated sequences of split intein N-fragments, and
FIGS. 7A.3, 7B.3, 7C.3, 7D.3, 7E.3, 7F.3, 7G.3, and 7H.3 correspond to the amino acids 101-102 of the indicated sequences of split intein N-fragments, followed by the sequences of the split intein C-fragments,

FIGS. 7A.1 and 7A2 correspond to the sequences of SEQ ID NO: 5, 7, 9, 11, 13, 15, 17, 19, 21, 23, 25, 27, 29, 31, 33, 35, 37, 39, 41, 43, 45 and 47;

FIG. 7A3 corresponds to the last two amino acids of the sequences of FIGS. 7A.1 and 7A.2 followed by the sequences of SEQ ID NO: 6, 8, 10, 12, 14, 16, 18, 20, 22, 24, 26, 28, 30, 32, 34, 36, 38, 40, 42, 44, 46 and 48;

FIGS. 7B.1 and 7B.2 correspond to the sequences of SEQ ID NO: 49, 51, 53, 55, 57, 59, 61, 63, 65, 67, 69, 71, 73, 75, 77, 79, 81, 83, 85, 87, 89 and 91;

FIG. 7B.3 corresponds to the last two amino acids of the sequences of FIGS. 7B.1 and 7B.2 followed by the sequences of SEQ ID NO: 50, 52, 54, 56, 58, 60, 62, 64, 66, 68, 70, 72, 74, 76, 78, 80, 82, 84, 86, 88, 90 and 92;

FIGS. 7C.1 and 7C.2 correspond to the sequences of SEQ ID NO: 93, 95, 97, 99, 101, 103, 105, 107, 109, 111, 113, 115, 117, 119, 121, 123, 125, 127, 129, 131, 133 and 135;

FIG. 7C.3 corresponds to the last two amino acids of the sequences of FIGS. 7C.1 and 7C.2 followed by the sequences of SEQ ID NO: 94, 96, 98, 100, 102, 104, 106, 108, 110, 112, 114, 116, 118, 120, 122, 124, 126, 128, 130, 132, 134 and 136;

FIGS. 7D.1 and 7D.2 correspond to the sequences of SEQ ID NO: 137, 139, 141, 143, 145, 147, 149, 151, 153, 155, 157, 159, 161, 163, 165, 167, 169, 171, 173, 175, 177 and 179;

FIG. 7D.3 corresponds to the last two amino acids of the sequences of FIGS. 7D.1 and 7D.2 followed by the sequences of SEQ ID NO: 138, 140, 142, 144, 146, 148, 150, 152, 154, 156, 158, 160, 162, 164, 166, 168, 170, 172, 174, 176, 178 and 180;

FIGS. 7E.1 and 7E.2 correspond to the sequences of SEQ ID NO: 181, 183, 185, 187, 189, 191, 193, 195, 197, 199, 201, 203, 205, 207, 209, 211 and 213;

FIG. 7E.3 corresponds to the last two amino acids of the sequences of FIGS. 7E.1 and 7E.2 followed by the sequences of SEQ ID NO: 182, 184, 186, 188, 190, 192, 194, 196, 198, 200, 202, 204, 206, 208, 210, 212 and 214;

FIGS. 7F.1 and 7F.2 correspond to the sequences of SEQ ID NO: 215, 217, 219, 221, 223, 225, 227, 229, 231, 233, 235, 237, 239, 241, 243, 245, 247, 249, 251, 253, 255, 257, 259 and 261;

FIG. 7F.3 corresponds to the last two amino acids of the sequences of FIGS. 7F.1 and 7F.2 followed by the sequences of SEQ ID NO: 216, 218, 220, 222, 224, 226, 228, 230, 232, 234, 236, 238, 240, 242, 244, 246, 248, 250, 252, 254, 256, 258, 260 and 262;

FIGS. 7G.1 and 7G.2 correspond to the sequences of SEQ ID NO: 263, 265, 267, 269, 271, 273, 275, 277, 279, 281, 283, 285, 287, 289, 291, 293, 295, 297, 299, 301, 303, 305, 307, 309 and 311;

FIG. 7G.3 corresponds to the last two amino acids of the sequences of FIGS. 7G.1 and 7G.2 followed by the sequences of SEQ ID NO: 264, 266, 268, 270, 272, 274, 276, 278, 280, 282, 284, 286, 288, 290, 292, 294, 296, 298, 300, 302, 304, 306, 308, 310 and 312;

FIGS. 7H.1 and 7H.2 correspond to the sequences of SEQ ID NO: 313, 315, 317, 319, 321, 323, 325, 327, 329, 331, 333, 335, 337, 339, 341, 343, 345, 347, 349, 351, 353, 355, 357 and 359; and

FIG. 7H.3 corresponds to the last two amino acids of the sequences of FIGS. 7H.1 and 7H.2 followed by the sequences of SEQ ID NO: 314, 316, 318, 320, 322, 324, 326, 328, 330, 332, 334, 336, 338, 340, 342, 344, 346, 348, 350, 352, 354, 356, 358 and 360;

FIG. 8 is an image of an SDS-PAGE analysis of test expression of His₆-SUMO-Npu^{N} and His₆-SUMO-Cfa^{N} according to an embodiment of the invention;

FIG. 9 shows a schematic and graph showing increased promiscuity of Cfa_{GEP} according to an embodiment of the invention;

FIG. 10 shows graphs and schematics showing cyclization of eGFP in E. coli with variable residues according to an embodiment of the invention; and

FIG. 11 shows a table illustrating several complexes and compounds according to an embodiment of the invention.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are hereby incorporated by reference in their entirety as if each had been individually incorporated.

Embodiments of the invention include a split intein N-fragment comprising an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL.

Embodiments of the invention include a split intein N-fragment comprising an amino acid sequence, wherein said amino acid sequence comprises an amino acid sequence of at least 80%, 85%, 90%, 95%, 98%, 99%, or 100% sequence identity to

(SEQ ID NO: 2)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P.

Embodiments of the invention include a split intein C-fragment comprising an amino acid sequence of at least 80%, 85%, 90%, 95%, 98%, 99%, or 100% sequence identity to

(SEQ ID NO: 3)
VKIISRKSLGTQNVYDIGVEKDHNFLLKNGLVASN.

Embodiments of the invention include a split intein C-fragment comprising an amino acid sequence, wherein said amino acid sequence of said C-fragment comprises an amino acid sequence of at least 80%, 85%, 90%, 95%, 98%, 99%, or 100% sequence identity to MVKIISRKSLGTQNVYDIGVEKDHNFLLKNGLVASN (SEQ ID NO: 4).

Embodiments of the invention include a composition comprising the following: a split intein N-fragment comprising an amino acid sequence of at least 80%, 85%, 90%, 95%, 98%, 99%, or 100% sequence identity to CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1); and a split intein C-fragment comprising an amino acid sequence of at least 80%, 85%, 90%, 95%, 98%, 99%, or 100% sequence identity to VKIISRKSLGTQNVYDIGVEKDHNFLLKNGLVASN (SEQ ID NO: 3).

Embodiments of the invention include a nucleotide plasmid comprising a nucleotide sequence encoding for a split intein N-fragment comprising an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL.

Embodiments of the invention include a nucleotide plasmid comprising a nucleotide sequence encoding for a split intein C-fragment comprising an amino acid sequence of at least 80%, 85%, 90%, 95%, 98%, 99%, or 100% sequence identity to

(SEQ ID NO: 1)
CLSYDTEILTVEYGFLPIGKIVEERIECTVYTVDKNGFVYTQPIAQWHNR
GEQEVFEYCLEDGSIIRATKDHKFMTTDGQMLPIDEIFERGL.

Embodiments of the invention include a method for splicing two complexes comprising: contacting a first complex comprising a first compound and a split intein N-fragment and a second complex comprising a second compound and a split intein C-fragment, wherein contacting is performed under conditions that permit binding of the split intein N-fragment to the split intein C-fragment to form an intein intermediate; and reacting the intein intermediate to form a conjugate of the first compound with the second compound, wherein said split intein N-fragment comprises an amino acid sequence of at least 80%, 85%, 90%, 95%, 98%, 99%, or 100% sequence identity to CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1), and wherein said split intein C-fragment comprises an amino acid sequence of at least 80%, 85%, 90%, 95%, 98%, 99%, or 100% sequence identity to VKIISRKSLGTQNVYDIGVEKDHNFLLKNGLVASN (SEQ ID NO: 3). In some embodiments, reacting the intein intermediate comprises contacting the intein intermediate with a nucleophile. In some embodiments, said first compound is a polypeptide. In some embodiments, said first compound is an antibody.

Embodiments of the invention include an intein comprising an amino acid sequence of at least 80%, 85%, 90%, 95%, 98%, 99%, or 100% sequence identity to

(SEQ ID NO: 390)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLRQVDGL
PVKIISRKSLGTQNVYDIGVEKDHNFLLKNGLVASN.

Embodiments of the invention include a kit for splicing two complexes together comprising the following: a split intein N-fragment comprising an amino acid sequence of at least 80%, 85%, 90%, 95%, 98%, 99%, or 100% sequence identity to CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1); a split intein C-fragment comprising an amino acid sequence of at least 80%, 85%, 90%, 95%, 98%, 99%, or 100% sequence identity to VKIISRKSLGTQNVYDIGVEKDHNFLLKNGLVASN (SEQ ID NO: 3); reagents for permitting the binding of the split intein N-fragment to the split intein C-fragment to form an intein intermediate; and a nucleophilic agent.

Embodiments of the invention include a method for generating a synthetic consensus intein peptide sequence comprising: generating a population of a plurality of homologous intein peptide sequences; identifying amino acids associated with fast splicing within said population of a plurality of homologous intein peptide sequences; generating a subpopulation of a second plurality of homologous intein peptide sequences, wherein said second plurality of homologous intein peptide sequences comprise amino acids associated with fast splicing; creating an alignment of at least three peptide sequences of said subpopulation; determining a most frequently occurring amino acid residue at each position of said at least three peptide sequences; and generating a synthetic consensus intein peptide sequence based on said most frequently occurring amino acid residue at each position of said at least three peptide sequences.

Embodiments of the invention include a method comprising: fusing a first nucleotide sequence encoding an amino acid sequence of a first intein fragment comprising CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1) with a second nucleotide sequence encoding an amino acid sequence of a second intein fragment comprising VKIISRKSLGTQNVYDIGVEKDHNFLLKNGLVASN (SEQ ID NO: 3), so that the fusion of the first nucleotide sequence and second nucleotide sequence codes for a contiguous intein.

Embodiments of the invention include a gene fusion comprising a first nucleotide sequence encoding an amino acid sequence of a first intein fragment comprising CLSYDTEILTVEYGFLPIGKIVEERIECTVYTVDKNGFVYTQPIAQWHNRGEQEVFEYCLED GSIIRATKDHKFMTTDGQMLPIDEIFERGL (SEQ ID NO: 1) fused with a second nucleotide sequence encoding an amino acid sequence of a second intein fragment comprising VKIISRKSLGTQNVYDIGVEKDHNFLLKNGLVASN (SEQ ID NO: 3).

Embodiments of the invention include a contiguous intein that can be used, for example, in traditional semi-synthesis applications such as Expressed Protein ligation.

In some embodiments, the various intein fragments described are linked, fused, chemically bonded, complexed or coupled by conventional methods known in the art to polymers, peptides, polypeptides, oligopeptides, small molecules, nucleotides, polynucleotides, oligonucleotides, drugs, cytotoxic molecules or combinations thereof.

### Example 1

In some embodiments, the basis of rapid protein splicing through a comparative study of the first two characterized split inteins, Npu and Ssp was investigated. The substantial difference in splicing rate between these two proteins is especially puzzling given their highly similar sequences (63% identity) and near superimposable active site structures. Previous mutagenesis studies on Npu and Ssp suggest that the difference in activity between the two is likely due to the combined effects of several residues, rather than a single site.^{6,8} However, it remains unclear just how many residues are responsible for the fast versus slow reaction rates and by extension, whether these ‘accelerator’ residues contribute equally to the individual chemical steps in the overall protein splicing process. Consequently, we began our study by exploring these questions, in the hope that this would provide a starting point for developing an improved PTS system.

The high level of conservation within the active sites of Npu and Ssp suggests that more distal amino acid differences account for the disparity in splicing rate between the two. Thus, attention was focused on ‘second shell’ residues, those directly adjacent to the active site. To simplify this analysis, a batch mutagenesis strategy was employed in conjunction with a previously reported in vitro PTS assay.⁵ This assay uses split intein constructs with short native extein sequences and allows the rates of branched intermediate formation (k₁,k₂) and its resolution to final splice products (k₃) to be determined using a three state kinetic model.

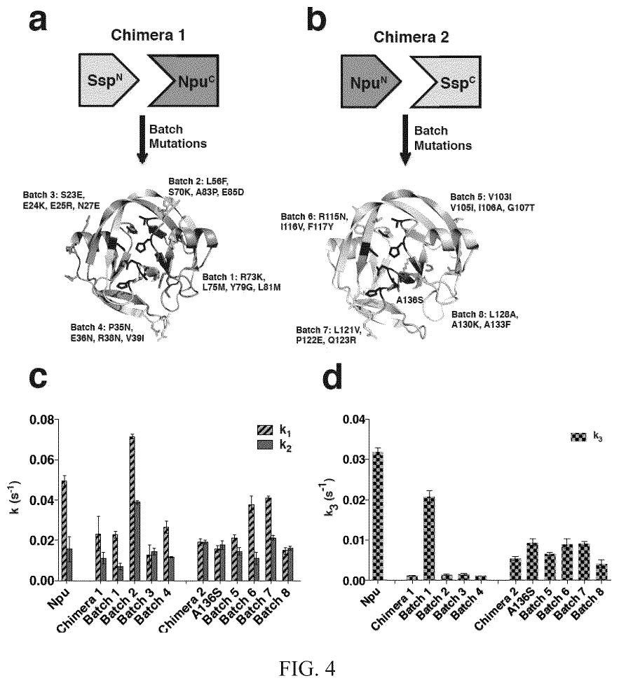

The known cross-reactivity of Npu and Ssp intein fragments served as a convenient platform on which to assess which half of the split intein contributes most significantly to the difference in activity.³ Both the Ssp^{N}-Npu^{C} (chimera 1) and Npu^{N}-Ssp^{C} (chimera 2) chimeras show a decrease in the rates of branch formation and resolution compared to that of native Npu (FIG. 4C, 4D). This indicates that residues on both the N- and C-intein fragments of Npu and Ssp contribute to the difference in their splicing rate. Next, four groups of second shell positions on each of these chimeras were chosen based on their proximity to the active site, and the corresponding Ssp residues were mutated to those in Npu (FIGS. 4A and 4B). From the chimera 1 mutants, Batch 2 (L56F, S70K, A83P, E85D) completely restored branch formation activity to that of native Npu (FIG. 4C), while Batch 1 (R73K, L75M, Y79G, L81M) restored the majority of branch resolution activity (FIG. 4D). The effects of mutations on the chimera 2 background were more prosaic, with no single batch able to restore splicing activity to that of native Npu (FIGS. 4C and 4D). Lastly, the A136S mutation on Ssp^{C} has previously been shown to accelerate protein splicing and was examined separately.⁸ This A136S mutation increases the rate of branch resolution two fold, but has no impact on branch formation (FIGS. 4C and 4D).

FIG. 4 shows the identification of second shell ‘accelerator’ residues important for rapid protein trans-splicing according to an embodiment of the invention. In Panels A and B, design of second shell batch mutants on chimera 1 (Ssp^{N}-Npu^{C}) and chimera 2 (Npu^{N}-Ssp^{C}) is shown. In each case, the location of the mutants (rendered as sticks) is shown using the crystal structure of Npu (pdb=4kl5). Catalytic residues are shown in black (rendered as sticks). Panel C shows forward (k₁) and reverse (k₂) rates of branched intermediate formation from starting materials for the various constructs described in this study (error=SD (n=3)). Panel D shows the rate of branch resolution (k₃) of the various constructs (error=SD (n=3)).

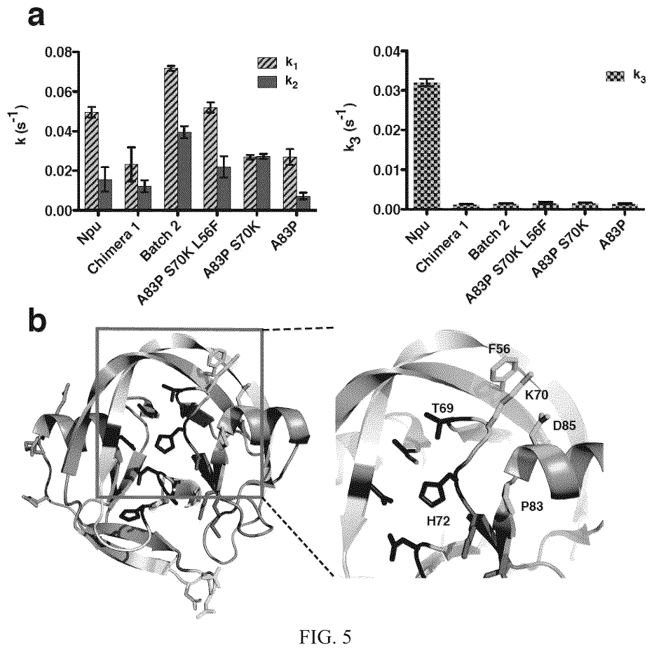

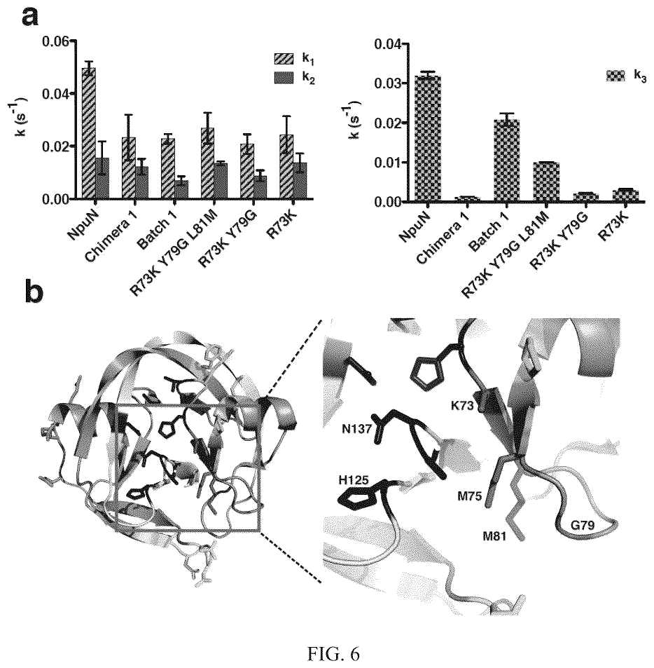

Next the individual contributions of residues within batch mutants 1 and 2 was investigated, since these had the most profound effect on splicing activity. For Batch 2, further mutagenesis shows that the interaction between F56, K70, and D85 is likely responsible for the increased rate of branch formation in Npu^{N} (FIG. 5A). Structural evidence supports this data, as K70 is a part of the highly conserved TXXH block B loop in Npu^{N} (residues 69-72) that catalyzes the initial N-to-S acyl shift in protein splicing.⁹ Thus, the position and dynamics of K70 (packed against F56 and D85) should directly impact the catalytic residues T69 and H72 (FIG. 5B).¹⁰⁻¹² From Batch 1, K73, M75, and M81 are responsible for the faster rate of branch resolution in Npu^{N} (FIG. 6A). These residues pack around the terminal asparagine of the C-intein, which must undergo succinimide formation in the final step of protein splicing (FIG. 6B). Taken together, the mutagenesis data points to the key role that second shell ‘accelerator’ residues play in tuning the activity of split inteins.

FIG. 5 shows kinetic analysis of Batch 2 mutations and computer generated models according to an embodiment of the invention. Panel A shows the equilibrium rates of branch formation (k1, k2) and rates of branch resolution (k3) for the single (A83P), double (A83P, S70K), and triple (L56F, S70K, A83P) point mutants of Ssp^{N} that comprise Batch 2 (L56F, S70K, A83P, E85D) (error=SD (n=3)). Panel B shows a zoom view of Batch 2 (sticks next to labels F56, K70, P83, and D85) in the Npu active site (pdb=4kl5). Catalytic residues are rendered as black sticks.

FIG. 6 shows an analysis of Batch 1 mutations and computer generated models according to an embodiment of the invention. Panel A shows the equilibrium rates of branch formation (k1, k2) and rates of branch resolution (k3) for the single (R73K), double (R73K, Y79G), and triple (R73K, Y79G, L81M) point mutants comprising Batch 1 (error=SD (n=3)). Panel B shows a zoom view of Batch 1 (sticks next to labels K73, M75, G79, and M81) in the Npu structure (pdb=4kl5). Catalytic residues are rendered as black sticks.

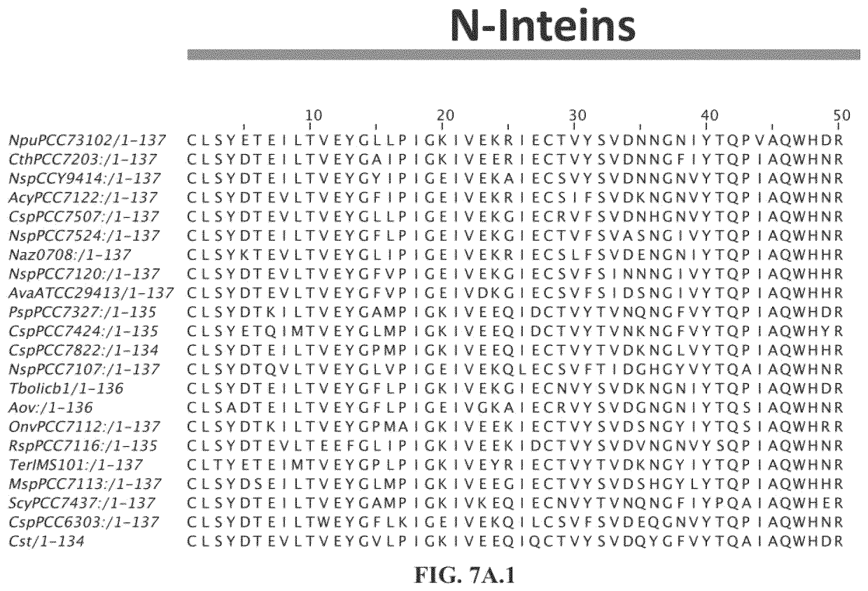

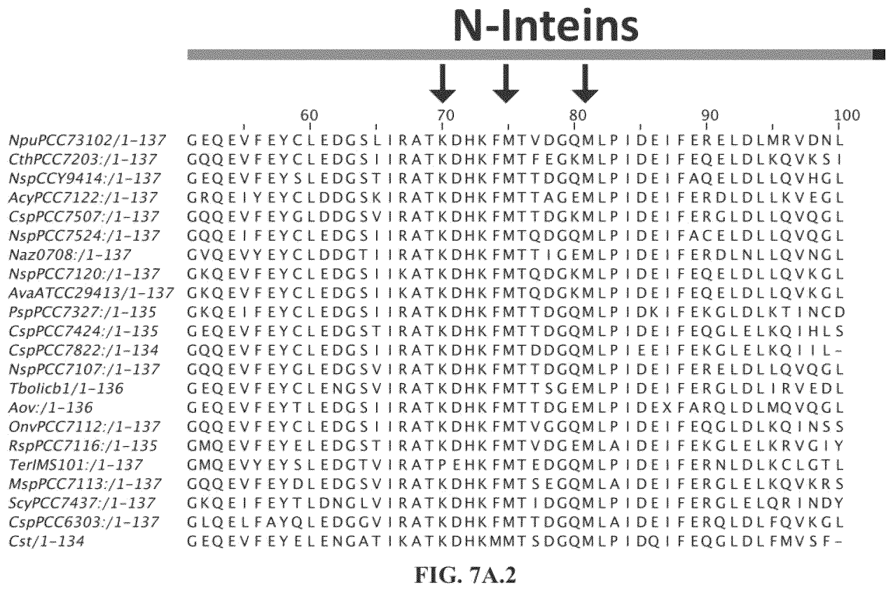

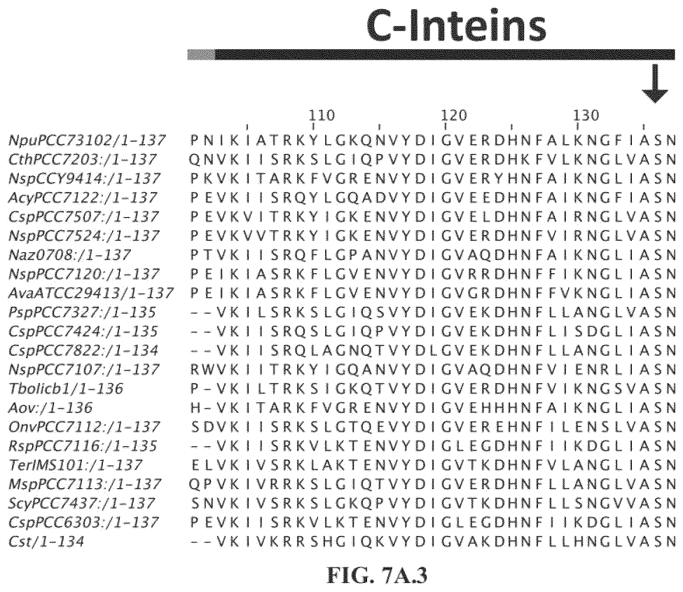

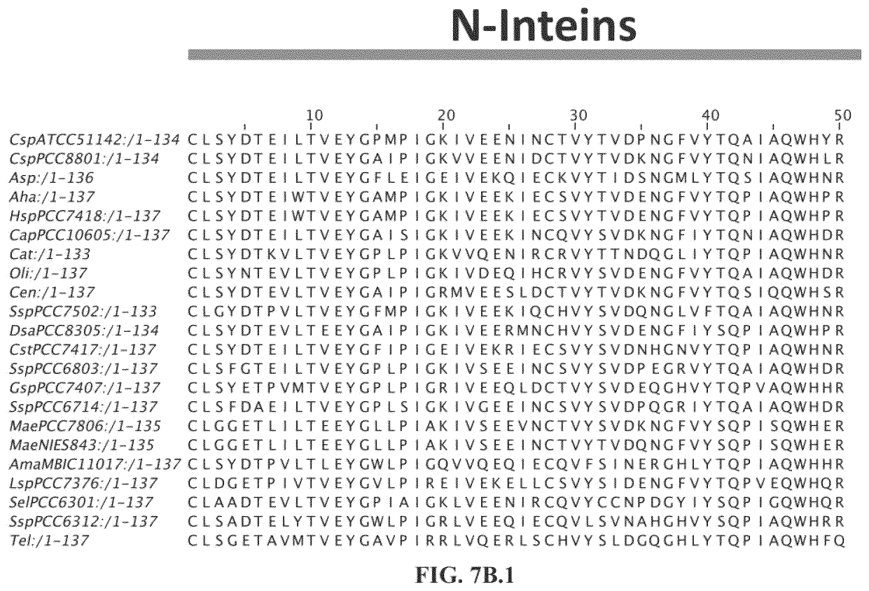

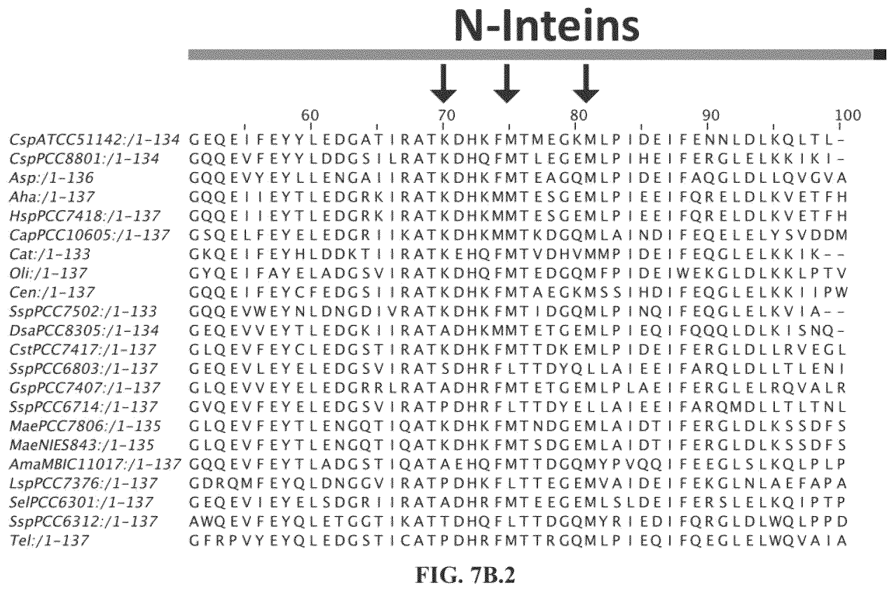

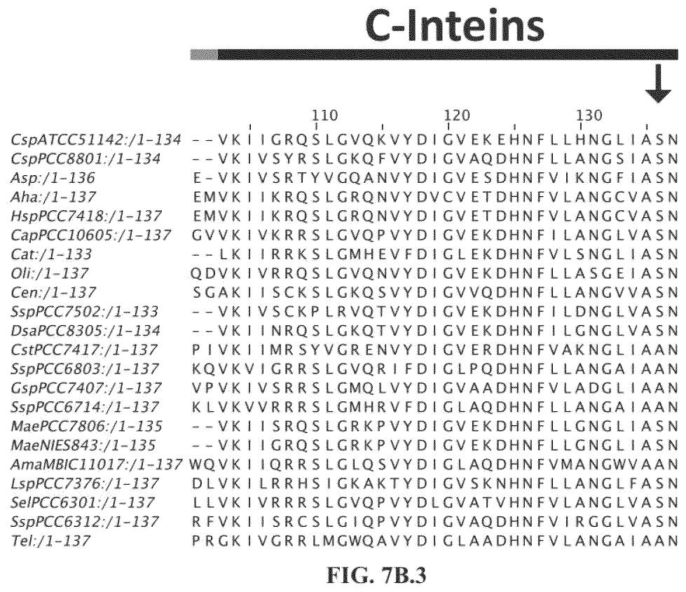

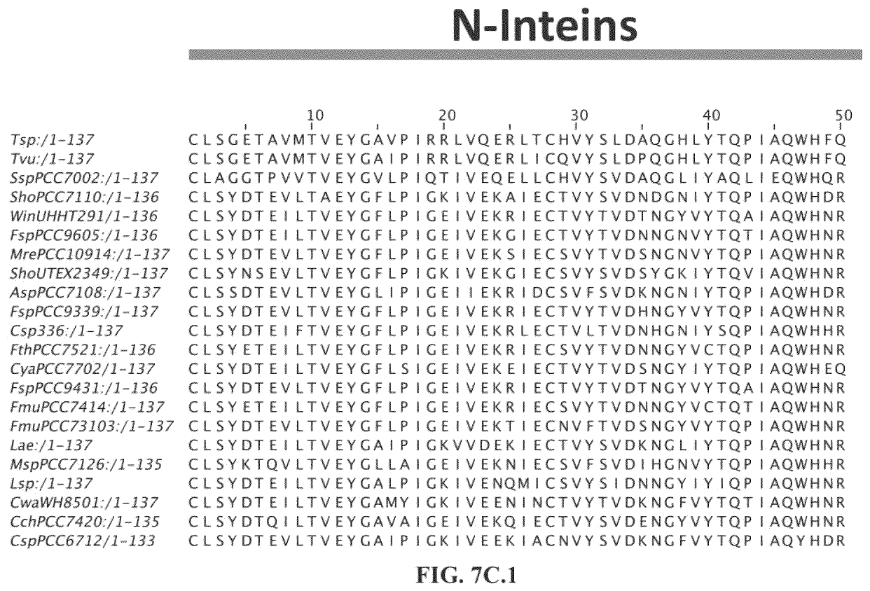

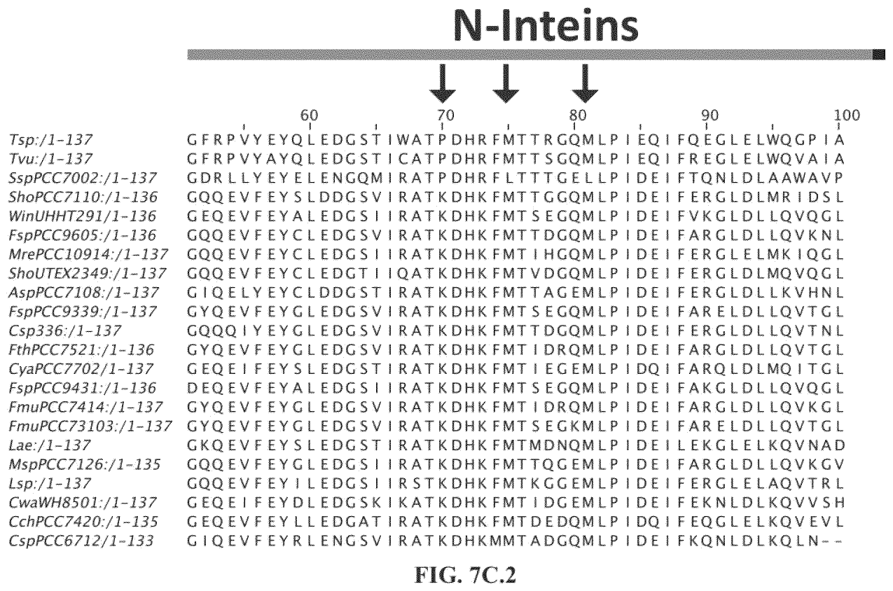

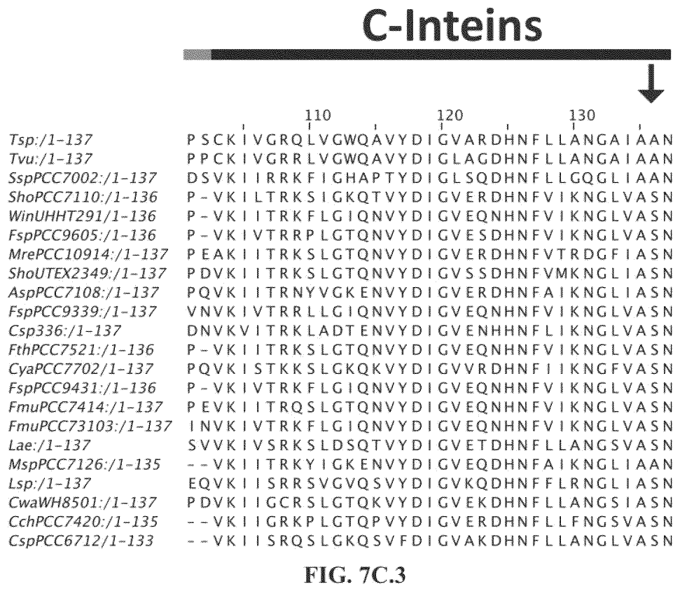

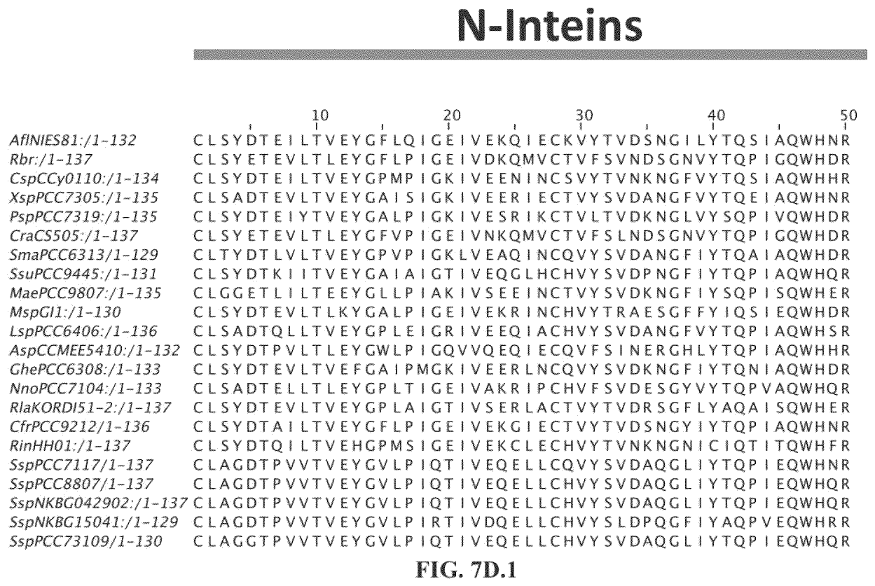

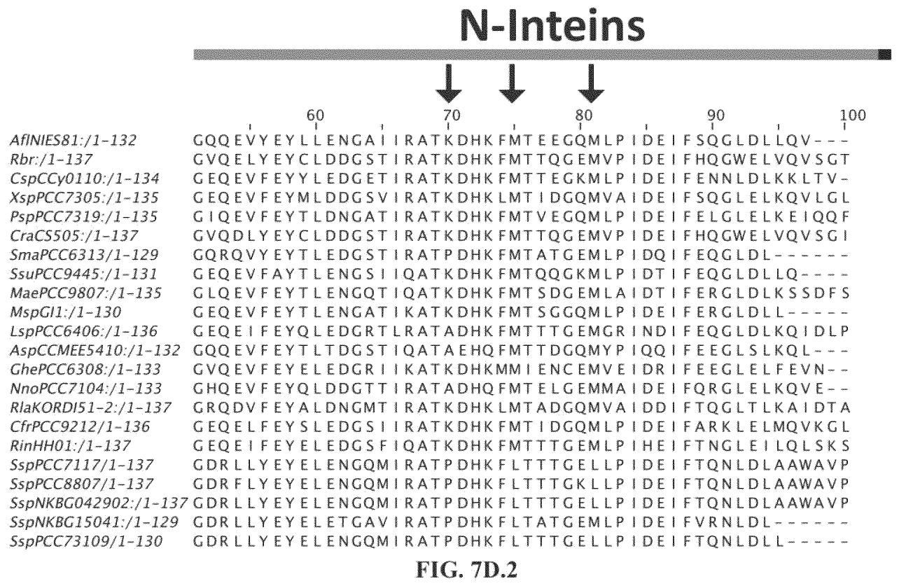

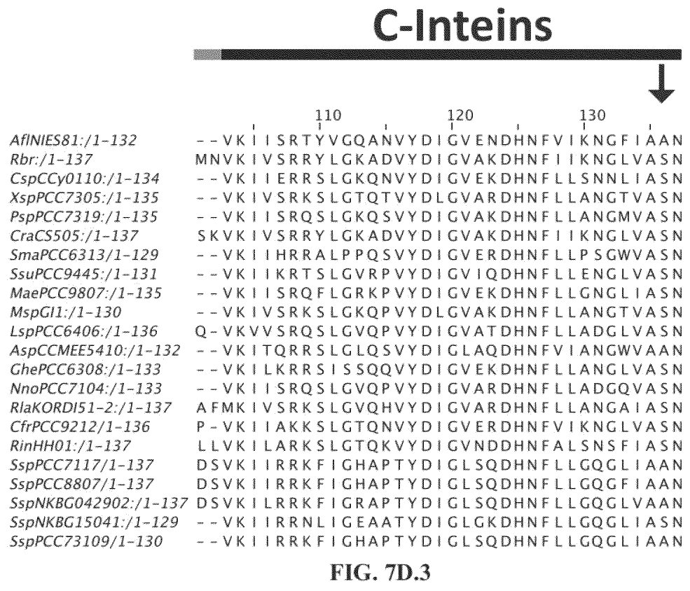

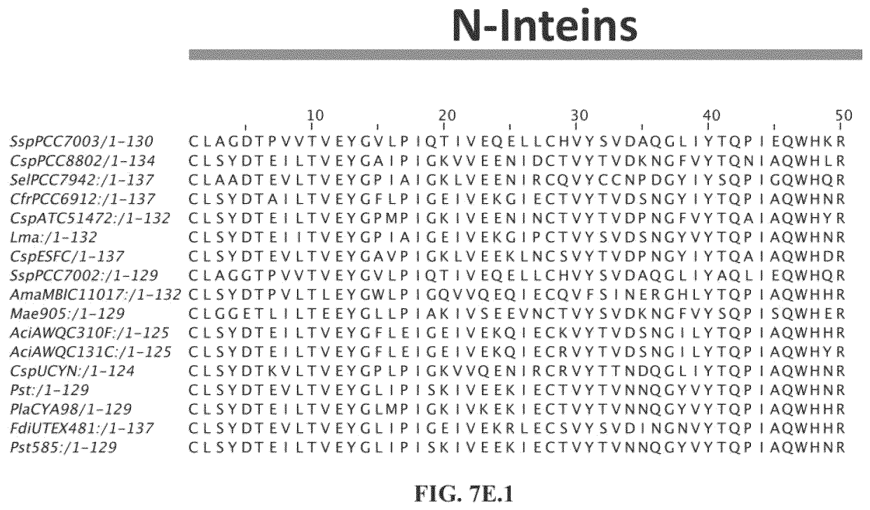

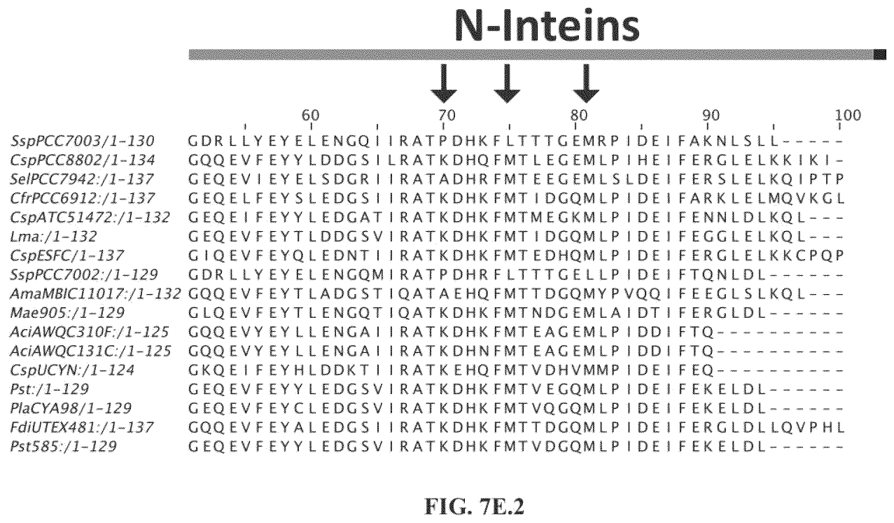

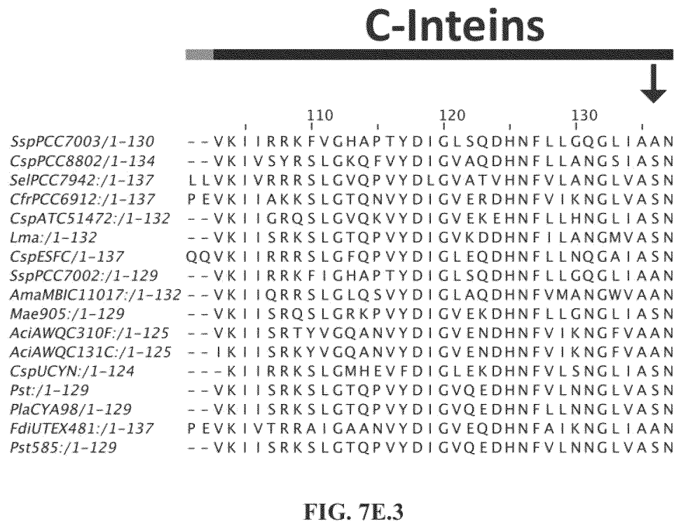

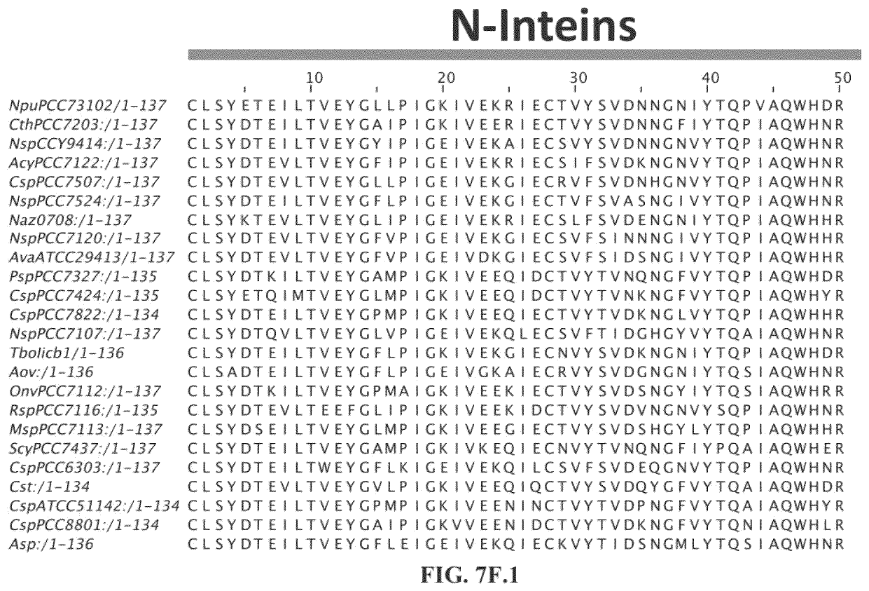

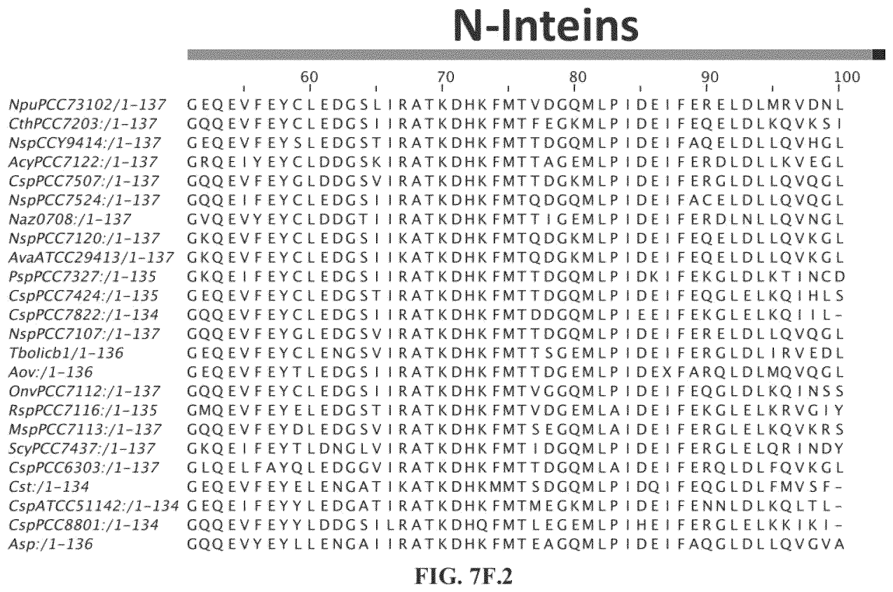

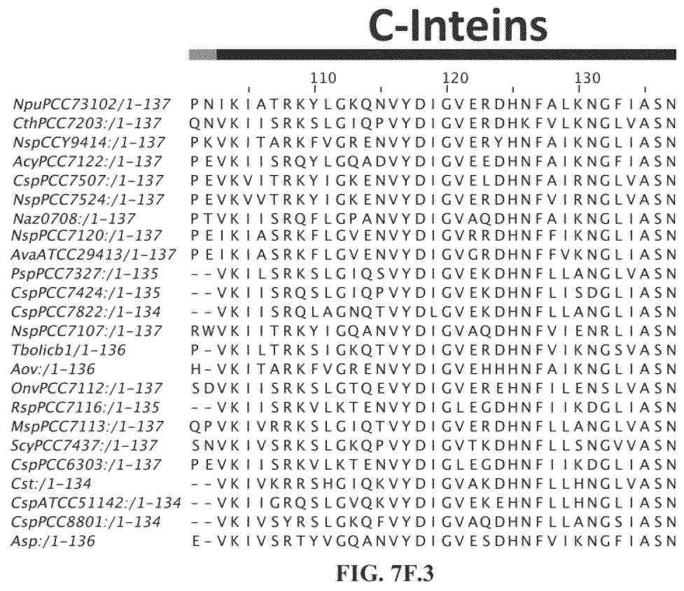

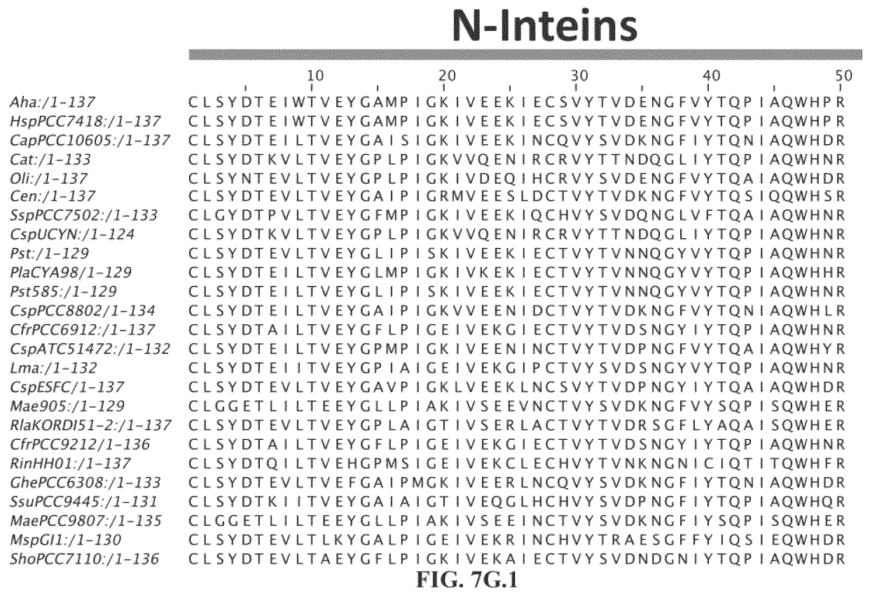

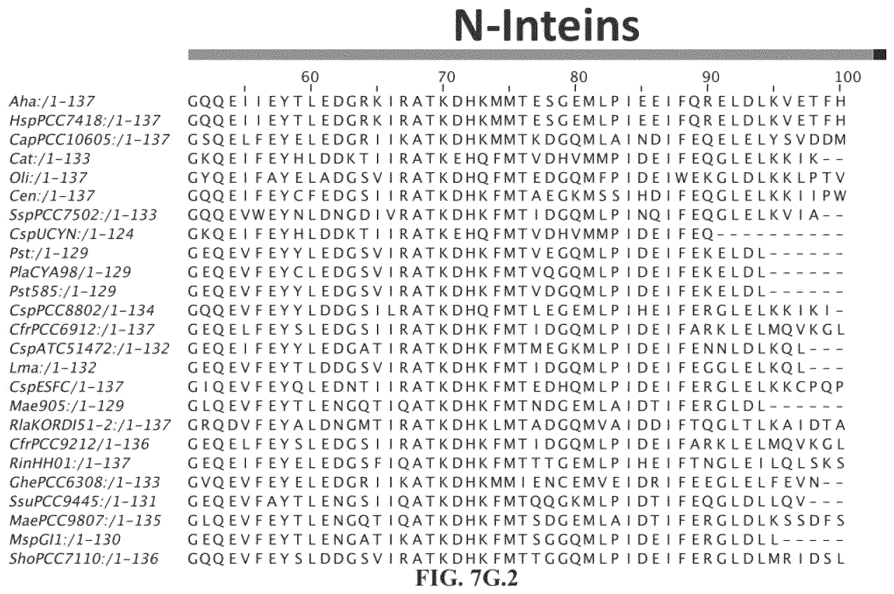

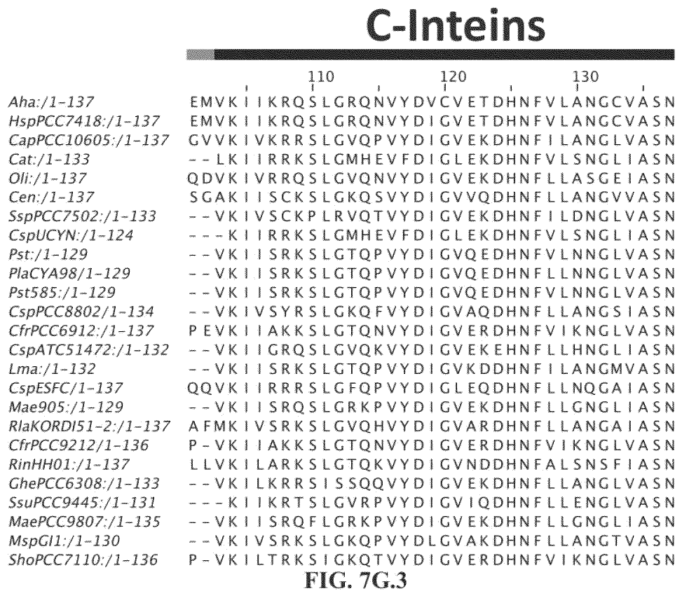

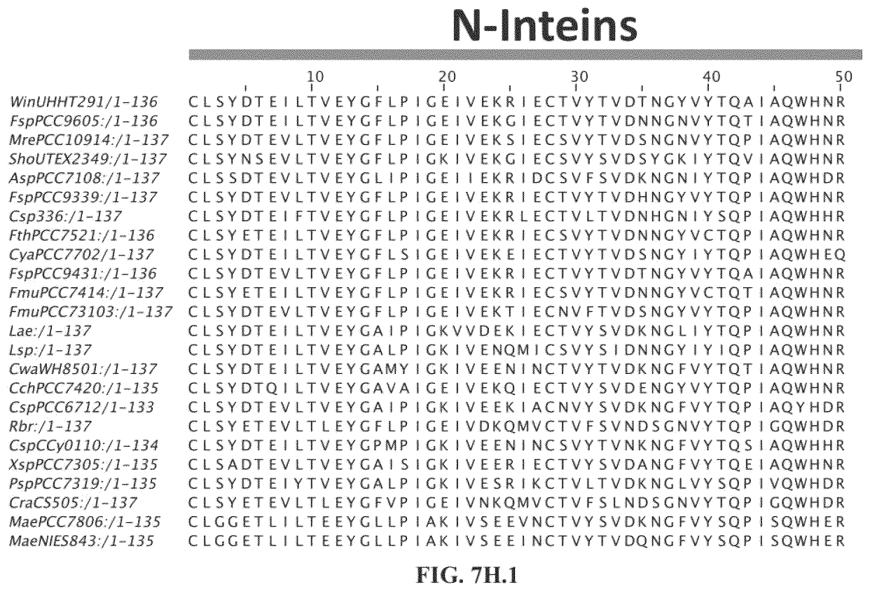

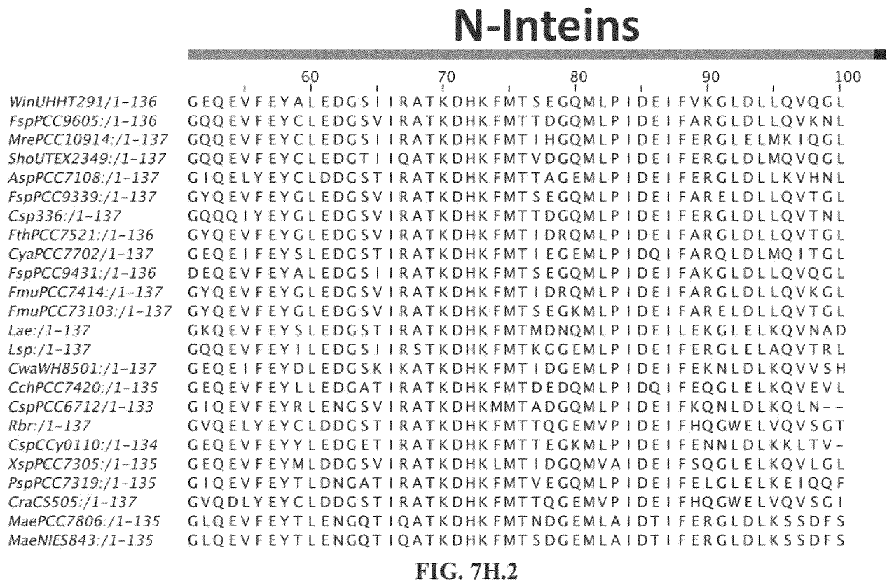

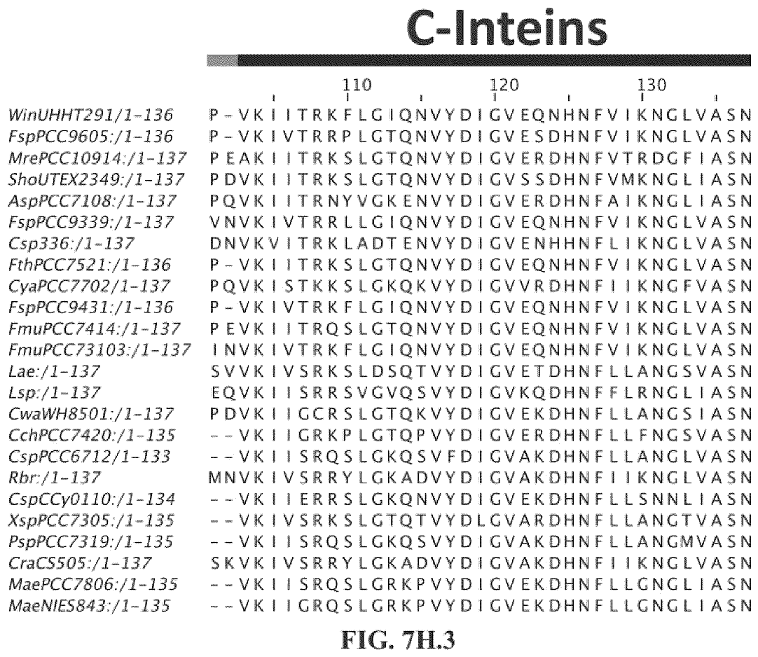

The ‘accelerator’ residues found to affect the splicing rate allow for an activity-guided approach to engineer a consensus DnaE intein. Consensus protein engineering is a tool applied to a homologous set of proteins in order to create a thermostable variant derived from the parent family.^{13,14} A multiple sequence alignment (MSA) is first generated from homologues of a particular protein, from which the most statistically frequent residue at each position is chosen as the representative in the consensus sequence. For the DnaE inteins, 105 sequences were identified through a BLAST¹⁵ search of the JGI¹⁶ and NCBI¹⁷ databases (FIGS. 7A.1 to 7A.3, 7B.1 to 7B.3, 7C.1 to 7C.3, 7D.1 to 7D.3, and 7E.1 to 7E.3). Next, the alignment was filtered to only contain sequences bearing the second shell indicators of fast splicing: K70, M75, M81, and S136. The 73 theoretically fast inteins left in the MSA (FIGS. 7F.1 to 7F.3, 7G.1 to 7G.3, and 7H.1 to 7H.3) were then used to generate a consensus fast DnaE intein sequence (Cfa) (FIG. 1). The various sequences disclosed in FIGS. 7A.1 to 7A.3, 7B.1 to 7B.3, 7C.1 to 7C.3, 7D.1 to 7D.3, 7E.1 to 7E.3, 7F.1 to 7F.3, 7G.1 to 7G.3, and 7H.1 to 7H.3 are presented below:

>NpuPCC73102/1-137
(SEQ ID NO: 5)
CLSYETEILTVEYGLLPIGKIVEKRIECTVYSVDNNGNIYTQP
VAQWHDRGEQEVFEYCLEDGSLIRATKDHKFMTVDGQMLPIDE
IFERELDLMRVDNLPN
(SEQ ID NO: 6)
IKIATRKYLGKQNVYDIGVERDHNFALKNGFIASN
>CthPCC7203:/1-137 Chroococcidiopsis
thermalis PCC 7203
(SEQ ID NO: 7)
CLSYDTEILTVEYGAIPIGKIVEERIECTVYSVDNNGFIYTQP
IAQWHNRGQQEVFEYCLEDGSURATKDHKFMTFEGKMLPIDEI
FEQELDLKQVKSIQN
(SEQ ID NO: 8)
VKIISRKSLGIQPVYDIGVERDHKFVLKNGLVASN
>NspCCY9414:/1-137 Nodularia spumigena
CCY9414 genome
(SEQ ID NO: 9)
CLSYDTEILTVEYGYIPIGEIVEKAIECSVYSVDNNGNVYTQP.
IAQWIINRGEQEVFEYSLEDGSTIRATKDHKFMTTDGQMLPID
EIFAQELDLLQVHGLPK
(SEQ ID NO: 10)
VKITARKFVGRENVYDIGVERYHNFAIKNGLIASN
>AcyPCC7122:/1-137 Anabaena cylindrica PCC
7122
(SEQ ID NO: 11)
CLSYDTEVLTVEYGFIPIGEIVEKRIECSIFSVDKNGNVYTQPI
AQWHNRGRQEIYEYCLDDGSKIRATKDHKFMTTAGEMLPIDEIF
ERDLDLLKVEGLPE
(SEQ ID NO: 12)
VKIISRQYLGQADVYDIGVEEDHNFAIKNGFIASN
>CspPCC7507:/1-137 Calothrix sp. PCC 7507,
complete genome
(SEQ ID NO: 13)
CLSYDTEVLTVEYGLLPIGEIVEKGIECRVFSVDNHGNVYTQP
IAQWHNRGQQEVFEYGLDDGSVIRATKDHKFMTTDGKMLPIDE
IFERGLDLLQVQGLPE
(SEQ ID NO: 14)
VKVITRKYIGKENVYDIGVELDHNFAIRNGLVASN
>NspPCC7524:/1-137 Nostoc sp. PCC 7524
(SEQ ID NO: 15)
CLSYDTEILTVEYGFLPIGEIVEKGIECTVFSVASNGIVYTQP
IAQWHNRGQQEIFEYCLEDGSIIRATKDHKFMTQDGQMLPIDE
IFACELDLLQVQGLPE
(SEQ ID NO: 16)
VKVVTRRYIGKENVYDIGVERDHNFVIRNGLVASN
>Naz0708:/1-137 ‘Nostoc azollae’ 0708
(SEQ ID NO: 17)
CLSYKTEVLIVEYGLIPIGEIVEKRIECSLFSVDENGNIYTQP
IAQWHHRGVQEVYEYCLDDGTIIRATKDHKFMTTIGEMLPIDE
IFERDLNLLQVNGLPT
(SEQ ID NO: 18)
VKIISRQFLGPANVYDIGVAQDHNFAIKNGLIASN
>NspPCC7120:/1-137 Nostoc sp. PCC 7120 DNA
(SEQ ID NO: 19)
CLSYDTEVLTVEYGFVPIGEIVEKGIECSVFSINNNGIVYTQP
IAQWHHRGKQEVFEYCLEDGSIIKATKDHKFMTQDGKMLPIDE
IFEQELDLLQVKGLPE
(SEQ ID NO: 20)
IKIASRKFLGVENVYDIGVRRDHNFFIKNGLIASN
>AvaATCC29413/1-137 Anabaena variabilis ATCC
29413
(SEQ ID NO: 21)
CLSYDTEVLTVEYGFVPIGEIVDKGIECSVFSIDSNGIVYTQP
IAQWHHRGKQEVFEYCLEDGSIIKATKDHKFMTQDGKMLPIDE
IFEQELDLLQVKGLPE
(SEQ ID NO: 22)
IKIASRKFLGVENVYDIGVGRDHNFFVKNGLIASN
>PspPCC7327:/1-135 Pleurocapsa sp. PCC 7327.
(SEQ ID NO: 23)
CLSYDTKILTVEYGAMPIGKIVEEQIDCTVYTVNQNGFVYTQP
IAQWHDRGKQEIFEYCLEDGSHRATKDHRFMTTDGQMLPIDKI
FEKGLDLKTINCD
(SEQ ID NO: 24)
VKILSRKSLG1QSVYD1GVEKDHNFLLANGLVASN
>CspPCC7424:/1-135 Cyanothece sp. PCC 7424
(SEQ ID NO: 25)
CLSYETQIMTVEYGLMPIGKIVEEQIDCTVYTVNKNGFVYTQP
IAQWHYRGEQEVFEYCLEDGSTRATKDHKFMTTDGQMLPIDEI
FEQGLELKQIHLS
(SEQ ID NO: 26)
VKIISRQSLGIQPVYDIGVEKDHNFLISDGLIASN
>CspPCC7822:/1-134 Cyanothece sp. PCC 7822
(SEQ ID NO: 27)
CLSYDTEILTVEYGPMPIGKIVEEQIECTVYTVDKNGLVYTQP
IAQWHHRGQQEVFEYCLEDGSIIRATKDIIKFMTDDGQMLP
IEEIFEKGLELKQIIL
(SEQ ID NO: 28)
VKIISRQLAGNQTVYDLGVEKDHNFLLANGLIASN
>NspPCC7107:/1-137 Nostoc sp. PCC 7107
(SEQ ID NO: 29)
CLSYDTQVLTVEYGLVPIGEIVEKQLECSVFTIDGHGYVYTQA
IAQWHNRGQQEVFEYGLEDGSVIRATKDIIKFMTTDGQMLPID
EIFERELDLLQVQGLRW
(SEQ ID NO: 30)
VKIITRKYIGQANVYDIGVAQDHNFVIENRLIASN
>TboIicb1/1-136 Tolypothrix bouteillei
Iicb1
(SEQ ID NO: 31)
CLSYDTEILTVEYGFLPIGKIVEKGIECNVYSVDKNGNIYTQP
IAQWHDRGEQEVFEYCLENGSVIRATKDHKFMTTSGEMLPIDE
IFERGLDLIRVEDLP
(SEQ ID NO: 32)
VKILTRKSIGKQTVYDIGVERDHNFVIKNGSVASN
>Aov:/1-136 Aphanizomenon ovalisporum
DnaE precursor (dnaE) gene
(SEQ ID NO: 33)
CLSADTEILTVEYGFLPIGEIVGKAIECRVYSVDGNGNGYTQS
IAQWHNRGEQEVFEYTLEDGSIIRATKDHKFMTTDGEMLPIDE
XFARQLDLMQVQGLH
(SEQ ID NO: 34)
VKITARKFVGRENVYDIGVEHHHNFAIKNGLIASN
>OnvPCC7112:/1-137 Oscillatoria nigro-viridis
PCC 7112
(SEQ ID NO: 35)
CLSYDTKILTVEYGPMAIGKIVEEKIECTVYSVDSNGYIYTQS
IAQWHRRGQQEVFEYCLEDGSIIRATKDHKFMTVGGQMLPIDE
IFEQGLDLKQINSSSD
(SEQ ID NO: 36)
VKHSRKSLGTQEVYDIGVEREHNFILENSLVASN
>RspPCC7116:/1-135 Rivularia sp. PCC 7116,
complete genome
(SEQ ID NO: 37)
CLSYDTEVLTEEFGLIPIGKIVEEKIDCTVYSVDVNGNVYSQP
IAQWHNRGMQEVFEYELEDGSTIRATKDHKFMTVDGEMLAIDE
IFEKGLELKRVGIY
(SEQ ID NO: 38)
VKIISRKVLKTENVYDIGLEGDHNFHKDGLIASN
>TerIMS101:/1-137 Trichodesmium erythraeum IMS101
(SEQ ID NO: 39)
CLTYETEIMTVEYGPLPIGKIVEYRIECTVYTVDKNGYIYTQP
IAQWHNRGMQEVYEYSLEDGTVIRATPEHKFMTEDGQMLPIDE
IFERNLDLKCLGTLEL
(SEQ ID NO: 40)
VKIVSRKLAKTENVYDIIVTKDHNFVLANGLIASN
>MspPCC7113.71-137 Microcoleus sp. PCC 7113,
(SEQ ID NO: 41)
CLSYDSEILTVEYGLMPIGKIVEEGIECTVYSVDSHGYLYTQP
IAQWHHRGQQEVFEYDLEDGSVIRATKDHKFMTSEGQMLAIDEI
FERGLELKQVKRSQP
(SEQ ID NO: 42)
VKIVRRKSEGIQTVYDIGVERDHNFLLANGLVASN
>ScyPCC7437:/1-137 Stanieria cyanosphaera PCC
7437
(SEQ ID NO: 43)
CLSYDTEILTVEYGAMPIGKIVKEQIECNVYTVNQNGFIYP
QAIAQWHERGKQEIFEYTLDNGLVIRATKDHKFMTIDGQML
PIDEIFERGLELQRINDYSN
(SEQ ID NO: 44)
VKIVSRKSLGKQPVYDIGVTKDHNFLLSNGVVASN
>CspPCC6303:/1-137 Calothrix sp. PCC 6303
(SEQ ID NO: 45)
CLSYDTEILTWEYGFLKIGEIVEKQILCSVFSVDEQGNVYT
QPIAQWHNRGLQELFAYQLEDGGVIRATKDHKFMTTDGQML
AIDEIFERQLDLFQVKGLPE
(SEQ ID NO: 46)
VKIISRKVLKTENVYDIGLEGDHNFIIKDGLIASN
>Cst/1-134 PCC7202: Cyanobacterium stameri
PCC 7202
(SEQ ID NO: 47)
CLSYDTEVLTVEYGVLPIGKIVEEQIQCTVYSVDQYGFVYT
QAIAQWHDRGEQEVFEYELENGATIKATKDHKMMTSDGQML
PIDQIFEQGLDLFMVSF
(SEQ 3D NO: 48)
VKIVKRRSHGIQKVYDIGVAKDHNFLLHNGLVASN
>CspATCC51142:/1-134 Cyanothece sp. ATCC 51142
(SEQ ID NO: 49)
CLSYDTEILTVEYGPMPIGKIVEENINCTYTTVDPNGFVYT
QAIAQWHYRGEQEIFEYYLEDGATIRATKDHKFMTMEGKML
PIDEIFENNLDLKQLTL
(SEQ ID NO: 50)
VKIIGRQSLGVQKVYDIGVEKEHNFLLHNGLIASN
>CspPCC8801:/1-134 Cyanothece sp. PCC 8801
(SEQ ID NO: 51)
CLSYDTEILTVEYGAIPIGKVVEENIDCTVYTVDKNGFVYT
QNIAQWHLRGQQEVFEYYLDDGSTLRATKDHQFMTLEGEML
PIHEIFERGLELKKIKI
(SEQ ID NO: 52)
VKIVSYRSLGKQFVYDIGVAQDHNFLLANGSIASN
>Asp:/1-136 Anabaena sp. 90 chromosome
(SEQ ID NO: 53)
CLSYDTEILTVEYGFLEIGEIVEKQIECKVYTIDSNGMLYT
QSIAQWHNRGQQEVYEYLLENGAIIRATKDHKFMTEAGQML
PIDEIFAQGLDLLQVGVAE
(SEQ ID NO: 54)
VKIVSRTYVGQANVYDIGVESDHNFVIKNGFIASN
>Aha:/1-137 Aphanothece halophytica
(SEQ ID NO: 55)
CLSYDTEIWTVEYGAMPIGKIVEEKIECSVYTVDENGFVYT
QPIAQWHPRGQQEIIEYTLEDGRKIRATKDHKMMTESGEML
PIEEIFQRELDLKVETFHEM
(SEQ ID NO: 56)
VKIIKRQSLGRQNVYDVCVHDIANFVLANGCVASN
>HspPCC7418:/1-137 Halothece sp. PCC 7418
(SEQ ID NO: 57)
CLSYDTEIWTVEYGAMPIGKIVEEKTECSVYTVDENGFVYTQP
IAQWHPRGQQEIIEYTLEDGRKIRATKDHKMMTESGEMLPIEE
IFQRELDLKVETFHEM
(SEQ ID NO: 58)
VKHKRQSLGRQNVYDIGVETDIINFVLANGCVASN
>CapPCC 1060571-137 Cyanobacterium aponinum
PCC 10605
(SEQ ID NO: 59)
CLSYDTEILIYEYGAISIGKIVEEKINCQVYSVDKNGFIYTQN
IAQWHDRGSQELFEYELEDGRIIKATKDHKMMTKDGQMLAIND
IFEQELELYSVDDMGV
(SEQ ID NO: 60)
VKIVKRRSLGVQPVYDIGVEKDHNFILANGLVASN
>Cat:/1-133 Candidate Atelocyanobacterium
thalassa isolate
(SEQ ID NO: 61)
CLSYDTKVLTVEYGPLPIGKVVQENIRCRVYTTNDQGLIYTQ
PIAQWHNRGKQEIFEYHLDDKTIIRATKEHQFMTVDHVMMPI
DEIFEQGEELKKIK
(SEQ ID NO: 62)
LKIIRRKSLGMHEVFDIGLEKDHNFVLSNGLIASN
>01171-137 Oscillaloria limnetica ‘Solar Lake’
DnaE precursor
(SEQ ID NO: 63)
CLSYNTEVLTVEYGPLPIGKIVDEQIHCRVYSVDENGFVYTQ
AIAQWHDRGYQEIFAYELADGSVIRATKDHQFMTEDGQMFPI
DEIWEKGLDLKKLPTVQD
(SEQ ID NO: 64)
VKIVRRQSLGVQNVYDIGVEKDHNFLLASGEIASN
>Cen:/1-137 Cyanobacterium endosymbiont of
Epithemia turgida
(SEQ ID NO: 65)
CLSYDTEVLTVEYGAIPIGRMVEESLDCTVYTVDKNGFVYTQ
SIQQWHSRGQQEIFEYCFEDGSHRATKDHKFMTAEGKMSSIH
DIFEQGLELKKIIPWSG
(SEQ ID NO: 66)
AKIISCKSLGKQSVYDIGVVQDHNFLLANGVVASN
>SspPCC7502:/1-133 Synechococcus sp. PCC 7502
(SEQ ID NO: 67)
CLGYDTPVLTVEYGFMPIGKIVEEKIQCHVYSVDQNGLVFTQ
AIAQWHNRGQQEVWEYNLDNGDIVRATKDHKFMTIDGQMLPI
NQIFEQGLELKVIA
(SEQ ID NO: 68)
VKIVSCKPLRVQTVYDIGVEKDHNFILDNGLVASN
>DsaPCC8305:/1-134 Dactylococcopsis salina
PCC 8305
{SEQ ID NO: 69)
CLSYDTEVLTEEYGAIPIGKIVEERMNCHVYSVDENGFIYSQP
IAQWHPRGEQEWEYTLEDGKIIRATADHKMMTETGEMLPIEQI
FQQQLDLKISNQ
(SEQ ID NO: 70)
VKIINRQSLGKQTVYDIGVEKDHNFILGNGLVASN
>CstPCC7417:/1-337 Cylindrospermum stagnale
PCC 7417
(SEQ ID NO: 71)
CLSYDTEILTVEYGFIPIGEIVEKRIECSVYSVDNHGNVYTQP
IAQWHNRGLQEVFEYCLEDGSTIRATKDHKFMTTDKEMLPIDE
IFERGLDLLRVEGLPI
(SEQ ID NO: 72)
VKHMRSYVGRENYYDIGVERDHNFVAKNGLIAAN
>SspPCC6803:/1-137 Synechocystis sp. PCC 6803
(SEQ ID NO: 73)
CLSFGTEILTVEYGPLPIGKIVSEEINCSVYSVDPEGRVYTQ
AIAQWHDRCEQEVLEYELEDGSVIRATSDHRFLTTDYQLLAI
EEIFARQLDLLTLENIKQ
(SEQ ID NO: 74)
VKYTGRRSLGVQRIFDIGLPQDHNFLLANGAIAAN
>GspPCC7401:/1-137 Geitlerinema sp. PCC 7407
(SEQ ID NO: 75)
CLSYETPVMTVEYGPLPIGRIVEEQLDCTVYSVDEQGHVYTQ
PVAQWHHRGLQEWEYELEDGRRLRATADHRFMTETGEMLPLA
EIFERGLELRQVALRVP
(SEQ ID NO 76)
VKIVSRRSLGMQLVYDIGVAADHNFVLADGLIAAN
>SspPCC6714:/1-137 Synechocystis sp. PCC 6714
(SEQ ID NO: 77)
CLSFDAEILTVEYGPLSIGKIVGEEINCSVYSVDPQGRIYTQ
AIAQWHDRGVQEVFEYELEDGSVIRATPDHRFLTTDYELLAI
EEIFARQMDLLTLTNLKL
(SEQ ID NO: 78)
VKVVRRRSLGMHRVFDIGLAQDHNFLLANGAIAAN
>MaePCC7806:/1-135 Microcystis aeruginosa
PCC 7806
(SEQ ID NO: 79)
CLGGETLILTEEYGLLPIAKIVSEEVNCTVYSVDKNGFVYSQ
PISQWHERGLQEVFEYTLENGQTIQATKDHKFMTNDGEMLAI
DTIFERGLDLKSSDFS
(SEQ ID NO: 80)
VKIISRQSLGRKPVYDIGVEKDIINFLLGNGLIASN
>MaeNIES843:/1-135 Microcystis aemginosa
NIES-843 DNA
(SEQ ID NO: 81)
CLGGETLILTEEYGLLPIAKIVSEEINCTVYTVDQNGFVYSQI
MSQWHERGLQHVFEYTLENGQTIQATKDHKFMTSDGEMLAIDT
IFERGLDLKSSDFS
(SEQ ID NO: 82)
VKTTGRQSLGRKPVYDIGVEKDHNFLLGNGLIASN
>AmaMBIC11017:/1-137 Acaryochloris marina
MBIC11017.
(SEQ ID NO: 83)
CLSYDIPVLTLEYGWLPIGQVVQEQIECQVFSINERGHLYTQP
IAQWHHRGQQEVFEYTLADGSTIQATAEHQFMTTDGQMYPVQQ
IFEEGLSLKQLPLPWQ
(SEQ ID NO: 84)
VKIIQRRSLGLQSVYDIGLAQDHNFVMANGWVAAN
>LspPCC7376:/1-137 Leptolyngbya sp. PCC 7376
(SEQ ID NO: 85)
CLDGETPIVTVEYGVLPIREIVEKELLCSVYSIDENGFVYTQP
VEQWHQRGDRQMFEYQLDNGGVIRATPDHKFLTTEGEMVAIDE
IFEKGLNLAEFAPADL
(SEQ ID NO: 86)
VKILRRHSIGKAKTYDIGVSKNHNFLLANGLFASN
>SelPCC6301:/1-137 Synechococcus elongatus
PCC 6303
(SEQ ID NO: 87)
CLAADTEVLTVEYGPIAIGKLVEENIRCQVYCCNPDGYIYSQ
PICQWHQRGEQEVIEYELSDGRHRATADHRFMTEEGEMLSLD
EIFERSLELKQIPTPLL
(SEQ ID NO: 88)
VKFVRRRSLGVQPVYDLGVATVHNFVLANGLVASN
>SspPCC6312:/1-137 Synechococcus sp. PCC 6312
(SEQ ID NO: 89)
CLSADTELYTVEYGWLPICRLVEEQIECQVLSVNAHGHVYSQ
PIAQWHRRAWQEVFEYQLETGGTIKATTDHQFLTTDGQMYRI
EDIFQRGLDLWQLPPDRF
(SEQ ID NO: 90)
VKIISRCSLGIQPVYDIGVAQDHNFVIRGGLVASN
>Tel:/1-137 Thermosynechococcus elongatus
BP-1 DNA
(SEQ ID NO: 91)
CLSGETAVMTVEYGAVPIRRLVQERLSCHVYSLDGQGHLYTQ
PIAQWHFQGFRPVYEYQLEDGSTICATPDHRFMTTRGQMLPI
EQIFQEGLELWQVAIAPR
(SEQ ID NO:92)
GKIVGRRLMGWQAVYDIGLAADHNFVLANGAIAAN
>Tsp:/1-137 Thermosynechococcus sp. NK55 genome
(SEQ ID NO: 93)
CLSGETAVMTVEYGAVPIRRLVQERLTCHVYSLDAQGHLYT
QPIAQWHFQGFRPVYEYQLEDGSTIWATPDHRFMTTRGQML
PIEQIFQEGLELWQGPIAPS
(SEQ ID NO: 94)
CKIVGRQLVGWQAVYDIGVARDHNFLLANGAIAAN
>Tvu:/1-137 Thermosynechococcus vulcanus
DnaE precursor
(SEQ ID NO: 95)
CLSGETAVMTVEYGAIPIRRLVQERLICQYYSLDPQ
GHLYTQPIAQWHFQGFRPVYAYQLEDGSTICATPDH
RFMTTSGQMLPIEQIFREGLELWQVAIAPP
(SEQ ID NO: 96)
CKIVGRRLVGWQAVYDIGLAGDHNFLLANGAIAAN
>SspPCC7002:/1-137 Synechococcus sp. PCC 7002
(SEQ ID NO: 97)
CLAGGTPVVTVEYGVLPIQITVEQELLCHVYSVDA
QGLIYAQLIEQWHQRGDRLLYEYELENGQMIRATP
DIIRFLTTFGELLPIDEIFTQNLDLAAWAVPDS
(SEQ ID NO: 98)
VKIIRRKFIGHAPTYDIGLSQDHNFLLGQGLIAAN
>ShoPCC7110:/1-136 Scytonema hofmanni
PCC 7110 contig00136
(SEQ ID NO: 99)
CLSYDTEVLTAEYGFLPIGKIVEKAIECTVYSVDN
DGNIYTQPIAQWHDRGQQEVFEYSLDDGSVIRATK
DHKFMTTGGQMLPIDEIFERGLDLMRIDSLP
(SEQ ID NO: 100)
VKILTRKSIGKQTVYDIGVERDHNFVIKNGLVASN
>WinUHHT291/1-136 Westiella intricata
UHHT-29-1
(SEQ ID NO: 101)
CLSYDTEILTVEYGFLPIGEIVEKRIECTVYTVDT
NGYVYTQAIAQWHNRGEQEVFEYALEDGSURATKD
HKFMTSEGQMLPIDEIFVKGLDLLQVQGLP
(SEQ ID NO: 102)
VKIITRKFLGIQNVYDIGVEQNHNFVIKNGLVASN
>FspPCC9605:/1-136 Fischerella sp.
PCC 9605 FIS9605DRAFT
(SEQ ID NO: 103)
CLSYDTEILTVEYGFLPIGEIVEKGIECTVYTVDN
NGNVYTQTIAQWHNRGQQEVFEYCLEDGSVIRATK
DHKFMTTDGQMLPIDEIFARGLDLLQVKNLP
(SEQ ID NO: 104)
VKIVTRRPLGTQNVYDIGVESDHNFVIKNGLVASN
>MrePCC10914:/1-137 Mastigocladopsis repens
PCC 10914
(SEQ ID NO: 105)
CLSYDTEVLTVEYGFLPIGEIVEKSIECSVYTVDS
NGNVYTQPIAQWHNRGQQEVFEYCLEDGSIIRATK
DHKFMTIHGQMLPIDEIFERGLELMKIQGLPE
(SEQ ID NO:106)
AKIITRKSLGTQNVYDlGVERDHNFVTRDGFIASN
>ShoUTEX2349:/1-137 [Scytonema hofmanni]
UTEX 2349
(SEQ ID NO: 107)
CLSYNSEVETVEYGFLPIGKIVEKGIECSVYSVDS
YGKIYTQVIAQWHNRGQQEVFEYCLEDGTHQATKD
HKFMTVDGQMLPIDEIFERGLDLMQVQGLPD
(SEQ ID NO: 108)
VKNTRKSLGTQNVYDIGVSSDIINFVMKNGLIASN
>AspPCC7108:/1-137 Anabaena sp. PCC 7108
Ana7108scaffold_2_Cont3
(SEQ ID NO: 109)
CLSSDTEVLTVEYGLIPIGEIIEKRIDCSVFSVDK
NGNIYTQPIAQWHDRGIQELYEYCLDDGSTIRATK
DHKFMTTAGEMLPIDEIFERGLDLLKVHNLPQ
(SEQ ID NO: 110)
VKHTRNYVGKENVYDIGVERDHNFAIKNGLIASN
>FspPCC9339:/1-137 Fischerella sp.
PCC 9339 PCC9339DRAFT
(SEQ ID NO: 111)
CLSYDTEVLTVEYGFLPIGEIVEKRIECTVYTVDH
NGYVYTQPIAQWHNRGYQEVFEYGLEDGSVIRATK
DHKFMTSEGQMLPIDEIFARELDLLQVTGLVN
(SEQ ID NO: 112)
VKIVTRRLLGIQNVYDIGVEQNHNFVIKNGLVASN
>Csp336:/1-137 Calothrix sp. 336/3
(SEQ ID NO: 113)
CLSYDTEIFTVEYGFLPIGEIVEKRLECTVLTVDN
HGNTYSQPIAQWHHRGQQQIYEYGLEDCSVIRATK
DHKFMTTDGQMEPIDEIFERGLDLLQVTNLDN
(SEQ ID NO: 114)
VKVITRKLADTENVYDIGVENHHNFLIKNGLVASN
>FthPCC7521:/1-136 Fischerella thennalis
PCC 7521
(SEQ ID NO: 115)
CLSYETEILTVEYGFLPIGEIVEKRIECSVYTVDN
NGYVCTQPIAQWHNRGYQEVFEYGLEDGSVIRATK
DHKFMTIDRQMLPIDEIFARGLDLLQVTGLP
(SEQ ID NO: 116)
VKIITRKSLGTQNVYDIGVEQNHNFVIKNGLVASN
>CyaPCC7702/1-137 cyanobacterium PCC 7702
Chl7702
(SEQ ID NO: 117)
CLSYDTEILTVEYGFLSIGEIVEKEIECTVYTVDS
NGYIYTQPIAQWHEQGEQEIFEYSLEDGSTIRATK
DHKFMTIEGEMLPIDQIFARQLDLMQITGLPQ
(SEQ ID NO: 118)
VKISTKKSLGKQKVYDIGVVRDHNFIIKNGFVASN
>FspPCC9431:/1-136 Fischerella sp. PCC 9431
(SEQ ID NO: 119)
CLSYDTEVLTVEYGFLPIGEIVEKRIECTVYTVDT
NGYVYTQAIAQWHNRDEQEVFEYALEDGSIIRATK
DHKFMTSEGQMLPIDEIFAKGLDLLQVQGLP
(SEQ ID NO: 120)
VKIVTRKFLGIQNYYDIGVEQNHNFVIKNGLVASN
>FmuPCC7414:/1-37 Fischerella muscicola
PCC 7414
(SEQ ID NO: 121)
CLSYETEILTVEYGFLPIGEIVEKRIECSVYTVDN
NGYVCTQTIAQWHNRGYQEVFEYGLEDGSVIRATK
DHKFMTIDRQMLPIDEIFARGLDLLQVKGLPE
(SEQ ID NO: 122)
VKIITRQSLGTQNVYDIGVEQNHNFVIKNGLVASN
>FmuPCC73103:/1-137 Fischerella muscicola
SAG 1427-1 = PCC 73103
(SEQ ID NO: 123)
CLSYDTEVLTVEYGFLPIGEIVEKTIECNVFTVDS
NGYVYTQPIAQWHNRGYQEVFEYGLEDGSVIRATK
DHKFMTSEGKMIPIDETFARELDLLQVTGLIN
(SEQ ID NO: 124)
VKIVFRKFLGIQNVYDIGVEQNHNFVIKNGLVASN
>Lae:/1-l37 Lyngbya aestuarii BL J
laest3.contig.3
(SEQ ID NO: 125)
CLSYDTEILTVEYGAIPIGKVVDEKIECTVYSVDK
NGLIYTQPIAQVVHNRGKQEVFEYSLEDGSTIRAT
KDHKFMTMDNQMLPIDEILEKGLELKQVNADSV
(SEQ ID NO: 126)
VKIVSRKSLDSQTVYDIGVETDFINFLLANGSVASN
>MspPCC7126:/1-135 Microchaete sp.
PCC 7126
(SEQ ID NO: 127)
CLSYKTQVLTVEYGLLAIGEIVEKNIECSVFSVDI
HGNVYTQPIAQWHHRGQQEVFEYGLEDGSIIRATK
DHKFMTTQGEMLPIDEIFARGLDLLQVKGV
(SEQ ID NO: 128)
VKIITRKYIGKENVYDIGVEQDHNFAIKNGLIAAN
>Lsp:/1-137 Leptolyngbya sp. JSC-1
(SEQ ID NO: 129)
CLSYDTEILTVEYGALPIGKIVENQMICSVYSIDN
NGYIYIQPIAQWHNRGQQEVFEYILEDGSIIRSTK
DHKFMTKGGEMEPIDEIFERGLELAQVTRLEQ
(SEQ ID NO: 130)
VKIISRRSVGVQSVYDIGVKQDHNFFLRNGLIASN
>CwaWH8501:/1-137 Crocosphaera watsonii
WH8501
(SEQ ID NO: 131)
CLSYDTEILTVEYGAMYIGKIVEENINCTVYTVDK
NGFVYTQTIAQWHNRGEQEIFEYDLEDGSKIKAT
KDHKFMTIDGEMLPIDEIFEKNLDLKQVVSHPD
(SEQ ID NO: 132)
VKIIGCRSLGTQKVYDIGVEKDHNFLLANGSIASN
>CchPCC7420:/1-135 Coleofasciculus chthonoplastes
PCC 7420 (Mcht)
(SEQ ID NO: 133)
CLSYDTQILTVEYGAVAIGEIVEKQIECTVYSVDE
NGYVYTQPIAQWHNRGEQEVFEYLLEDGATIRATK
DHKFMTDEDQMLPIDQIFEQGLELKQVEVL
(SEQ ID NO: 134)
VKIIGRKPLGTQPVYDIGVERDHNFLLFNGSVASN
>CspPCC6712/1-133
(SEQ ID NO: 135)
CLSYDTEVLTVEYGAIPIGKIVEEKIACNVYSVDK
NGFVYTQPIAQYHDRGIQEVFEYRLENGSVIRATK
DHKMMTADGQMLPIDEIFKQNLDLKQLN
(SEQ ID NO: 136)
VKIISRQSLGKQSVFDIGVAKDFINFLLANGLVASN
>AflNIES81:/1-132 Aphanizomenon flos-aquae
NIES-81
(SEQ ID NO: 137)
CLSYDTEILTVEYGFLQIGEIVEKQIECKVYTVDS
NGILYTQSIAQWHNRGQQEVYEYLLENGAIIRATK
DHKFMTEEGQMLPIDEIFSQGLDLLQV
(SEQ ID NO: 138)
VKIISRTYVGQANVYDIGVENDHNFVIKNGFIAAN
>Rbr:/1-137 Raphidiopsis brookii D9 D9_5,
(SEQ ID NO: 139)
CLSYETEVLTLEYGFLPIGEIVDKQMVCTVFSVND
SGNVYTQPIGQWHDRGVQELYEYCLDDGSTIRATK
DHKFMTIQGEMVPIDEIFHQGWELVQVSGTMN
(SEQ ID NO: 140)
VKIVSRRYLGKADVYDIGVAKDHNFHKNGLVASN
>CspCCy0110:/1-134 Cyanothece sp.
CCY0110 1101676644604
(SEQ ID NO: 141)
CLSYDTEILTVEYGPMPIGKIVEENINCSVYTVNK
NGFVYTQSIAQWHHRGEQEVFEYYLEDGETIRATK
DHKFMTTEGKMLPIDEIFENNLDLKKLTV
(SEQ ID NO: 142)
VKIIERRSLGKQNVYDIGVEKDHNFLLSNNLIASN
>XspPCC7305:/1-135 Xenococcus sp.
PCC 7305
(SEQ ID NO: 143)
CLSADTEVLTVEYGAISIGKIVEERIECTVYSVDA
NGFVYTQEIAQWHNRGEQEVFEYMLDDGSVIRATK
DHKLMTIDGQMVAIDEIFSQGLELKQVLGL
(SEQ ID NO: 144)
VKIVSRKSLGTQTVYDLGVARDHNFLLANGTVASN
>PspPCC7319:/1-135 Pleurocapsa sp.
PCC 7319
(SEQ ID NO: 145)
CLSYDTEIYTVFYGALPIGKIVESRIKCTVLTVDK
NCLVYSQPIVQWHDRGIQEVFEYTLDNGATIRATK
DHKFMTVEGQMLPIDEIFELGLELKEIQQF
(SEQ ID NO: 146)
VKIISRQSLGKQSVYDIGVAKDHNFLLANGMVASN
>CraCS505:/1-137 Cylindrospermopsis
raciborskii CS-505
(SEQ ID NO: 147)
CESYETEVLTLEYGFVPIGEIVNKQMVCTVFSLN
DSGNVYTQPIGQWHDRGVQDLYEYCLDDGSTIRA
TKDHKFMTTQGEMVPIDEIFHQGWELVQVSGISK
(SEQ ID NO: 148)
VKIVSRRYLGKADVYDIGVAKDHNFIIKNGLVASN
>SmaPCC6313/1-129 Spirulina major
PCC 6313
(SEQ ID NO: 149)
CLTYDTLVLTVEYGPVPIGKLVEAQINCQVYSV
DANGFIYTQAIAQWHDRGQRQVYEYTLEDGSTI
RATPDHKFMTATGEMLPIDQIFEQGLDL
(SEQ ID NO: 150)
VKIIHRRALPPQSVYDIGVERDHNFLLPSGWVASN
>SsuPCC9445:/1-131 Spirulina subsalsa
PCC 9445
(SEQ ID NO: 151)
CLSYDTKIITVEYGAIAICTTVEQGLHCFWYSVD
PNGFIYTQPIAQWHQRGEQEVFAYTLENGSIIQA
TKDHKFMTQQGKMLPIDTIFEQGLDLLQ
(SEQ ID NO: 152)
VKIIKRTSLGVRPVYDIGVIQDHNFLLENGLVASN
>MaePCC9807:/1-135 Microcystis aeruginosa 9807
(SEQ ID NO: 153)
CLGGETLILTEEYGLLPIAKIVSEEINCTVYSVD
KNGFIYSQPTSQWHERGLQEVFEYTLENGQTIQA
TKDHKFMTSDGEMLAIDTIFERGLDLKSSDFS
(SEQ ID NO: 154)
VKUSRQFLGRKPVYDIGVEKDHNFLLGNGLIASN
>MspGI1:/1-130 Myxosarcina sp. GI1 contig_13
(SEQ ID NO: 155)
CLSYDTEVLTLKYGALPIGEIVEKRINCHVYTRA
ESGFFYIQSIEQWHDRGEQEVFEYTLENGATIKA
TKDHKFMTSGGQMLPIDEIFERGLDLL
(SEQ ID NO: 156)
VKIVSRKSLGKQPVYDLGVAKDHNFLEANGTVASN
>LspPCC6406:/1-136 Leptolyngbya sp. PCC 6406
(SEQ ID NO: 157)
CLSADTQLLTVEYGPLEIGRIVEEQIACHVYSVDA
NGFVYTQPIAQWHSRGEQEIFEYQLEDGRTLRATA
DHKFMTTTGEMGRINDIFEQGLDLKQIDLPQ
(SEQ ID NO: 158)
VKWSRQSLGVQPVYDIGVATDHNFLLADGLVASN
>AspCCMEE5410:/1-132 Acaryochloris sp.
CCMEE 5410
(SEQ ID NO: 159)
CLSYDTPVLTLEYGWLPIGQVVQEQIECQVFSIN
ERGHLYTQPIAQWHHRGQQEVFEYTLTDGSTIQA
TAEHQFMTTDGQMYPIQQIFEEGLSLKQL
(SEQ ID NO: 160)
VKITFQRRSLGLQSVYDIGLAQDHNFVIANGWVAAN
>GhePCC6308:/1-133 Geminocystis herdmanii
PCC 6308
(SEQ ID NO: 161)
CLSYDTEVETVEFGAIPMGKIVEERLNCQVYSVD
KNGFIYTQNIAQWHDRGVQEVFEYELEDGRIIKA
TKDHKMMIENCEMVEIDRIFEEGLELFEVN
(SEQ ID NO: 162)
VKILKRRSISSQQVYD1GVEKDHNFLLANGLVASN
>NnoPCC7104:/1-133 Nodosilinea nodulosa
PCC 7104
(SEQ ID NO: 163)
CLSADTELLTLEYGPLTIGEIVAKRIPCHVFSVDE
SGYVYTQPVAQWHQRGHQEVFEYQLDDGTTIRATA
DHQFMTELGEMMAIDEIFQRGLELKQVE
(SEQ ID NO: 164)
VKIISRQSLGVQPVYDIGVARDHNFLLADGQVASN
>RlaKORDI51-2:/1-137 Rubidibacter lacunae
KORDI 51-2
(SEQ ID NO 165)
CLSYDTEVLTVEYGPLAIGTIVSERLACTVYTVDR
SGFLYAQAISQWHERGRQDVFEYALDNGMTIRATK
DHKLMTADGQMVAIDDIFTQGLTLKAIDTAAF
(SEQ ID NO: 166)
MKIVSRKSLGVQHVYDIGVARDHNFLLANGAIASN
>CfrPCC9212/1-136 Chlorogloeopsis fritschii
PCC 9212
(SEQ ID NO: 167)
CLSYDTAILTVEYGPLPIGEIVEKGIECTVYTVDS
NGYIYTQPIAQWHNRGEQELFEYSLEDGSIIRATK
DHKFMTIDGQMLPIDEIFARKLELMQVKGLP
(SEQ ID NO: 168)
VKIIAKKSLGIQNVYDIGVERDHNFVIKNGLVASN
>RinHH01:/1-137 Richelia intracellularis
HH01 WGS project
(SEQ ID NO: 169)
CLSYDTQILTVEHGPMSIGEIVEKCLECHVYTVN
KNGNICIQTITQWHFRGEQEIFEYELEDGSFIQA
TKDHKFMTTTGEMLPIHEIFTNGLEILQLSKSLL
(SEQ ID NO: 170)
VKILARKSLGTQKVYDIGVNDDHNFALSNSFIASN
>SspPCC7117/1-137
(SEQ ID NO: 171)
CLAGDTPVVTVEYGVLPIQTIVEQELLCQVYSVDA
QGLIYTQPIEQWHNRGDRLLYEYELENGQMIRATP
DHKFLTITGELLPIDEIFTQNLDLAAWAVPDS
(SEQ ID NO: 172)
VKIIRRKFIGHAPTYDIGLSQDHNFLLGQGLIAAN
>SspPCC8807/1-137
(SEQ ID NO: 173)
CLAGDIPVVTVEYGVLPIQTIVEQELLCHVYSVDA
QGLIYTQPIEQWHQRGDRFLYEYELENGQMIRATP
DHKFLTTTGKLLPIDEIFTQNLDLAAWAVPDS
(SEQ ID NO: 174)
VKHRRKFIGHAPTYDIGLSQDHNFLLGQGFIAAN
>SspNKBG042902:/1-137 Synechococcus sp.
NKBG 042902
(SEQ ID NO: 175)
CLAGDTPVVTVEYGVLPIQTIVEQELLCHVYSVDA
QGLIYTQPIEQWHQRGDRLLYEYELENGQMIRATP
DHKFLTTTGELLPIDEIFTQNLDLAAWAVPDS
(SEQ ID NO: 176)
VKILRRKFIGRAPTYDIGLSQDHNFLLGQGLVAAN
>SspNKBG15041:/1-129 Synechococcus sp.
NKBG15041
(SEQ ID NO: 177)
CLAGDTPVVTVEYGVLPIRTIVDQELLCHVYSLDP
QGFIYAQPVEQWHRRGDRLLYEYELETGAVIRATP
DHKFLTATGEMLPIDEIFVRNLDL
(SEQ ID NO: 178)
VKIIRRNLIGEAATYDIGLGKDHNFLLGQGLIASN
>SspPCC73109/1-130
(SEQ ID NO: 179)
CLAGGTPVVTVEYGVLPIQTIVEQELLCHVYSVDA
QGLIYTQPIEQWHQRGDRLLYEYELENGQMIRATP
DHKFLTTTGELLPIDEIFTQNLDLL
(SEQ ID NO: 180)
VKIIRRKFIGHAPTYDIGLSQDHNFLLGQGLIAAN
>SspPCC7003/1-130
(SEQ ID NO: 181)
CLAGDTPVVTVEYGVLPIQTIVEQELLCHVYSVDA
QGLIYTQPIEQWHKRGDRLLYEYELENGQHRATPD
HKFLTTTGEMRPIDEIFAKNLSLL
(SEQ ID NO: 182)
VKIIRRKFVGHAPTYDIGLSQDHNFLLGQGLIAAN
>CspPCC8802/1-134: Cyanothece sp. PCC 8802
(SEQ ID NO: 183)
CLSYDTEILTVEYGAIPIGKVVEENIDCTVYTVDKN
GFVYTQNIAQWHLRGQQEVFEYYLDDGSILRATKDI
IQFMTLEGEMLPTHEIFERGLELKKTKI
(SEQ ID NO: 184)
VKIVSYRSLGKQFVYDIGVAQDHNFLLANGSIASN
>SelPCC7942:/1-137 Synechococcus elongatus
PCC 7942
(SEQ ID NO: 185)
CLAADTEVLTVEYGPIAIGKLVEENIRCQVYCCNPD
GYIYSQPIGQWHQRGEQEVIEYELSDGRIIRATADH
RFMTEEGEMLSLDEIFERSLELKQIPTPLL
(SEQ ID NO: 186)
VKIVRRRSLGVQPVYDLGVATVHNFVLANGLVASN
>CfrPCC6912:/1-137 Chlorogloeposis
Fritschii PCC 6912
(SEQ ID NO: 187)
CLSYDTAILTVEYGFLPIGEIVEKGIECTVYTVDSN
GYIYTQPIAQWHNRGEQELFEYSLEDGSIIRATKDH
KFMTIDGQMLPIDEIFARKLELMQVKGLPE
(SEQ ID NO: 188)
VKHAKKSLGTQNVYDIGVERDHNFVIKNGLVASN
>CspATC51472:/1-132 Cyanothece sp.
ATCC 51472
(SEQ ID NO: 189)
CLSYDTEILTVEYGPMPIGKIVEENINCTVYTVDPN
GFVYTQAIAQWHYRGEQEIFEYYLEDGATIRATKDH
KFMTMEGKMLPIDEIFENNLDLKQL
(SEQ ID NO: 190)
VKHGRQSLGVQKVYDIGVEKEHNFLLHNGLIASN
>Lma:/1-132 Lyngbya majuscula
(SEQ ID NO: 191)
CLSYDTEIITVEYGPIAIGEIVEKGIPCTVYSVDSN
GYVYTQPIAQWHNRGEQEVFEYTLDDGSVIRATKDH
KFMTIDGQMLPIDEIFEGGLELKQL
(SEQ ID NO: 192)
VKIISRKSLGTQPVYDIGVKDDHNFILANGMVASN
>CspESFC/1-137
(SEQ ID NO: 193)
CLSYDTEVLTVEYGAVPIGKLVEEKLNCSVYTVDPN
GYIYTQAIAQWHDRGIQEVFEYQLEDNTIIRATKDH
KFMTEDHQMLPIDEIFERGLELKKCPQPQQ
(SEQ ID NO: 194)
VKIIRRRSLGFQPVYDIGLEQDHNFLLNQGAIASN
>SspPCC7002:/1-129 Synechococcus sp.
PCC 7002
(SEQ ID NO: 195)
CLAGGTPVVTVEYGVLPIQTIVEQELLCHVYSVDAQ
GLIYAQLIEQWHQRGDRLLYEYELENGQMIRATPDH
RFLTTTGELLPIDEIFTQNLDL
(SEQ ID NO: 196)
VKIIRRKFIGHAPTYDIGLSQDMNFLLGQGLIAAN
>AmaMBIC11017:/1-132 Acaryochloris marina
MBIC11017
{SEQ ID NO: 197)
CLSYDTPVLTLEYGWLPIGQVVQEQIECQVFSINER
GHLYTQPIAQWHHRGQQEVFEYTLADGSTIQATAEH
QFMTTDGQMYPVQQIFEEGLSLKQL
(SEQ ID NO: 198)
VKIIQRRSLGLQSVYDIGLAQDHNFVMANGWVAAN
>Mae905:/1-129 Microcystis aeruginosa
DIANCHI905
(SEQ ID NO: 199)
CLGGETLILTEEYGLLPIAKIVSEEVNCTVYSVDKN
GFVYSQPISQWHERGLQEVFEYTLENGQTIQATKDH
KFMTNDGEMLAIDTIFERGLDL
(SEQ ID NO: 200)
VKIISRQSLGRKPVYDIGVEKDHNFLLGNGLIASN
>AciAWQC310F:/1-125 AWQC:
Anabaena circinalis AWQC310F
(SEQ ID NO: 201)
CLSYDTEILTVEYGFLEIGEIVEKQIECKVYTVDSN
GILYTQPIAQWHHRGQQEVYEYLLENGAIIRATKDH
KFMTEAGEMLPIDDIFTQ
(SEQ ID NO: 202)
VKIISRTYVGQANVYD1GVENDHNFVIKNGFVAAN
>AciAWQC131C:/1-125 Anabaena circinalis
AWQC 131C
(SEQ ID NO: 203)
CLSYDTEILTVEYGFLEIGEIVEKQIECRVYTVDSN
GILYTQPIAQWHYRGQQEVYEYLLENGAIIRATKDH
NFMTEAGEMLPIDDIFTQ
(SEQ ID NO: 204)
IKHSRKYVGQANVYDIGYENDHNFVIKNGFVAAN
>CspUCYN:/1-124 Cyanobacterium sp.
UCYN-A2
(SEQ ID NO: 205)
CLSYDTKVLTVEYGPLPIGKVVQENIRCRVYTTNDQ
GLIYTQPIAQVVHNRGKQEIFEYHLDDKTIIRATKE
HQFMTVDHVMMPIDEIFEQ
(SEQ ID NO: 206)
KI1RRKSLGMHEVFD1GLEKDHNFVLSNGLIASN
Pst:/1-129 Planktothrix St147:
st147_cleanDRAFT_c6
(SEQ ID NO: 207)
CLSYDTEVLTVEYGLIPISKIVEEKIECTVYTVNNQ
GYVYTQPIAQWHNRGEQEVFEYYLEDGSVIRATKDH
KFMTVEGQMLPIDEIFEKELDL
(SEQ ID NO: 208)
VKIISRKSLGTQPVYDIGVQEDHNFVLNNGLVASN
>PlaCYA98/1-129: Planktothrix
NIVA-CYA 98
(SEQ ID NO: 209)
CLSYDTEILTVEYGLMPIGKIVKEKIECTVYTVNN
QGWYTQPIAQWHHRGEQEVFEYCLEDGSVIRATKD
HKFMTVQGQMLPIDEIFEKELDL
(SEQ ID NO: 210)
VKIISRKSLGTQPVYDIGVQEDHNFLLNNGLVASN
>FdiUTEX481:/1-137
Fremyella diplosiphon UTEX 481
(SEQ ID NO: 211)
CLSYDTEVLTVEYGLIPIGEIVEKRLECSVYSVDI
NGNVYTQPIAQWHHRGQQEVFEYALEDGSIIRATK
DHKFMTTDGQMLPIDEIFERGLDLLQVPHLPE
(SEQ ID NO: 212)
VKIVTRRAIGAANYTDIGVEQDHNFAIKNGLIAAN
> Pst585:/1-129 Planktothrix sp. 585:
Length = 1586997
(SEQ ID NO: 213)
CLSYDTEILTVEYGLIPISKIVEEKIECTVYTVNN
QGYVYTQPIAQWHNRGEQEVFEYYLEDGSVIRATK
DHKFMTVDGQMLPIDEIFEKELDL
(SEQ ID NO: 214)
VKIISRKSLGTQPVYDIGVQEDHNFVLNNGLVASN
>NpuPCC73102/1-137
(SEQ ID NO: 215)
CLSYETEILTVEYGLLPIGKIVEKRIECTVYSVDN
NGNTYTQPVAQWHDRGEQEVFEYCLEDGSLIRATK
DHKFMTVDGQMLPIDEIFERELDLMRVDNLPN
(SEQ ID NO: 216)
IKIATRKYLGKQNVYDIGVERDHNFALKNGFIASN
>CthPCC7203:/1-137 Chroococcidiopsis
thermalis PCC 7203
(SEQ ID NO: 217)
CLSYDTEILTVEYGAIPIGKIVEERIECTVYSVDN
NGFIYTQPIAQWHNRGQQEVFEYCLEDGSIIRATK
DHKFMTFEGKMLPIDEIFEQELDLKQVKSIQN
(SEQ ID NO: 218)
VKIISRKSLGIQPVYDIGVERDHKFVLKNGLVASN
>NspCCY9414:/1-337 Nodularia spumigena
CCY9414 genome
(SEQ ID NO: 219)
CLSYDTEILTVEYGYIPIGEIVEKAIECSVYSVDN
NGNVYTQPIAQWHNRGEQEVFEYSIEDGSTIRATK
DHKFMTTDGQMLPIDEIFAQELDLLQVHGLPK
(SEQ ID NO: 220)
VKITARKFVGRENVYDIGVERYHNFAIKNGLIASN
>AcyPCC7122:/1-137 Anabaena cyliridrica
PCC 7122
(SEQ ID NO: 221)
CLSYDTEVLTVEYGFIPIGEIVEKRIECSIFSVDK
NGNVYTQPIAQWHNRGRQEIYEYCLDDGSKIRATK
DHKFMTTAGEMLPIDEIFERDLDLLKVEGLPE
(SEQ ID NO: 222)
VKITSRQYLGQADVYDIGVEEDITKFAIKNGFIASN
>CspPCC7507:/1-137 Calothrix sp.
PCC 7507, complete genome
(SEQ ID NO: 223)
CLSYDTEVLTVEYGLLPIGEIVEKGIECRVFSVDN
HGNVYTQPTAQWHNRGQQEVFEYGLDDGSVIRATK
DHKFMTTDGKMLPIDEIFERGLDLLQVQGLPE
(SEQ ID NO: 224)
VKVITRKYIGKENVYDIGVELDHNFAIRNGLVASN
>NspPCC7524:/1-137 Nostoc sp
PCC 7524
(SEQ ID NO: 225)
CLSYDTEDLTVEYGFLPIGEIVEKGIECTVFSVAS
NGIVYTQPIAQWHNRGQQEIFEYCLEDGSIIRATK
DHKFMTQDGQMLPIDEIFACELDLLQVQGLPE
(SEQ ID NO: 226)
VKVVTRKYIGKENVYDIGVERDHNFVIRNGLVASN
>Naz0708:/1-137 ‘Nostoc azollae’ 0708
(SEQ ID NO: 227)
CLSYKTEVLTVEYGLIPIGEIVEKRIECSLFSVDE
NGNIYTQPIAQWHHRGVQEVYEYCLDDGTIIRATK
DFIKFMTTIGEMLPIDEIFERDLNLLQVNGLPT
(SEQ ID NO: 228)
VKIISRQFLGPANVYDIGVAQDHNFAIKNGLIASN
>NspPCC7120:/1-137 Nostoc sp. PCC 7120 DNA
(SEQ ID NO: 229)
CLSYDTEVLTVEYGFVPIGEIVEKGIECSVFSINN
NGIVYTQPIAQWHHRGKQEVFEYCLEDGSIIKATK
DHKFMTQDGKMLPIDEIFEQELDLLQVKGLPE
(SEQ ID NO: 230)
IKIASRKFLGVENVYDIGVRRDHNFFIKNGLIASN
>AvaATCC29413/1-137 Anabaena variabilis
ATCC 29413
(SEQ ID NO: 231)
CLSYDTEVLTVEYGFVPTGEIVDKGTFCSVFSIDS
NGIVYTQPTAQWHHRGKQEVFEYCLEDGSIIKATK
DHKFMTQDGKMLPIDEIFEQELDLLQVKGLPE
(SEQ ID NO: 232)
IKIASRKFLGVENVYDIGVGRDHNFFVKNGLIASN
>PspPCC7327:/1-135 Pleurocapsa sp. PCC 7327.
(SEQ ID NO: 233)
CLSYDTKILTVEYGAMPIGKIVEEQIDCTVYTVNQ
NGFVYTQPIAQWHDRGKQEIFEYCLEDGSIIRATK
DHKFMTTDGCEMLPIDKIFEKGLDLKTINCD
(SEQ ID NO: 234)
VKILSRKSLGIQSVYDIGVEKDHNFLLANGLVASN
>CspPCC7424:/1-135 Cyanothece sp. PCC 7424
(SEQ ID NO: 235)
CLSYETQIMTVEYGLMPIGKIVEEQIDCTVYTVNK
NGFVYTQPIAQWHYRGEQEVFEYCLEDGSTIRATK
DMKFMTTDGQMLPIDEIFEQGLELKQIHLS
(SEQ ID NO: 236)
VKIISRQSLGIQPVYDIGVEKDHNFLISDGLIASN
>CspPCC7822:/1-134 Cyanothece sp.
PCC 7822
(SEQ ID NO: 237)
CLSYDTEILTVEYGPMPIGKTVEEQIECTVYTVDKN
GLVYTQPIAQWHHRGQQEVFEYCLEDGSIIRATKDH
KFMTDDGQMLPIEEIFEKQLELKQIIL
(SEQ ID NO: 238)
VKIISRQLAGNQTVYDLGVEKDHNFLLANGLIASN
>NspPCC7107:/1-137 Nostoc sp. PCC 7107
(SEQ ID NO: 239)
CLSYDTQVLTVEYGLVPIGEIVEKQLECSVFTIDG
HGYVYTQAIAQWHNRGQQEVFEYGLEDGSVIRATK
DHKFMTTDGQMLPIDEIFERELDLLQVQGLRW
(SEQ ID NO: 240)
VKIITRKYIGQANVYDIGVAQDHNFVIENRLIASN
>TboIicb1/1-136 Tolypothrix bouteillei Iicb1
(SEQ ID NO: 241)
CLSYDTEILTVEYGFLPIGKIVEKGIECNVYSVDK
NGNIYTQPIAQWHDRGEQEVFEYCLENGSVIRATK
DHKFMTTSGEMLPIDEIFERGLDLIRVEDLP
(SEQ ID NO: 242)
VKILTRKSIGKQTVYDIGVERDHNFVIKNGSVASN
>Aov:/1-136 Aphanizomenon ovalisporum
DnaE precursor (dnaE) gene
(SEQ ID NO: 243)
CLSADTEILTVEYGFLPIGEIVGKAIECRVYSVDG
NGNIYTQSFAQWHNRGEQEVFEYTLEDGSIIRATK
DFIKFMTTDGEMLPIDEXFARQLDLMQVQGLH
(SEQ ID NO: 244)
VKITARKFVGRENVYDIGVEHHHNFAIKNGLIASN
>OnvPCC7112:/1-137 Oscillatoria nigro-viridis
PCC 7112
(SEQ ID NO: 245)
CLSYDIKILTVEYGPMAIGKIVEEKIECTVYSVDS
NGYIYIQSIAQWHRRGQQEVFEYCLEDGSIIRATK
DHKFMTVGGQMLPIDESFEQGLDLKQINSSSD
(SEQ ID NO: 246)
VKHSRKSLGTQEVYDIGVEREHNFILENSLVASN
>RspPCC7116:/1-135 Rivularia sp. PCC 7116,
complete genome
(SEQ ID NO: 247)
CLSYDTEVLTFEFGLUPIGKIVEEKUDCTVYSVD
VNGNVYSQPSAQWHNRGMQEVFEYELEDGSTIRA
TKDHKFMTVDGEMLAIDEIFEKGLELKRVGIY
(SEQ ID NO: 248)
VKESRKVLKTENVYDIGLEGDHNFIIKDGLIASN
>MspPCC7113:/1-137 Microcoleus sp. PCC 7113,
(SEQ ID NO: 249)
CLSYDSEILTVEYGLMPIGKIVEEGIECTVYSVD
SHGYLYTQPIAQWHHRGQQEVFEYDLEDGSVIRA
TKDHKFMTSEGQMLAIDEIFERGLELKQVKRSQP
(SEQ ID NO: 250)
VKIVRRKSLGIQTVYDIGVERDHNFLLANGLVASN
>ScyPCC7437:/1-137 Stanieria cyanosphaera
PCC 7437
(SEQ ID NO: 251)
CLSYDTEILTVEYGAMPIGKIVKEQIECNVYTVN
QNGFIYPQAIAQWHERGKQEIFEYTLDNGLVIRA
TKDHKFMTIDGQMLPIDEIFERGLELQRINDYSN
(SEQ ID NO: 252)
VKIVSRKSLGKQPVYDIGVTKDHNFLLSNGWASN
>CspPCC6303:/1-137 Calothrix sp. PCC 6303
(SEQ ID NO: 253)
CLSYDTEILTWEYGFLKIGEIVEKQILCSVFSVDE
QGNVYTQPIAQWHNRGLQELFAYQLEDGGVIRATK
DHKFMTTDGQMLAIDEIFERQLDLFQVKGLPE
(SEQ ID NO: 254)
VKIISRKVLKTENVYDIGLEGDHNFIIKDGLIASN
>Cst:/1-134 PCC7202: Cyanobacterium stanieri
PCC 7202
(SEQ ID NO: 255)
CLSYDTEVLTVEYGVLPIGKIVEEQIQCTVYSVDQ
YGFVYTQAIAQWHDRGEQEVFEYELENGATIKATK
DHKMMTSDGQMLPIDQIFEQGLDLFMVSF
(SEQ ID NO: 256)
VKIVKRRSHGIQKVYDIGVAKDHNFLLHNGLVASN
>CspATCC51142:/1-134 Cyanothece sp.
ATCC 51142
(SEQ ID NO: 257)
CLSVDTEILTVEYGPMPIGKIVEENINCTVYTVDP
NGFVYTQAIAQWHYRGEQEIFEYTLEDGATIRATK
DHKFMTMEGKMLPIDEIFENNLDLKQLTL
(SEQ ID NO: 258)
VKIIGRQSLGVQKVYDIGVEKEHNFLLHNGLIASN
>CspPCC8801:/1-134 Cyanothece sp. PCC 8801
(SEQ ID NO: 259)
CLSYDTEILTVEYGAIPIGKVVEENIDCTVYTVDK
NGFVYTQNIAQWHLRGQQEVFEYYLDDGSILRATK
DHQFMTLEGEMLPIHEIFERGLELKKIKI
(SEQ ID NO: 260)
VKIYSYRSLGKQFVYDIGVAQDHNFLLANGSIASN
>Asp:/1-136 Anabaena sp. 90 chromosome
(SEQ ID NO: 261)
CLSYDTEILTVEYGFLEIGEIVEKQIECKVYTIDS
NGMLYTQSIAQWHNRGQQEVYEYLLENGAIIRATK
DHKFMTEAGQMLPIDEIFAQGLDLLQVGVAE
(SEQ ID NO: 262)
VKIVSRTYVGQANVYDIGVESDHNFVIKNGFIASN
>Aha:/1-137 Aphanothece halophytica
(SEQ ID NO: 263)
CLSYDTEIWTVEYGAMPIGKIVEEKIECSVYTVDE
NGFVYTQPIAQWHPRGQQEIIEYTLEDGRKIRATK
DHKMMTESGEMLPIEEIFQRELDLKVETFHEM
(SEQ ID NO: 264)
VKIIKRQSLGRQNVYDVCVETDHNFVLANGCVASN
>HspPCC7418:/1-137 Halothece sp. PCC 7418
(SEQ ID NO: 265)
CLSYDTEIWTVEYGAMPIGKIVEEKIECSVYTVDE
NGFVYTQPIAQWHPRGQQEIIEYTLEDGRKIRATK
DHKMMTESGEMLPIEEIFQRELDLKVETFHEM
(SEQ ID NO: 266)
VKIIKRQSLGRQNVYDIGVETDHNFVLANGCVASN
>CapPCCl 0605:/1-137 Cyanobacterium aponinum
PCC 10605
(SEQ ID NO: 267)
CLSYDTEILTVEYGAISIGKIVEEKINCQVYSVDK
NGFIYTQNIAQWHDRGSQELFEYELEDGRIIKATK
DHKMMTKDGQMLAINDIFEQELELYSVDDMGV
(SEQ ID NO: 268)
VKIVKRRSLGVQPVYDIGVEKDHNFILANGLVASN
>Cat:/1-133 Candidatus Atelocyanobacterium
thalassa isolate
(SEQ ID NO: 269)
CLSYDTKVLTVEYGPLPIGKVVQENIRCRVYTTND
QGLTYTQPIAQWHNRGKQEIFEYHLDDKTORATKE
HQFMTVDHVMMPIDEIFEQGLELKKIK
(SEQ ID NO: 270)
LKIIRRKSLGMHEVFDIGLEKDIINFVLSNGLIASN
>Oli:/1-137 Oscillatoria limnetica
‘Solar Lake’ DnaE precursor
(SEQ ID NO: 271)
CLSYNTEVLTVEYGPLPTGKLIVDEQIHCRVYSVD
ENGFVYTQAIAQWHDRGYQEIFAYELADGSVIRAT
KDHQFMTEDGQMFPIDEIWEKGLDLKKLFIVQD
(SEQ ID NO: 272)
VKIVRRQSLGVQNVYDIGVEKDHNFLLASGEIASN
>Cen:/1-137 Cyanobacterium endosymbiont
of Epithemia turgida
(SEQ ID NO: 273)
CLSYDTEVLTVEYGAIPIGRMVEESLDCTVYTVDK
NGFVYTQSIQQWMSRGQQEIFEYCFEDGSTIRATK
DHKFMTAEGKMSSTITDIFEQGLELKKIIPWSG
(SEQ ID NO: 274)
AKIISCKSLGKQVYDIGVVQDHNFLLANGVVASN
>SspPCC7502:/1-133 Synechococcus sp. PCC 7502
(SEQ ID NO: 275)
CLGYDTPVLTVEYGFMPIGKIVEEKIQCHVYSVDQ
NGLVFTQAIAQWHNRGQQEVWEYNLDNGDIVRATK
DHKFMTIDGQMLPINQIFEQGLELKVIA
(SEQ ID NO: 276)
VKIVSCKPLRVQTVYDIGVEKDHNFILDNGLVASN
>CspUCYN:/1-124 Cyanobacterium sp. UCYN-A2
(SEQ ID NO: 277)
CLSYDTKVLTVEYGPLPIGKVVQENIRCRVYTTNDQ
GLIYTQPIAQWHNRGKQEIFEYHLDDKTIIRATKEH
QFMTVDHVMMPIDEIFEQ
(SEQ ID NO: 278)
KIIRRKSLGMHEVFDIGLEKDHNFVLSNGLIASN
>Pst:/1-129 Planktothrix st147:
st_147_cleanDRAFT_c6
(SEQ ID NO: 279)
CLSYDTEVLTVEYGLIPISKIVEEKIECTVYTVNNG
YVYTQPIAQWHNRGEQEVFEYYLEDGSVIRATKDHK
FMTVEGQMLPIDEIFEKELDL
(SEQ ID NO: 280)
VKIISRKSLGTQPVYDIGVQEDHNFVLNNGIVASN
>PlaCYA98/1-129: Planktothrix NIVA-CYA 98
(SEQ ID NO: 281)
CLSYDTEILTVEYGLMPIGKIVKEKIECTVYTVNN
QGYVYTQPIAQWHHRGEQEVFEYCLEDGSVIRATK
DHKFMTVQGQMLPIDEIFEKELDL
(SEQ ID NO: 282)
VKIISRKSLGTQPVYDIGVQEDHNFLLNNGLVASN
>Pst585:/1-129 Planktothrix sp.
585: Length = 1586997
(SEQ ID NO: 283)
GLSYDTEILTVEYGLIPISKIVEEKIECTVYTVNN
QGYVYTQPIAQWHNRGEQEVFEYYLEDGSVIRATK
DHKFMTVDGQMLPIDEIFEKELDL
(SEQ ID NO: 284)
VKIISRKSLGTQPVYDIGVQEDHNFVLNNGLVASN
>CspPCC8802/1-134: Cyanothece sp. PCC 8802
(SEQ ID NO: 285)
CLSYDTEILTVEYGAIPIGKVVEENIDCTYYTVDK
NGFVYTQNIAQWHLRGQQEVFEYYLDDGSILRATK
DHQFMTLEGEMLPIHEIFERGLELKKIKI
(SEQ ID NO: 286)
VKIVSYRSLGKQFVYDIGVAQDHNFLLANGSIASN
>CfrPCC6912:/1-137 Chlorogloeposis fritschii
PCC 6912
(SEQ ID NO: 287)
CLSYDTAILTVEYGFLPIGEIVEKGIECTVYTVDS
NGYIYTQPIAQWHNRGEQELFEYSLEDGSIIRATK
DHKFMTIDGQMLPIDEIFARKLELMQVKGLPE
(SEQ ID NO: 288)
VKIIAKKSLGTQNYYDIGVERDHNFVIKNGLVASN
>CspATC51472:/1-132 Cyanothece sp. ATCC 51472
(SEQ ID NO: 289)
CLSYDTEILTVEYGPMPIGKIVEENINCTVYTVDP
NGFVYTQAIAQWHYRGEQEIFEYYLEDGATIRATK
DHKFMTMEGKMLPIDEIFENNLDLKQL
(SEQ ID NO: 290)
VKHGRQSLGVQKVYDIGVEKEHNFLLHNGLIASN
>Lma:/1-132 Lyngbya majuscula
(SEQ ID NO: 291)
CLSYDTEIITVEYGPIAIGEIVEKGIPCTVYSVDS
NGYVYTQPIAQWHNRGEQEVFEYTLDDGSVIRATK
DHKFMTIDGQMLPIDEIFEGGLELKQL
(SEQ ID NO: 292)
VKIISRKSLGTQPVYDIGVKDDHNFILANGMVASN
>CspESFC/1-137
(SEQ ID NO: 293)
CLSYDTEVLTVEYGAVPIGKLVEEKLNCSVYIVDP
NGYIYTQAIAQWHDRGIQEVFEYQLEDNTIIRATK
DHKFMTEDHQMLPIDEIFERGLELKKCPQPQQ
(SEQ ID NO: 294)
VKIIRRRSLGFQPVYDSGLEQDHNFLLNQGAIASN
>Mae905:/1-129 Alicrocystis aeruginosa
DIANCHI905
(SEQ ID NO: 295)
CLGGETLILTEEYGLLPIAKIVSEEVNCTVYSVDK
NGFVYSQPISQWHERGLQEVFEYTLENGQTIQATK
DHKFMTNDGEMLAIDTIFERGLDL
(SEQ ID NO: 296)
VKIISRQSLGRKPVYDIGVEKDHNFLLGNGLIASN
>RlaKORDI51-2:/1-137 Rubidibacter lacunae
KORDI 51-2
(SEQ ID NO: 297)
CLSYDTEVLIYEYGPLAIGTIVSERLACTVYIVDR
SGFLYAQAISQWHERGRQDVFEYALDNGMTIRATK
DHKLMTADGQMVAIDDIFTQGLTLKAIDTAAF
(SEQ ID NO: 298)
MKIVSRKSLGVQHVYDIGVARDHNFLLANGAIASN
>CfrPCC9212/1-136 Chlorogloeopsis fritschii
PCC 9212
(SEQ ID NO: 299)
CLSYDTAILTVEYGFLPIGEIVEKGIECTVYTVDS
NGYIYTQPIAQWHNRGEQELFEYSLEDGSIIRATK
DHKFMTIDGQMLPIDEIFARKLELMQVKGLP
(SEQ ID NO: 300)
VKDAKKSLGTQNVYDIGVERDHNFVIKNGLVASN
>RinHH01:/1-137 Richelia intracellularis
HH0.1 WGS project
(SEQ ID NO: 301)
CLSYDTQILTVEHGPMS1GEIVEKCLECHVYTVNK
NGNICIQTITQWHFRGEQEIFEYELEDGSFIQATK
DHKFMTTTGEMLPIHEIFTNGLEILQLSKSLL
(SEQ ID NO: 302)
VKILARKSLGTQKVYDIGVNDDHNFALSNSFIASN
>GhePCC6308:/1-133 Geminocystis herdmanii
PCC 6308
(SEQ ID NO: 303)
CLSYDTEVLTVEFGAIPMGKIVEERLNCQVYSVDK
NGFIYTQNIAQWHDRGVQEVFEYELEDGRIIKATK
DHKMMIENCEMVEIDRIFEEGLELFEVN
(SEQ ID NO: 304)
VKILKRRSISSQQVYDIGVEKDHNFLLANGLVASN
>SsuPCC9445:/1-131 Spirulina subsalsa
PCC 9445
(SEQ ID NO: 305)
CLSYDTKIITVEYGAIAIGTIVEQGLHCHVYSVDP
NGFIYTQPIAQWHQRGEQEVFAYTLENGSIIQATK
DHKFMTQQGKMLPIDTIFEQGLDLLQV
(SEQ ID NO: 306)
KIIKRTSLGVRPVYDIGVIQDHNFLLENGLVASN
>MaePCC9807:/1-135 Microcystis aeruginosa 9807
(SEQ ID NO: 307)
CLGGETLILTEEYGLLPIAKIVSEEINCTVYSVDK
NGFIYSQPISQWHERGLQEVFEYTLENGQTIQATKD
HKFMTSDGEMLAIDTIFERGLDLKSSDFS
(SEQ ID NO: 308)
VKIISRQFLGRKPVYDIGVEKDHNFLLGNGLIASN
>MspGII:/1-130 Myxosarcina sp. GI1 contig_13
(SEQ ID NO: 309)
CLSYDTEVLTLKYGALPIGEIVEKRINCHVYTRAE
SGFFYIQSIEQWHDRGEQEVFEYTLENGATIKATK
DHKFMTSGGQMLPIDEIFERGLDLL
(SEQ ID NO: 310)
VKIVSRKSLGKQPVYDLGVAKDHNFLLANGTVASN
>ShoPCC7110:/1-136 Scytonema hofmanni
PCC 7110 contig00136
(SEQ ID NO: 311)
CLSYDTEVLTAEYGFLPIGKIVEKAIECTVYSVD
NDGNIYTQPIAQWMDRGQQEVFEYSLDDGSVIRA
TCDHKFMTTCGQMLPIDEFFERCLDLMRIDSLP
(SEQ ID NO: 312)
VKILTRKSIGKQTVYDIGVERDHNFVIKNGLVASN
>WinUHHT291/1-136 Westiella intricata
UHHT-29-1
(SEQ ID NO: 313)
CLSYDTEILTVEYGFLPIGEIVEKRIECTVYTVD
TNGYVYTQAIAQWHNRGEQEVFEYALEDGSMRAT
KDHKFMTSEGQMLPIDEIFVKGLDLLQVQGLP
(SEQ ID NO: 314)
VKIITRKFLGIQNVYD1GVEQNHNFVIKNGLVASN
>FspPCC9605:/1-136 Fischerella sp.
PCC 9605 FIS9605DRAFT
(SEQ ID NO: 315)
CLSYDTETLTVEYGFLPIGEIVEKGIECTVYTVDN
NGNVYTQTIAQWHNRGQQEVFEYCLEDGSVIRATK
DHKFMTTDGQMLPIDEIFARGLDLLQVKNLP
(SEQ ID NO: 316)
VKIVTRRPLGTQNVYDIGVESDHNFVIKNGLVASN
>MrePCC10914:/1-137 Mastigocladopsis repens
PCC 10914
(SEQ ID NO: 317)
CLSYDTEVLTVEYGFLPIGEIVEKSIECSVYTVDS
NGNVYTQPIAQWHNRGQQEVFEYCLEDGSIIRATK
DHKFMTEIGQMLPIDEIFERGLELMKIQGLPE
(SEQ ID NO: 318)
AKIITRKSLGTQNVYDIGVERDHNFVTRDGFIASN
>ShoUTEX2349:/1-137 [Scytonema hofmanni]
UTEX 2349
(SEQ ID NO: 319)
CLSYNSEVLTVEYGFLPIGKIVEKGIECSVYSVDS
YGKIYTQVIAQWHNRGQQEVFEYCLEDGTIIQAT
KDHKFMTVDGQMLPIDEIFERGLDLMQVQGLPD
(SEQ ID NO: 320)
VKIITRKSLGTQNVYDIGVSSDHNFVMKNGLIASN
>AspPCC7108:/1-137 Anabaena sp.
PCC 7108 Ana7108scaffold_2_Cont3
(SEQ ID NO: 321)
CLSSDTEVLTVEYGLIPIGEHEKRIDCSVFSVDKN
GNIYTQPIAQWHDRGIQELYEYCLDDGSTIRATKD
HKFMTTAGEMLPIDEIFERGLDLLKVMNLPQ
(SEQ ID NO: 322)
VKIITRNYVGKENVYDIGVERDHNFAIKNGLIASN
>FspPCC9339:/1-137 Fischerella sp.
PCC 9339 PCC9339DRAFT
(SEQ ID NO: 323)
CLSYDTEVLTVEYGFLPIGEIVEKRIECTVYTVD
HNGYVYTQPIAQWHNRGYQEVFEYGLEDSVIRAT
KDHKFMTSEGQMLPIDEIFARELDLLQVTGLVN
(SEQ ID NO: 324)
VKIVTRRLLGIQNVYDIGVEQNHNFVIKNGLVASN
>Csp336:/1-137 Calothrix sp. 336/3
(SEQ ID NO: 325)
CLSYDIEIFTVEYGFLPIGEIVEKRLECTVLTVD
NHGNIYSQPIAQWHHRGQQQIYEYGLEDGSVIRA
TKDHKFMTTDGQMLPIDEIFERGLDLLQVTNLDN
(SEQ ID NO: 326)
VRVITRKLADTENVTDIGVENHHNFLIKNGLVASN
>FthPCC7521:/1-136 Fischerellai hermalis
PCC 7521
(SEQ ID NO: 327)
CLSYETEILTVEYGFLPIGEIVEKRIECSVYTVDN
NGYVCTQPIAQWHNRGYQEVFEYGLEDGSVIRATK
DHKFMTIDRQMLPIDEIFARGLDLLQVTGCP
(SEQ ID NO: 328)
VKIITRKSLGTQNVYDIGVEQNHNFVIKNGLVASN
>CyaPCC7702/1-137 cyanobacterium
PCC 7702 CHl7702
(SEQ ID NO: 329)
CLSYDTEILTVEYGFLSIGEIVEKEIECTVYIYDS
NGYIYTQPIAQWHEQGEQEIFEYSLEDGSTIRATK
DHKFMTIECEMLPIDQIFARQLDLMQITGLPQ
(SEQ ID NO: 330)
VKISTKKSLGKQKVYDIGVVRDHNFIIKNGFVASN
>FspPCC9431:/1-136 Fischerella sp.
PCC 9433
(SEQ ID NO: 331)
CLSYDTEVLTVEYGFLPIGEIVEKRIECTVYTVDT
NGYVYTQAIAQWHNRDEQEVFEYALEDGSTIRATR
DHKFMTSEGQMLPIDEIFAKGLDLLQVQGLP
(SEQ ID NO: 332)
VKIVTRKFLGIQNVYDTGVEQNHNFVTKNGLVASN
>FmuPCC7414:/1-137 Fischerella muscicola
PCC 7414
(SEQ ID NO: 333)
CLSYETEILTVEYGFLPIGEIVEKRIECSVYTVDN
NGYVCTQTIAQWHNRGYQEVFEYGLEDGSVIRATK
DHKFMTIDRQMLPIDEIFARGLDLLQVKGLPE
(SEQ ID NO: 334)
VKHTRQSLGTQNVYDIGVEQNHNFVIKNGLVASN
>FmuPCC73103:/1-137 Fischerella muscicola
SAG 1427-1 = PCC 73103
(SEQ ID NO: 335)
CLSYDTEVLTCEYGFLPIGEIVEKTIECNVFTVDS
NCYVYTQPIAQWHNRGYQEVFEYGLEDGSVIRATK
DHKFMTSEGKMLPIDEIFARELDLLQVTGLIN
(SEQ ID NO: 336)
VKIVTRKFLGIQNVYDIGVEQNHNFVIKNGLVASN
>Lae:/1-137 Lyngbya aestuarii BL J
laest3.contig.3
(SEQ ID NO: 337)
CLSYDTEILTVEYGAIPIGKVVDEKIECTVYSVDK
NGLIYTQPIAQWHNRGKQEVFEYSLEDGSTIRATK
DHICFMTMDNQMLPIDEILEKGLELKQVNADSV
(SEQ ID NO: 338)
VKIVSRKSLDSQTVYDIGVETDHNFLLANGSVASN
>Lsp:/1-137 Leptolyngbya sp. JSC-1
(SEQ ID NO: 339)
CLSYDTEILTVEYGALPIGKIVENQMICSVYSIDN
NGYIYIQPIAQWHNRGQQEVFEYILEDGSIIRSTK
DHKFMTKGGEMLPIDEIFERGLELAQVTRLEQ
(SEQ ID NO: 340)
VKHSRRSVGVQSYTDIGVKQDHNFFLRNGLIASN
>CwaWH8501:/1-137 Crocosphaera watsonii
WH8501
(SEQ ID NO: 341)
CLSYDTEILTVEYGAMYIGKIVEENINCTVYTVDK
NGFVYTQTIAQWHNRGEQEIFEYDLEDGSKIKATK
DHKFMTIDGEMLPIDEIFEKNLDLKQVVSHPD
(SEQ ID NO: 342)
VKIIGCRSLGTQKVYDIGVEKDHNFLLANGSIASN
>CchPCC7420:/1-135 Coleofasciculus
chthonoplastes PCC 7420
(SEQ ID NO: 343)
CLSYDTQILTVEYGAVAIGEIVEKQIECTVYSVDE
NGYVYTQPIAQWHNRGEQEVFEYLLEDGATIRATK
DHKFMTDEDQMLPIDQIFEQGLELKQVEVL
(SEQ ID NO: 344)
VKIIGRKPLGTQPVYDIGVERDHNFLLFNGSVASN
>CspPCC6712/1-133
(SEQ ID NO: 345)
CLSYDTEVLTVEYGAIPIGKIVEEKIACNVYSVDK
NGFVYTQPIAQYHDRCHQEVFEYRLENGSVIRATK
DHKMMTADGQMLPIDEIFKQNLDLKQLN
(SEQ ID NO: 346)
VKIISRQSLGKQSVFDIGVAKDHNFLLANGLVASN
>Rbr:/1-137 Raphidiopsis brookii D9 D9_5,
(SEQ ID NO: 347)
CSYETEVLTLEYGFLPIGFIVDKQMVCTVFSVND
SGNVYTQPIGQWHDRGVQELYEYCLDDGSTIRAT
KDHKFMTTQGEMVPIDEIFHQGWELVQVSGTMN
(SEQ ID NO: 348)
VKIVSRRYLGKADVYDIGVAKDHNFIIKNGLVASN
>CspCCy0110:/1-134 Cyanothece sp.
CCY0110 1101676644604
(SEQ ID NO: 349)
CLSYDTEILTVEYGPMPIGKIVEENINCSVYTVN
KNGFWTQSIAQWHHRGEQEVFEYYLEDGETIRAT
KDHKFMTTEGKMLPIDEIFENNLDLKKLTV
(SEQ ID NO: 350)
VKIIERRSLGKQNVYDIGVERDHNFLLSNNLIASN
>XspPCC7305:/1-135 Xenococcus sp.
PCC 7305
(SEQ ID NO: 351)
CLSADTEVLTVEYGAISIGKIVEERIECTVYSVDA
NGFVYTQEIAQWHNRGEQEVFEYMLDDGSV1RATK
DHKLMTIDGQMVAIDEIFSQGLELKQVLGL
(SEQ ID NO: 352)
VKIVSRKSLGTQTVYDLGVARDHNFLLANGTVASN
>PspPCC7319:/1-135 Pleurocapsa sp.
PCC 7319
(SEQ ID NO: 353)
CLSYDTEIYTVEYGALPIGKIVESRIKCTVLTVDK
NGLVYSQPIVQWHDRGIQEVFEYTLDNGATIRATK
DHKFMTVEGQMLPIDEIFELGLELKEIQQF
(SEQ ID NO: 354)
VKIISRQSLGKQSVYDIGVAKDHNFLLANGMVASN
>CraCS505:/1-137 Cylindrospermopsis
raciborskii CS-505
(SEQ ID NO: 355)
CLSYETEVITLEYGFVPIGEIVNKQMVCTVFSLNDS
GNVYTQPSGQWMDRGVQDLYEYCLDDGSTIRATKDH
KFMTTQGEMVPIDEIFHQGWELVQVSGISK
(SEQ ID NO: 356)
VKIVSRRYLGKADVYDIGVAKDHNFIIKNGLVASN
>MaePCC7806:/1-135 Microcystis
aeruginosa PCC 7806
(SEQ ID NO: 357)
CLGGETLILTEEYGLLPIAKIVSEEVNCTVYSVDK
NGFVYSQPISQWHERGLQEVFEYTLENGQTIQATK
DHKFMTNDGEMLAIDTIFERGLDLKSSDFS
(SEQ ID NO: 358)
VKIISRQSLGRKPVYDIGVEKDHNFLLGNGLIASN
>MaeNIES843:/1-135 Microcystis
aeruginosa NIES-843 DNA
(SEQ ID NO: 359)
CLGGETLILTEFYGLLPIAKIVSEEINCTVYTVDQN
GFVTSQPISQWHERGLQEVFEYTLENGQTIQATKDH
KFMTSDGEMLAIDTIFERGLDLKSSDFS
(SEQ ID NO: 360)
VKHGRQSLGRKPVYDIGVEKDHNFLLGNGLIASN

FIG. 1 shows an alignment and a computer-generated model of the design of the Cfa split intein according to an embodiment of the invention. Panel A shows a sequence alignment of Npu DnaE and Cfa DnaE. The sequences share 82% identity with the differences (underlined, cyan) evenly distributed through the primary sequence. Catalytic residues and second shell ‘accelerator’ residues are shown in caret, orange and asterisk, green, respectively. Panel B shows the same residues highlighted in panel a mapped on to the Npu structure (pdb=4kl5).

The Cfa intein has high sequence similarity to Npu (82%), and the non-identical residues are spread throughout the 3D structure of the protein.

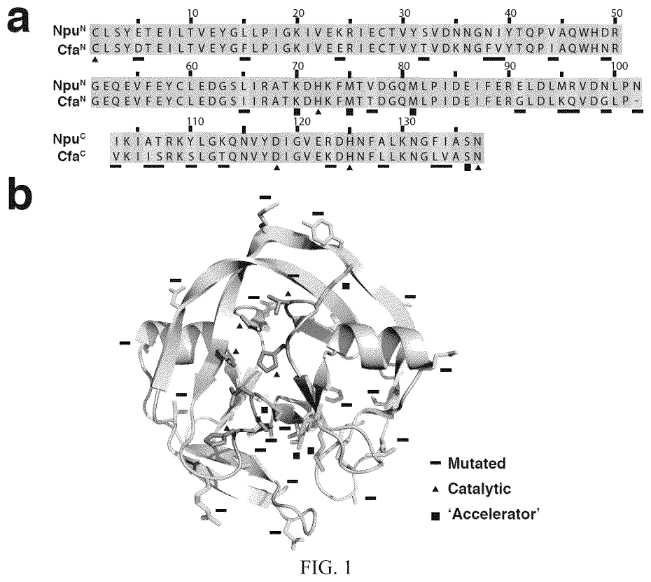

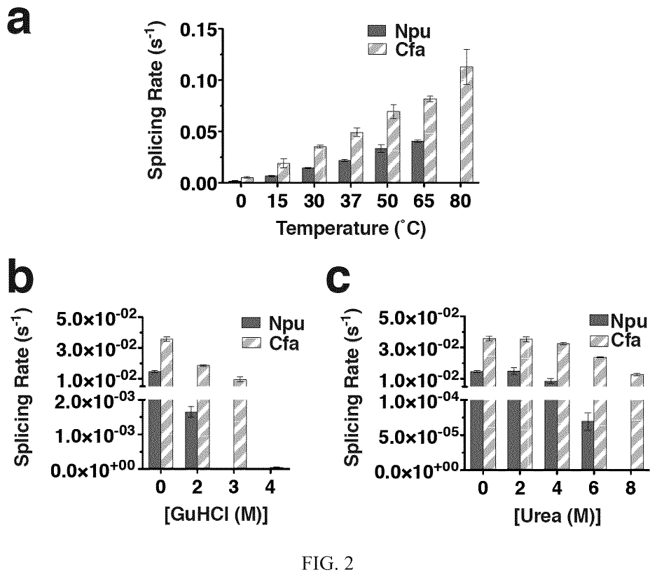

Cfa intein fragments fused to model exteins were generate and their PTS activity was measured using the aforementioned in vitro assay (FIG. 2). This revealed that the Cfa intein splices 2.5 fold faster at 30° C. than Npu (t_{1/2} 20 s vs. 50 s), a notable enhancement in activity since the latter is the fastest characterized DnaE split intein (FIG. 2A). This accelerated rate manifests itself both in branch formation (3-fold increase) and branch resolution (2-fold increase). In line with parent DnaE inteins, Cfa retains the preference for a bulky hydrophobic residue at the +2 position of the C-extein. Strikingly, Cfa shows an increased splicing rate as a function of temperature and is consistently faster than Npu (FIG. 2A). The Cfa intein even maintains activity at 80° C., albeit with reduced yield of splice products, while Npu is inactive at this temperature. These results demonstrate that consensus engineering is effective in producing an intein that is highly active across a broad range of temperatures.

Applications of PTS typically require fission of a target protein and fusion of the resulting fragments to the appropriate split intein segments.¹ As a consequence, the solubility of these fusion proteins can sometimes be poor. Because protein denaturants such as guanidine hydrochloride (GuHCl) and urea are frequently used to keep these less soluble fragments in solution, the ability of Cfa to splice in the presence of these chaotropic agents was tested. Cfa intein was found to splice in the presence of up to 4M GuHCl (with little decrease in activity seen up to 3M), while no activity was observed for Npu in ≥3M GuHCl (FIG. 2B). Remarkably, the splicing of Cfa is largely unaffected up to 8M urea, while splicing of Npu falls off dramatically above 4M urea (FIG. 2C).

FIG. 2 shows graphs showing the characterization of the Cfa intein according to an embodiment of the invention. In Panel A, splicing rates for Cfa and Npu as a function of temperature are shown. Npu is inactive at 80° C. (error=SD (n=3)). In Panels B and C, splicing rates for Cfa and Npu as a function of added chaotrope are shown. Npu is inactive in 3M GuHCl or 8M Urea. Note, Cfa has residual activity in 4M GuHCl (k=7×10⁻⁵) (error=SD (n=3)).

The unprecedented and unexpected tolerance of Cfa to high concentrations of GuHCl and urea suggests the intein might retain activity directly following chaotropic extraction of insoluble proteins from bacterial inclusion bodies, thereby expediting PTS-based studies. Accordingly, the model fusion protein, His₆-Sumo-Cfa^{N}, was overexpressed in E. coli cells and extracted the protein from inclusion bodies with 6M urea. The protein was purified from this extract by nickel affinity chromatography and then directly, and efficiently, modified by PTS under denaturing conditions, i.e. without the need for any intervening refolding steps. In general, it is expected that the robust activity of Cfa in the presence of chaotropic agents will prove useful when working with protein fragments that demonstrate poor solubility under native conditions.

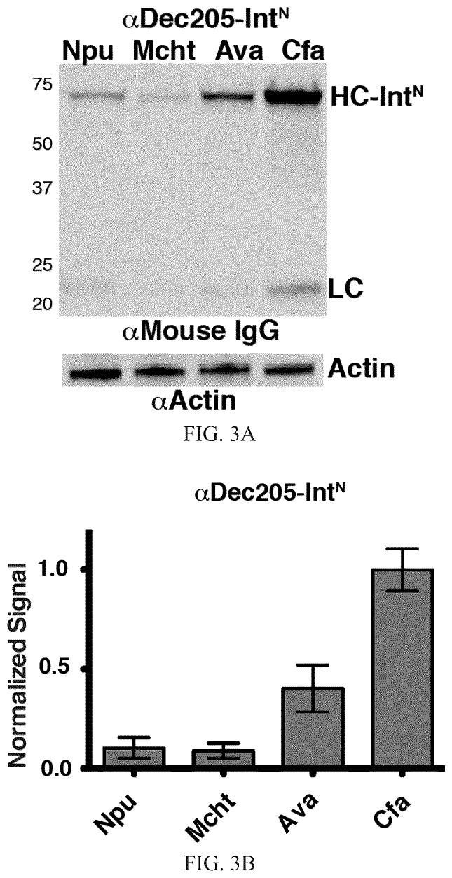

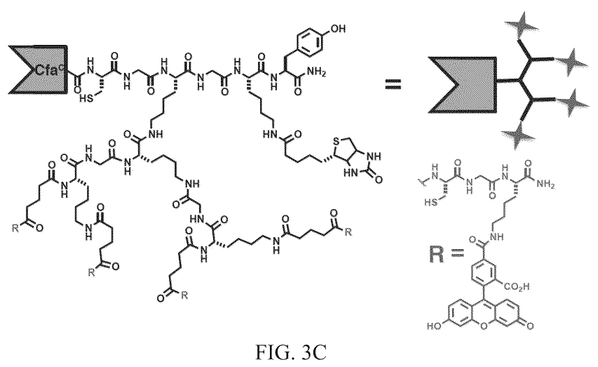

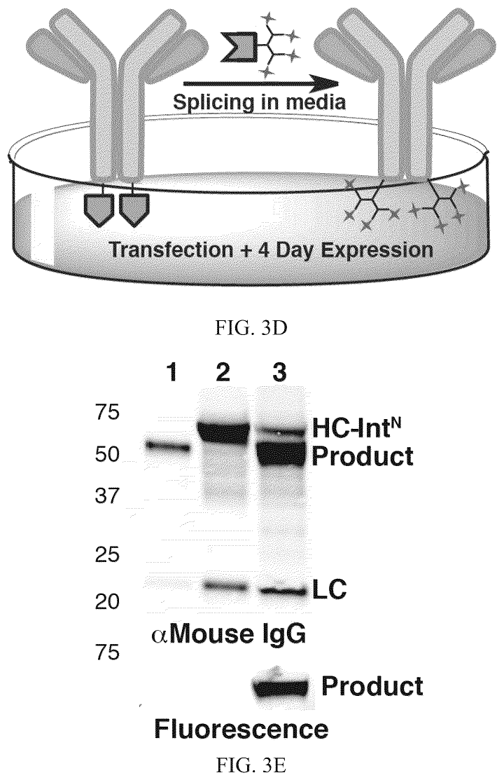

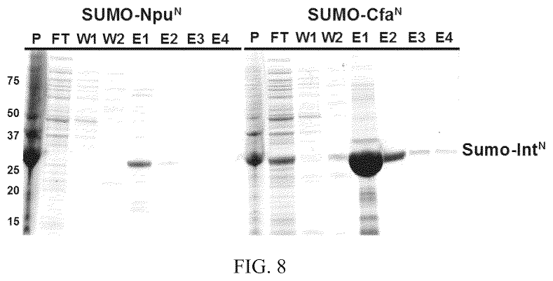

Fusing a protein of interest to a split intein can result in a marked reduction in cellular expression levels compared to the protein alone.⁶ This situation is more frequently encountered for fusions to N-inteins than to C-inteins, which is likely due to the larger size of the former and their partially folded state.¹⁸ It was therefore investigated whether the improved thermal and chaotropic stability of Cfa would translate to increased expression levels of Cfa^{N} fusions. Indeed, model studies in E. coli revealed a significant (30-fold) increase in soluble protein expression for a Cfa^{N} fusion compared to the corresponding Npu^{N} fusion (FIG. 8). Given this result, it was investigated whether Cfa^{N} fusions would also exhibit increased protein expression levels in mammalian cells. In particular, intein fusions to the heavy chain (HC) of monoclonal antibodies (mAbs) have emerged as a powerful tool for site-specific conjugation of synthetic cargoes.¹⁹⁻²¹ The expression levels in HEK293 cells of a mAb (αDec205) as a function of the N-intein fused to its HC was explored. Consistent with the bacterial expression results, production of the HC-Cfa^{N} fusion was significantly higher than for the other inteins examined; for example, the secreted levels of the mAb-Cfa construct were ~10-fold higher than for the corresponding Npu fusion (FIGS. 3A and 3B). Importantly, mAb-Cfa retained PTS activity and could be site-specifically modified with a synthetic peptide by splicing directly in the growth medium following the four-day expression at 37° C.

FIG. 8 is an SDS-PAGE analysis of test expression of His₆-SUMO-Npu^{N} and His₆-SUMO-Cfa^{N}. Coomassie brilliant blue stained gel from a 4 mL column volume (CV) Ni-NTA purification of the soluble fraction of 1 L of E. coli culture. Lanes correspond to (P) the inclusion body pellet, (FT) flow through of batch bound Ni-NTA solution, (W1) a 5CV wash with 5 mM imidazole, (W2) a 5CV wash of 25 mM imidazole, (E1-E4) and four 1.5CV elutions of 250 mM imidazole.

Finally, to further explore the utility of the Cfa intein in the context of antibody conjugation, whether the PTS system could be used to attach multiple copies of a synthetic cargo to the heavy chain of the mAb was investigated. Accordingly, semisynthesis was used to prepare a construct in which the C-terminal half of Cfa (Cfa^{C}) was fused to a C-extein containing a dendrimeric scaffold allowing multimeric attachment of cargo, in this case fluorescein (FIG. 3C). This dendritic cargo was successfully linked to the αDec205 antibody via Cfa-mediated PTS, again performed directly in situ within the cellular growth medium (FIGS. 3D and 3E). This represents the first time that PTS has been used to attach a branched extein construct to a target protein, highlighting the potential of the system for manipulating the payload quantity of antibody drug conjugates.²

FIGS. 3A to 3E show expression and modification of a mouse monoclonal antibody using the Cfa intein according to an embodiment of the invention. FIG. 3A shows test expression in HEK293T cells of various IntN homologues (Npu, Mcht, Ava and Cfa) fused to the C-terminus of the heavy chain of a mouse uDec205 monoclonal antibody. Top: Western blot analysis (αMouse IgG) of antibody levels present in the medium following the 96 hour expression. Bottom: α-actin western blot of cell lysate as a loading control. FIG. 3B shows quantification of normalized expression yield by densitometry of αDEC205 HC-IntN signal in panel A (error=SD (n=4)). FIG. 3C shows the structure of the CfaC-dendrimer construct used in PTS reactions with the αDEC205 HC-IntN fusion. For simplicity, the CfaC peptide sequence is depicted symbolically in green (as a rectangle with a triangular cut-out on the left). FIG. 3D is a schematic of the in situ PTS approach used to modify the HC of a mAb with a multivalent cargo. FIG. 3E is an SDS-PAGE analysis of PTS reaction. Lane 1: Wild type mouse αDEC205 mAB. Lane 2: Mouse αDEC205-CfaN mAB fusion. Lane 3: addition of the CfaC-dendrimer to the media containing the αDEC205-CfaN mAB. The splicing reaction was analyzed by fluorescence (bottom) and western blot (top, αMouse IgG).

The discovery of fast split inteins has revolutionized the applications of protein trans splicing. The remarkable robustness of the Cfa intein described in this study should extend the utility of many of these technologies by allowing PTS to be performed in a broader range of reaction conditions. Moreover, the ability of Cfa to increase the expression yields of N-intein fusions should encourage further use of split inteins for protein semisynthesis. The activity-guided approach we use to engineer this intein may be applied to other intein families or act as a general strategy for the refinement of multiple sequence alignments used for consensus engineering.

### Materials and Methods

### Materials

Oligonucleotides and synthetic genes were purchased from Integrated DNA Technologies (Coralville, IA). The QuickChange XL II site directed mutagenesis kit and Pfu Ultra II Hotsart fusion polymerase were purchased from Agilent (La Jolla, CA). All restriction enzymes and 2× Gibson Assembly Master Mix were purchased from New England Biolabs (Ipswich, MA). “In-house” high-competency cells used for cloning and protein expression were generated from One Shot B121 (DE3) chemically competent E. coli and sub-cloning efficiency DH5u competent cells purchased from Invitrogen (Carlsbad, CA). Dulbecco's Modified Eagle Medium (DMEM), Lipofectamine 2000, and low IgG fetal bovine serum were purchased from Invitrogen as well. DNA purification kits were purchased from Qiagen (Valencia, CA). All plasmids were sequenced by GENEWIZ (South Plainfield, NJ). N,N-diisopropylethylamine (DIPEA), Luria Bertani (LB) media, and all buffering salts were purchased from Fisher Scientific (Pittsburgh, PA). Dimethylformamide (DMF), dichloromethane (DCM), Coomassie brilliant blue, triisopropylsilane (TIS), β-Mercaptoethanol (BME), DL-dithiothreitol (DTT), sodium 2-mercaptoethanesulfonate (MESNa), tetrakis(triphenylphosphine)palladium(0) (Pd(PPh₃)₄), and 5(6)-carboxyfluorescein were purchased from Sigma-Aldrich (Milwaukee, WI) and used without further purification. Tris(2-carboxyethyl)phosphine hydrochloride (TCEP) and isopropyl-β-D-thiogalaetopyranoside (IPTG) were purchased from Gold Biotechnology (St. Louis, MO). The protease inhibitor used was the Roche Complete Protease Inhibitor (Roche, Branchburg, NJ). Nickel-nitrilotriacetic acid (Ni-NTA) resin was purchased from Thermo scientific (Rockford, IL). Fmoc amino acids were purchased from Novabiochem (Darmstadt, Germany) or Bachem (Torrance, CA). (7-Azabenzotriazol-1-yloxy)tripyrrolidinophosphonium hexafluorophosphate (PyAOP) and O-(Benzotriazol-1-yl)-N,N,N′,N′-tetramethyluronium hexafluorophosphate (HBTU) were purchased from Genscript (Piscataway, NJ). Rink Amide-ChemMatrix resin was purchased from Biotage (Charlotte, NC). Trifluoroacetic acid (TFA) was purchased from Halocarbon (North Augusta, SC). Immun-blot PVDF membrane (0.2 μm) and Criterion XT Bis-Tris gels (12% polyacrylamide) were purchased from Bio-Rad (Hercules, CA). MES-SDS running buffer was purchased from Boston Bioproducts (Ashland, MA). Anti-Mouse IgG secondary antibody (Licor mouse 800) and Mouse αActin primary antibody were purchased from Li—COR biotechnology (Lincoln, NE).

### Equipment

Analytical RP-HPLC was performed on Hewlett-Packard 1100 and 1200 series instruments equipped with a C₁₈ Vydac column (5 μm, 4.6×150 mm) at a flow rate of 1 mL/min. Preparative RP-HPLC was performed on a Waters prep LC system comprised of a Waters 2545 Binary Gradient Module and a Waters 2489 UV detector. Purifications were carried out on a C₁₈ Vydac 218TP1022 column (10 μM; 22×250 mm) at a flow rate of 18 mL/min. All runs used 0.1% TFA (trifluoroacetic acid) in water (solvent A) and 90% acetonitrile in water with 0.1% TFA (solvent B). Unless otherwise stated, peptides and proteins were analyzed using the following gradient: 0% B for 2 minutes (isocratic) followed by 0-73% B over 30 minutes. Electrospray ionization mass spectrometric analysis (ESI-MS) was performed on a Bruker Daltonics MicroTOF-Q II mass spectrometer. Size-exclusion chromatography was carried out on an AKTA FPLC system (GE Healthcare) using a Superdex S75 16/60 (CV=125 mL) column. Coomassie-stained gels and western blots were imaged using a LI-COR Odyssey Infrared Imager. Fluorescent gels were imaged using a GE ImageQuant LAS 4000 Imager. The splicing-dependent E. coli growth assay was performed on a VersaMax tunable microplate reader from Molecular Devices. Cell lysis was carried out using a S-450D Branson Digital Sonifier.

### Cloning of DNA Plasmids

All N-intein constructs for E. coli expression were cloned into previously used pET and pTXB1 vectors.¹ Plasmids encoding for WT pet30-His₆-SUMO-AEY-Ssp^{N}, pet30-His₆-SUMO-AEY-Npu^{N}, pTXB1-Ssp^{C}-MxeGyrA-His₆, and pTXB1-Npu^{C}-MxeGyrA-His₆ plasmids were cloned as previously described¹ and encode for the following protein sequences. Protein products after either SUMO cleavage (N-inteins) or thiolysis (C-inteins) are shown in bold for all plasmids.

### Plasmid 1:

WT Ssp^{N}: pet30-His₆-SUMO-AEY-Ssp^{N}
(SEQ ID NO: 361)
MGSSHHHHHHGSGLVPRGSASMSDSEVNQEAKPEVKPEVKPETHINLKV
SDGSSEIFFKIKKTTPLRRLMEAFAKRQGKEMDSLRFLYDGIRIQADQT
PEDLDMEDNDIIEAHREQIGGAEYCLSFGTEILTVEYGPLPIGKIVSEE
INCSVYSVDPEGRVVTQAIAQWHDRGEQEVLEYELEDGSVIRATSDHRF
LTTDYQLLAIEEIFARQLDLLTLENIKQTEEALDNHRLPFPLLDAGTIK

### Plasmid 2:

WT Npu^{N}: pet30-His₆-SUMO-AEY-Npu^{N}
(SEQ ID NO: 362)
MGSSHHHHHHGSGLVPRGSASMSDSEVNQEAKPEVKPEVKPETHINLKV
SDGSSEIFFKIKKTTPLRRLMEAFAKRQGKEMDSLRFLYDGIRIQADQT
PEDLDMEDNDIIEAHREQIGGAEYALSYETEILTVEYGLLPIGKIVEKR
IECTVYSVDNNGNIYTQPVAQWHDRGEQEVFEYCLEDGSLIRATKDHKF
MTVDGQMLPIDEIFERELDLMRVDNLPN

### Plasmid 3:

WT Ssp^{C}: pTXB1-Ssp^{C}-MxeGyrA-His₆
(SEQ ID NO: 363)
MVKVIGRRSLGVQRIFDIGLPQDHNFLLANGAIAANCITGDALVALPEG
ESVRIADIVPGARPNSDNAIDLKVLDRHGNPVLADRLFHSGEHPVYTVR
TVEGLRVTGTANHPLLCLVDVAGVPTLLWKLIDEIKPGDYAVIQRSAFS
VDCAGFARGKPEFAPTTYTVGVPGLVRFLEAHHRDPDAQAIADELTDGR
FYYAKVASVTDAGVQPVYSLRVDTADHAFITNGFVSHAHHHHHH

WT Npu^{C}: pTXB1-Npu^{C}-MxeGyrA-His₆
(SEQ ID NO: 364)
MIKIATRKYLGKQNVYDIGVERDHNFALKNGFIASNCITGDALVALPEG
ESVRIADIVPGARPNSDNAIDLKVLDRHGNPVLADRLFHSGEHPVYTVR
TVEGLRVTGTANHPLLCLVDVAGVPTLLWKLIDEIKPGDYAVIQRSAFS
VDCAGFARGKPEFAPTTYTVGVPGLVRFLEAHHRDPDAQAIADELTDGR
FYYAKVASVTDAGVQPVYSLRVDTADHAFITNGFVSHAHHHHHH

### Plasmid 4:

All Ssp^{N} batch mutants were cloned using the QuikChange site directed mutagenesis kit using plasmid 1 as a template and encode for the protein sequences shown below. The N-intein sequence is shown in bold with the residues corresponding to the batch mutation underlined.

### Plasmid 5:

Batch 1: Pet30-His₆-SUMO-AEY-Ssp^{N} (R73K, L75M,
Y79G, L81M)
(SEQ ID NO: 365)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVLEYELEDGSVIRATSDHKFMTTD
GQMLAIEEIFARQLDLLTLENIKQTEEALDNHRLPFPLLDAGTIK

### Plasmid 6:

Ssp^{N} R73K: Pet30-His₆-SUMO-AEY-Ssp^{N} (R73K)
(SEQ ID NO: 366)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVLEYELEDGSVIRATSDHKFLTTD
YQLLAIEEIFARQLDLLTLENIKQTEEALDNHRLPFPLLDAGTIK

### Plasmid 7:

Ssp^{N} R73K Y79G: Pet30-His₆-SUMO-AEY-Ssp^{N} (R73K,
Y79G)
(SEQ ID NO: 367)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVLEYELEDGSVIRATSDHKFLTTD
GQLLAIEEIFARQLDLLTLENIKQTEEALDNHRLPFPLLDAGTIK

### Plasmid 8:

Ssp^{N} R73K Y79G L81M: Pet30-His₆-SUMO-AEY-Ssp^{N}
(R73K, Y79G, L81M)
(SEQ ID NO: 368)
MGSSHHHHHHGSGLVPRGSASMSDSEVNQEAKPEVKPEVKPETHINLKVS
DGSSEIFFKIKKTTPLRRLMEAFAKQGKEMDSLRFLYDGIRIQADQTPED
LDMEDNDIIEAHREQIGGAEYCLSFGTEILTVEYGPLPIGKIVSEEINCS
VYSVDPEGRVYTQAIAQWHDRGEQEVLEYELEDGSVIRATSDHKFLTTDG
QMLAIEEIFARQLDLLTLENIKQTEEALDNHRLPFPLLDAGTIK

### Plasmid 9:

Batch 2: Pet30-His₆-SUMO-AEY-Ssp^{N} (L56F, S70K,
A83P, E85D)
(SEQ ID NO: 369)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVFEYELEDGSVIRATKDHRFLTTD
YQLLPIDEIFARQLDLLTLENIKQTEEALDNHRLPFPLLDAGTIK

### Plasmid 10:

Ssp^{N} A83P: Pet30-His₆-SUMO-AEY-Ssp^{N} (A38P)
(SEQ ID NO: 370)
MGSSHHHHHHGSGLVPRGSASMSDSEVNQEAKPEVKPEVKPETHINLKVS
DGSSEIFFKIKKTTPLRRLMEAKFAKRQGKEMDSLRFLYDGIRIQADQTP
EDLDMEDNDIIEHREQIGGAEYCLSFGTEILTVEYGPLPIGKIVSEEINC
SVYSVDPEFRVYTQAIAQWHDRGEQEVLEYELEDGSVIRATSDHRFLTTD
YQLLPIEEIFARQLDLLTLENIKQTEEALDNHRLPFPLLDAGTIK

### Plasmid 11:

Ssp^{N} S37K A83P: Pet30-His₆-SUMO-AEY-Ssp^{N} (S70K,
A83P)
(SEQ ID NO: 371)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVLEYELEDGSVIRATKDHRFLTTD
YQLLPIEEIFARQLDLLTLENIKQTEEALDNHRLPFPLLDAGTIK

### Plasmid 12:

Ssp^{N} L56, S70K, A83P: Pet30-His₆-SUMO-AEY-Ssp^{N}
(L56F, S70K, A83P)
(SEQ ID NO: 372)
MGSSHHHHHHGSGLVPRGSASMSDSEVNQEAKPEVKPEVKPETHINLKVS
DGSSEIFFKIKKTTPLRRLMEAKAKRQGKEMDSLRFLYDGIRIQADQTPE
DLDMEDNDIIEAHREQIGGAEYCLSFGTEILTVEYGPLPIGKIVSEEINC
SVYSVDPEGRVYTQAIAQWHDRGEQEVFEYELEDGSVIRATKDHRFLTTD
YQLLPIEEIFARQLDLLTLENIKQTEEALDNHRLPFPLLDAGTIK

### Plasmid 13:

Batch 3: Pet30-His₆-SUMO-AEY-Ssp^{N} (S23E, E24K,
E25R, N27E)
(SEQ ID NO: 373)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSFGTEILTVEYGPLPIGKIVEKRIEC
SVYSVDPEGRVYTQAIAQWHDRGEQEVLEYELEDGSVIRATSDHRFLTTD
YQLLAIEEIFARQLDLLTLENIKQTEEALDNHRLPFPLLDAGTIK

### Plasmid 14:

Batch 4: Pet30-His₆-SUMO-AEY-Ssp^{N} (P35N, E36N,
R38N, V39I)
(SEQ ID NO: 374)
MGSSHHHHHHGSGLVPRGSASMSDSEVNQEAKPEVKPEVKPETHINLKVS
DGSSEIFFKIKKTTPLRRLMEAKAKRQGKEMDSLRFLYDGIRIQADQTPE
DLDMEDNDIIEAHREQIGGAEYCLSFGTEILTVEYGPLPIGKIVSEEINC
SVYSVDNNGNIYTQAIAQWHDRGEQEVLEYELEDGSVIRATSDHRFLTTD
YQLLAIEEIFARQLDLLTLENIKQTEEALDNHRLPFPLLDAGTIK

The four batch mutants (Batches 5-8) and A136S point mutant on the Ssp^{C} intein were cloned by inverse PCR using Pfu Ultra II HS Polymerase (Agilent) using plasmid 3 as a template and code the protein sequences shown below:

### Plasmid 15:

Batch 5: pTXB1-Ssp^{C}-MxeGyrA-His₆ (V103I, V105I,
I106A, G107T)
(SEQ ID NO: 375)
MIKIATRRSLGVQRIFDIGLPQDHNFLLANGAIAANCITGDALVALPEGE
SVRIADIVPGARPNSDNAIDLKVLDRHGNPLVLADRLFHSGEHPVYTVRT
VEGLRVTGTANHPLLCLVDVAGVPTLLWKLIKEIKPGDYAVIQRSAFSVD
CAGFARGKPEEAPTTYTVGVPGLVRFLEAHHRDPDAQAIADELTDGRFYY
AKVASVTDAGVQPVYSLRVDTADHAFITNGFVSHAHHHHHH

### Plasmid 16:

Batch 6: pTXB1-Ssp^{C}-MxeGyrA-His₆ (R115N, I116V,
F117Y)
(SEQ ID NO: 376)
MVKVIGRRSLGVQNVYDIGLPQDHNFLLANGAIAANCITGDALVALPEGE
SVRIADIVPGARPNSDNAIDLKVLDRHGNPVLADRLFHSGEHPVYTVRTV
EGLRVTGTANHPLLCLVDVAGVPTLLWKLIKEIKPGDYAVIQRSAFSVDC
AGFARGKPEFAPTTYTVGVPGLVRFLEAHHRDPDAQAIADELTDGRFYYA
KVASVTDAGVQPVYSLRVDTADHAFITNGFVSHAHHHHHH

### Plasmid 17:

Batch 7 pTXB1-Ssp^{C}-MxeGyrA-His₆ (L121V, P122E,
Q123R)
(SEQ ID NO: 377)
MVKVIGRRSLGVQRIFDIGVERDHNFLLANGAIAANCITGDALVALPEGE
SVRIADIVPGARPNSDNAIDLKVLDRHGNPVLADRLFHSGEHPVYTVRTV
EGLRVTGTANHPLLCLVDVAGVPTLLWKLIKEIKPGDYAVIQRSAFSVDC
AGFARGKPEFAPTTYTVGVPGLVRFLEAHHRDPDAQAIADELTDGRFYYA
KVASVTDAGVQPVYSLRVDTADHAFITNGFVSHAHHHHHH

### Plasmid 18:

Batch 8: pTXB1-Ssp^{C}-MxeGyrA-His₆ (L128A, A130K,
A133F)
(SEQ ID NO: 378)
MVKVIGRRSLGVQRIFDIGLPQDHNFALKNGFIAANCITGDALVALPEGE
SVRIADIVPGARPNSDNAIDLKVLDRHGNPVLADRLFHSGEHPVYTVRTV
EGLRVTGTANHPLLCLVDVAGVPTLLWKLIKEIKPGDYAVIQRSAFSVDC
AGFARGKPEFAPTTYTVGVPGLVRFLEAHHRDPDAQAIADELTDGRFYYA
KVASVTDAGVQPVYSLRVDTADHAFITNGFVSHAHHHHHH

### Plasmid 19:

Ssp^{C} A136S: pTXB1-Ssp^{C}-MxeGyrA-His₆ (A136S)
(SEQ ID NO: 379)
MVKVIGRRSLGVQRIFDIGLPQDHNFLLANGAIASNCITGDALVALPEGE
SVRIADIVPGARPNSDNAIDLKVLDRHGNPVLADRLFHSGEHPVYTVRTV
EGLRVTGTANHPLLCLVDVAGVPTLLWKLIDEIKPGDYAVIQRSAFSVDC
AGFARGKPEFAPTTYTVGVPGLVRFLEAHHRDPDAQAIADELTDGRFYYA
KVASVTDAGVQPVYSLRVDTADHAFITNGFVSHAHHHHHH

The gene for the fused Consensus DnaE sequence was codon-optimized for E. coli expression through IDT DNA and purchased as a gBlock. The DNA gBlock sequence is shown below:

(SEQ ID NO: 380)
TGCCTGTCTTACGACACAGAGATTCTGACCGTTGAATATGGATTCCTTCC
TATCGGTAAGATCGTGGAGGAACGGATTGAATGCACAGTCTATACGGTAG
ATAAAAATGGCTTTGTGTATACACAACCTATTGCTCAGTGGCATAACCGG
GGAGAACAGGAAGTTTTCGAATACTGCTTAGAAGACGGTTCGATTATCCG
TGCAACGAAAGATCACAAATTTATGACGACCGACGGTCAGATGTTACCGA
TTGATGAGATTTTCGAACGGGGGTTAGACCTGAAACAAGTTGATGGTTTG
CCGATGGTCAAGATCATTAGTCGTAAGAGTCTGGGCACTCAAAACGTCTA
CGATATTGGAGTAGAAAAAGATCATAATTTTTTGCTGAAGAATGGGCTGG
TGGCCTCTAAC

The expression plasmid for Cfa^{N} was cloned using Gibson assembly into plasmid 1, yielding a vector coding for the following protein shown below:

### Plasmid 20:

Cfa^{N}: pET3-His₆-SUMO-AEY-Cfa^{N}
(SEQ ID NO: 381)
MGSSHHHHHHGSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGGAEYCLSYDTEILTVEYGFLPIGKIVEERIEC
TVYTVDKNGFVYTQPIAQWHNRGEQEVFEYCLEDGSIIRATKDHKFMTTD
GQMLPIDEIFERGLDLKQVDGLP

The expression plasmid for the Consensus C-intein was cloned using Gibson Assembly into plasmid 3, yielding a vector coding for the following gene:

### Plasmid 21:

Cfa^{C}: pTXB1-Cfa^{C}-MxeGyrA-H6
(SEQ ID NO: 382)
MVKIISRKSLGTQNVYDIGVEKDHNFLLKNGLVASNCITGDALVALPEGE
SVRIADIVPGARPNSDNAIDLKVLDRHGNPVLADRLFHSGEHPVYTVRTV
EGLRVTGTANHPLLCLVDVAGVPTLLVKLIDEIKPGDYAVIQRSAFSVDC
AGFARGKPEFAPTTYTVGVPGLVRFLEAHHRDPDAQAIADELTDGRFYYA
KVASVTDAGVQPVYSLRVDTADHAFITNGFVSHAHHHHHH

Cfa constructs used for E. coli growth screen.

Cfa plasmids used to screen the dependency of splicing at the +2 position of the C-extein were generating using restriction cloning into a previously generated plasmid² containing a dual expression system of the split aminoglycoside phosphotransferase (Kan^{R}) gene. The Cfa dual expression construct is shown below:

### Plasmids 22-25

[KanR promoter]-[RBS]-[KanR^{N}]-[Cfa^{N}]-[iRBS]-[Cfa^{C}-[CXN-KanR^{C}]

Following the promoter sequence, there are two separate E. coli ribosomal binding sites in this vector (RBS and iRBS). Each RBS is followed by one half of the split KanR-Intein construct, whose protein sequences are shown below (the Cfa intein is highlighted in bold).

KanR^{N}-Cfa^{N}:
(SEQ ID NO: 384)
MEQKLISEEDLSHIQRETSCSRPRLNSNMDADLYGYKWARDNVGQSGATI
YRLYGKPDAPELFLKHGKGSVANDVTDEMVRLNWLTEFMPLPTIKHFIRT
PDDAWLLTTAIPGKTAFQVLEEYPDSGENIVDALAVFLRRLHSIPVCNCP
FNSDRVFRLAQAQSRMNNGLVDASDFDDERNGWPVEQVWKEMHKLLPFCL
SYDTEILTVEYGFLPIGKIVEERIECTVYTVDKNGFVYTQPIAQWHNRGE
QEVFEYCLEDGSIIRATKDHKFMTTDGQMLPIDEIFERGLDLKQVDGLP
Cfa^{C}-KanR^{C}
(SEQ ID NO: 385)
MVKIISRKSLGTQNVYDIGVEKDHNFLLKNGLVASNCXNSVVTHGDFSLD
NLIFDEGKLIGCIDVGRVGIADRYQDLAILWNCLGEFSPSLQKRLFQKYG
IDNPDMNKLQFHLMLDEFF

The +2 position of the C-extein is underlined, and is either phenylalanine, glycine, arginine, or glutamate.

### αDEC205-HC-Cfa^{N}

pCMV Plasmids containing the αDEC205 antibody light chain (LC), heavy chain (HC), and HC-intein fusions (HC-Npu^{N}, HC-Mcht^{N}, HC-Ava^{N}) were obtained as previously described.³ A codon-optimized Cfa DnaE sequence for mammalian cell expression was generated using JCATV and purchased as a gBlock through IDT DNA. The sequence is shown below:

(SEQ ID NO: 386)
TGCCTGAGCTACGACACCGAGATCCTGACCGTGGAGTACGGCTTCCTGCC
CATCGGCAAGATCGTGGAGGAGCGCATCGAGTGCACCGTGTACACCGTGG
ACAAGAACGGCTTCGTGTACACCCAGCCCATCGCCCAGTGGCACAACCGC
GGCGAGCAGGAGGTGTTCGAGTACTGCCTGGAGGACGGCAGCATCATCCG
CGCCACCAAGGACCACAAGTTCATGACCACCGACGGCCAGATGCTGCCCA
TCGACGAGATCTTCGAGCGCGGCCTGGACCTGAAGCAGGTGGACGGCCTG
CCCGTGAAGATCATCAGCCGCAAGAGCCTGGGCACCCAGAACGTGTACGA
CATCGGCGTGGAGAAGGACCACAACTTCCTGCTGAAGAACGGCCTGGTGG
CCAGCAAC

The mammalian codon-optimized Cfa^{N} sequence was then cloned into the pCMV HC-Npu^{N} plasmid using restriction cloning to give a sequence coding for the following protein:

### Plasmid 26:

HC-Cfa^{N}: pCMV-HC-Cfa^{N}
(SEQ ID NO: 387)
MGWSCIILFLVATATGVHSEVKLLESGGGLVQPGGSLRLSCAASGFTFND
FYMNWIRQPPGQAPEWLGVIRNKGNGYTTEVNTSVKGRFTISRDNTQNIL
YLQMNSLRAEDTAIYYCARGGPYYYSGDDAPYWGQGVMVTVSSATTKGPS
VYPLAPSGSAAQTNSMVTLGCLVKGYFPEPVTVTWNSGLSLSSGVHTFPA
VLQSDLYTLSSSVTVPSSTWPSETVTCNVAHPASSTKVDKKIVPRDCGCK
PCICTVPEVSSVFIFPPKPKDVLTITLTPKVTCVVVAISKDDPEVQFSWF
VDDVEVHTAQTQPREEQFNSTFRSVSELPIMHQDWLNGKEFKCRVNSAAF
PAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVL
HEGLHNHHTEKSLSHSPGKASGGCLSYDTEILTVEYGFLPIGKIVEERIE
CTVYTVDKNGFVYTQPIAQWHNRGEQEVFEYCLEDGSIIRATKDHKFMTT
DGQMLPIDEIFERGLDLKQVDGLPGHHHHHHG

### Cfa^{C} Intein for Ligation of Dendrimer:

A plasmid containing the Cfa C-intein with a C-extein linker was cloned by inverse PCR into plasmid 21 and codes for the protein sequence shown below:

### Plasmid 27:

Cfa^{C}-link: pTXB1-H6-Cfa^{C}-CFNSGG-MxeGyrA-H6
(SEQ ID NO: 388)
MGHHHHHHSGVKIISRKSLGTQNVYDIGVEKDHNFLLKNGLVASNCFNSG
GCITGDALVALPEGESVRIADIVPGARPNSDNAIDLKVLDRHGNPVLADR
LFHSGEHPVYTVRTVEGLRVTGTANHPLLCLVDVAGVPTLLWKLIKEIKP
GDYAVIQRSAFSVDCAGFARGKPEFAPTTYTVGVPGLVRFLEAHHRDPDA
QAIADELTDGRFYYAKVASVTDAGVQPVYSLRVDTADHAFITNGFVSHAH
HHHHH

The expression and purification protocols of all His₆-SUMO-AEY-Int^{N} (plasmids 1, 2, 5-14, 20) and Int^{C}-GyrA-His₆ (plasmids 3,4, 15-19, 21 27) constructs were adapted from previously described methods.¹

Expression of all His₆-SUMO-AEY-Int^{N} Constructs

E. coli BL21(DE3) cells were transformed with an N-intein plasmid and grown at 37° C. in 1 L of LB containing 50 μg/mL of kanamycin. Once the culture had reached an OD₆₀₀=0.6, 0.5 mM IPTG was added to induce expression (0.5 mM final concentration, 3 hr at 37° C.). The cells were pelleted via centrifugation (10,500 rcf, 30 min) and stored at −80° C.

Purification of all His₆-SUMO-AEY-Int^{N} Constructs

### Purification of N-Intein Constructs for Batch Mutagenesis

The cell pellets (from expression of plasmids 1,2, 5-14) were resuspended in 30 mL of lysis buffer (50 mM phosphate, 300 mM NaCl, 5 mM imidazole, pH 8.0) containing Roche Complete protease inhibitor cocktail. The resuspended cells were then lysed by sonication on ice (35% amplitude, 8×20 second pulses on/30 seconds off). The insoluble inclusion body containing the N-intein was recovered by centrifugation (35,000 rcf, 30 min). The supernatant was discarded and the pellet was resuspended in 30 mL of Triton wash buffer (lysis buffer with 0.1% triton X-100) and incubated at room temperature for 30 minutes. The Triton wash was then centrifuged at 35,000 rcf for 30 minutes. The supernatant was discarded, the inclusion body pellet was resuspended in 30 mL of lysis buffer containing 6M Urea, and the suspension was incubated overnight at 4° C. to extract and resolubilize the protein. This mixture was then centrifuged at 35,000 rcf for 30 minutes.

The supernatant was then mixed with 4 mL of Ni-NTA resin (for affinity purification using the His₆ tag) and incubated at 4° C. for 30 minutes to batch bind the protein. This mixture was loaded on a fritted column, the flow through was collected, and the column was washed with 5 column volumes (CV) of lysis buffer with 6M Urea and 5 CV of lysis buffer with 25 mM imidazole and 6M urea. The protein was then eluted in four 1.5 CV fractions of lysis buffer with 250 mM imidazole and 6M Urea. The first two elution fractions were generally found by SDS-PAGE (12% Bis-Tris gel, run for 50 minutes at 170V) to contain the expressed protein and were combined for refolding.

The N-inteins were refolded by stepwise dialysis into lysis buffer with 0.5 mM DTT at 4° C. This refolded protein was then treated with 10 mM TCEP and Ulp1 protease (overnight, RT) to cleave the His₆-SUMO expression tag. The solution was then mixed with 4 mL Ni-NTA resin and incubated for 30 minutes at 4° C. The slurry was applied to a fritted column and the flow through was collected together with a 3 CV wash with lysis buffer. The protein was then treated with 10 mM TCEP, concentrated to 10 mL, and further purified by size exclusion chromatography using an S75 16/60 gel filtration column employing degassed splicing buffer (100 mM sodium phosphate, 150 mM NaCl, 1 mM EDTA, pH 7.2) as the mobile phase. Fractions were analyzed by SDS-PAGE, analytical RP-HPLC, and ESI-MS. Pure protein was stored by flash-freezing in liquid N₂ following the addition of glycerol (20% v/v). Note: during the refolding step, significant protein precipitation was observed for Batch 3, suggesting it is prone to aggregation.

### Purification of Cfa^{N}:

The cell pellet (from expression of plasmid 20) was first resuspended in 30 mL of lysis buffer (50 mM phosphate, 300 mM NaCl, 5 mM imidazole, pH 8.0) containing the Roche Complete protease inhibitor cocktail. The cells were then lysed by sonication (35% amplitude, 8×20 second pulses on/30 seconds off), and the lysate was pelleted by centrifugation (35,000 rcf, 30 min). The supernatant was incubated with 4 mL of Ni-NTA resin for 30 minutes at 4° C. to enrich for the soluble Cfa^{N} protein. The slurry was then loaded onto a fritted column, and the column was washed with 20 mL of wash buffer 1 (lysis buffer) followed by 20 mL of wash buffer 2 (lysis buffer with 25 mM imidazole). Finally, the protein was eluted from the column with 4×1.5 CV of elution buffer (lysis buffer+250 mM imidazole).

The desired protein, which was present in elution fractions 1 and 2 as determined by SDS-PAGE (12% bis-tris gel run in MES-SDS running buffer at 170V for 50 minutes), was then dialyzed into lysis buffer for 4 hours at 4° C. Following dialysis, the protein was treated with 10 mM TCEP and Ulp1 protease overnight at room temperature to cleave the His₆-SUMO expression tag. The solution was then incubated with 4 mL Ni-NTA resin for 30 minutes at 4° C. The slurry was applied to a fritted column and the flow through was collected together with a 3 CV wash with lysis buffer. The protein was then treated with 10 mM TCEP, concentrated to 10 mL, and purified over an S75 16/60 gel filtration column employing degassed splicing buffer (100 mM sodium phosphate, 150 mM NaCl, 1 mM EDTA, pH 7.2) as the mobile phase. Fractions were analyzed by SDS-PAGE (12% bis-tris gel run in MES-SDS running buffer at 170V for 60 minutes), analytical RP-HPLC, and ESI-MS. Pure Protein was stored in glycerol (20% v/v) and flash-frozen in liquid N₂.

### Semisynthesis of Int^{C}-CFN Constructs

E. Coli BL21 (DE3) cells were transformed with the appropriate pTXB1-Int^{C}-GyrA-H₆ plasmid (plasmids 3,4, 15-19, 21) and grown in 2 L of LB media containing ampicillin (100 μg/mL) at 37° C. Once the culture had reached an OD₆₀₀=0.6, expression was induced by the addition of IPTG (0.5 mM, 3 hours, 37° C.). Cell pellets were harvested by centrifugation (10,500 rcf, 30 min), resuspended in lysis buffer, and lysed by sonication on ice (35% amplitude, 10×20 second pulses on/30 seconds off). The protein in the soluble fraction was isolated by centrifugation (35,000 rcf, 30 mm) and then enriched by Ni-NTA purification (4 mL beads, carried out as described for N-intein constructs). Following elution in lysis buffer with 250 mM imidazole, the imidazole was removed by dialysis into fresh lysis buffer. The ligation was then carried out overnight at room temperature with the addition of 10 mM TCEP, the Roche Complete protease inhibitor cocktail, 100 mM MESNa, 5 mM EDTA, and 5 mM CFN—NH₂ (pH 7.0). The ligated Int^{C}-CFN peptide was acidified with 0.5% TFA and purified via RP-HPLC on a C₁₈ preparative column: Gradient=10% B for 10 minutes (isocratic) followed by 20-60% B over 60 minutes. The purity of each protein was determined by analytical RP-HPLC and its identity was confirmed by ESI-MS.

### Isolation of Cfa^{C}-Link-MESNa

The Cfa^{C}-link-MESNa peptide used for the semisynthesis of the Intein-dendrimer fusion was expressed and purified exactly as described above for the Int^{C}-CFN constructs (expression from plasmid 27). However, no tripeptide was added during the final ligation step, instead resulting in thiolysis of the intein and formation of an α-thioester. This Cfa^{C}-MESNa α-thioester was then purified by preparative RP-HPLC. Fractions were analyzed by ESI-MS, combined, and lyophilized.

Analysis of protein trans-splicing by RP-HPLC and ESI-MS for Batch Mutants.

Splicing reactions were carried out as adapted from a previously described protocol.¹ Briefly, N- and C-inteins (15 μM Int^{N}, 10 μM Int^{C}) were individually preincubated in splicing buffer (100 mM sodium phosphates, 150 mM NaCl, 1 mM EDTA, pH 7.2) with 2 mM TCEP for 15 minutes. All splicing reactions were carried out at 30° C. unless otherwise indicated. Splicing reactions comparing the tolerance of Npu and Cfa to chaotropic agents were carried out with the indicated concentration of either Urea or guanidine hydrochloride. Splicing was initiated by mixing equal volumes of N- and C-inteins with aliquots removed at the indicated times and quenched by the addition of 8M guanidine hydrochloride, 4% TFA (3:1 v/v). For all splicing reactions containing either Npu^{C}-CFN or Cfa^{C}-CFN, reaction progress was monitored by RP-HPLC. For all splicing reactions containing Ssp^{C}-CFN, reaction progress was monitored by ESI-MS (samples desalted with ZipTip prior to injection) due to poor chromatographic resolution of each state as seen previously.¹ Splicing for Batch 3 and for Cfa at 80° C. (15 minute preincubation) were both observed to be inefficient, reaching ~50% completion. This is likely due to aggregation (and inactivation) of the N-intein. Note, shorter preincubations of Cfa at 80° C. led to more efficient splicing.

### Kinetic Analysis of Trans-Splicing Reactions of Batch Mutants:

Kinetic analysis was carried out as previously described.¹ Briefly, five species (1-5) are separated by RP-HPLC, and peak areas are determined. For ESI-MS, peak areas are calculated for species 1-4. Each individual peak was normalized against the total area of all peaks combined and reaction progress curves were plotted (n=3). The data were then fit in ProFit to the analytical solution to the coupled differential rate equation for the three state kinetic splicing model. Because the starting material cannot be separated from the linear thioester using this assay, the three state kinetic model collapses the binding step and the first two steps of the splicing reaction into one equilibrium. Each splicing reaction was carried out in triplicate with each replicate analyzed separately. The mean and standard deviation for all values (n=3) are reported.

### Kinetic Analysis of Overall Trans-Splicing Reactions for Npu and Cfa

All splicing reactions comparing Npu and Cfa were separated by RP-HPLC with peak areas once again calculated using the manufacturer's software. For these reactions, peak areas for the starting material and branched intermediate (species 1 and 2) and product (species 3, 4, 5) were calculated. The data was then fit to the first order rate equation using the GraphPad Prism software.

$$[ P ] ( t ) = {[ P ]}_{max} \cdot ( 1 - {e}^{- k t} )$$

Where [P] is the normalized intensity of product, [P]ₘₐₓ is this value at t=∞ (the reaction plateau), and k is the rate constant (s⁻¹). The mean and standard deviation (n=3) are reported.

Generation and refinement of the DnaE Intein Multiple Sequence alignment.

Homologues of Npu DnaE were identified through a BLAST⁵ search of the NCBI⁶ (nucleotide collection) and JGI∝⁸ databases using the Npu DnaE protein sequences. This led to the identification of 105 proteins with >60% sequence identity. For N-inteins with long C-terminal tails, the proteins were truncated to 102 residues, the length of Npu. For N-inteins from the JGI database, the point of truncation was determined by the results of the BLAST program (the last residue identified in the Blast search was selected as the truncation point). Next, a multiple sequence alignment (MSA) was generated of the fused sequence (i.e. the N-intein connected to the C-intein) of all 105 inteins in Jalview (FIGS. 7A.1 to 7A.3, 7B.1 to 7B.3, 7C.1 to 7C.3, 7D.1 to 7D.3, and 7E.1 to 7E.3).⁸ To refine the MSA for inteins predicted to splice quickly, all sequences not containing K70, M75, M81, and S136 (the ‘accelerator’ residues) were removed from the alignment, leaving behind 73 inteins predicted to have fast splicing kinetics (7F.1 to 7F.3, 7G.1 to 7G.3, and 7H.1 to 7H.3). The consensus sequence of this refined alignment of fast inteins (Cfa) was calculated in Jalview by determining the amino acid that appeared most frequently at each position. A consensus residue was not identified at positions 98 and 102 due to lack of homology in the alignment, and thus the consensus sequence was truncated to 101 amino acids and position 98 was fixed to the residue found in Npu DnaE. This consensus sequence was then aligned with Npu DnaE in Jalview to calculate its percent identity. Non-identical residues were mapped onto the crystal structure of Npu DnaE (pdb=4K15) (FIG. 1).

FIGS. 7A.1 to 7A.3, 7B.1 to 7B.3, 7C.1 to 7C.3, 7D.1 to 7D.3, 7E.1 to 7E.3, 7F.1 to 7F.3, 7G.1 to 7G.3, and 7H.1 to 7H.3 show an alignment and refinement of the DnaE intein family. FIGS. 7A.1 to 7A.3, 7B.1 to 7B.3, 7C.1 to 7C.3, 7D.1 to 7D.3, and 7E.1 to 7E.3 show the multiple sequence alignment (MSA) of the 105 members of the DnaE intein family found from a BLAST search of the JGI and NCBI sequences databases. The locations of the ‘accelerator’ residues used to filter the alignment are indicated with black arrows. 7F.1 to 7F.3, 7G.1 to 7G.3, and 7H.1 to 7H.3 show MSA of the 73 DnaE inteins predicted to demonstrate fast splicing kinetics due to the presence of all four accelerator residues.

E. coli Kan^{R} screen for Cfa extein dependency.

The protein splicing coupled kanamycin resistance (Kan^{R}) assay was carried out as previously described.²,⁹ Briefly, a plasmid coding for a fragmented aminoglycoside phosphotransferase fused to a split intein (Cfa) with either F, G, R, or E present at the +2 position of the C-extein (plasmids 22-25) was transformed into DH5u competent cells and grown in starter cultures overnight (LB Broth, 100 μg/mL ampicillin, 18 hrs). These cultures were then diluted twenty-fold into a 96 well plate, and E. coli growth was measured at various concentrations of kanamycin (2.5, 10, 25, 50, 100, 250, 1000 μg/mL kanamycin with 100 μg/mL ampicillin). The cell optical density at 650 nm (OD₆₅₀) at the 24-hour end point was fit to a dose response curve with variable slope.

$${OD}_{o b s} = {OD}_{min} + \frac{( {OD}_{Max} - {OD}_{min} )}{1 + 1 {0}^{[ ( {logIC}_{50} - log [ Kan ] ) \cdot Hillslope ]}}$$

Where ODₘᵢₙ was fixed to background absorbance at 650 nm. Each assay was carried out in triplicate, fit separately, and IC₅₀ values are reported as the mean and standard deviation of IC₅₀ for these three separate measurements.

### Protein Trans Splicing of Extracted Inclusion Body

E. coli inclusion bodies containing His₆-Sumo-Cfa^{N} expression (plasmid 20) were resuspended and extracted overnight at 4° C. in lysis buffer containing 6M urea. Following centrifugation (35,000 rcf, 30 mm), the supernatant was removed and the protein enriched by Ni-NTA under denaturing conditions (as described above). However, instead of refolding the protein, trans-splicing was directly initiated by the addition of Cfa^{C}-CFN (10 μM Cfa^{C}, 2 mM TCEP, 2 mM EDTA, 2 hrs, RT). Reaction progress was monitored by SDS-PAGE.

### αDec205-HC-Int^{N} Test Expression and Splicing

Test Expression of HC-Npu^{N}, HC-Mcht^{N}, HC-Ava^{N}, HC-Cfa^{N}

Expression of all mAb constructs was carried out as previously described.³ Briefly, plasmids encoding the uDec205-LC and the uDec205-HC-Int^{N} were co-transfected into HEK293T cells and incubated for 96 hr (5% CO₂). The cells were spun down (5 minutes, 1,000 rcf), 15 μL of media for each intein fusion was mixed with 5 μL of 4× loading dye, and run on a 12% Bis-Tris gel in MES-SDS running buffer (170V for 50 minutes). The protein was then analyzed by western blot (transferred to a PVDF membrane, blotting against αMouse IgG). Expression yield was measured as the amount of HC-Int^{N} in the media as determined by densitometry. To account for varying cell growth and survival, the yield was normalized using an α-actin blot of the HEK293T cell lysate (5 s sonication, 35% amplitude, in 1× loading dye) and then represented relative to the expression of HC-Cfa^{N}. Four replicates of this test expression were carried out, and the mean was calculated with error represented as the standard deviation.

### Protein Trans-Splicing in Growth Media

Following the 96 hr expression at 37° C. of the mAB-Ava^{N} and mAB-Cfa^{N} constructs described above, the media was spun down (1,000 rcf, 5 minutes). The supernatant was then mixed with the Cfa^{C}-CFN peptide (semisynthesis of expressed plasmid 21) and incubated for 2 hours at room temperature (1 μM Cfa^{C}-CFN, 2 mM TCEP, 2 mM EDTA). The splicing reactions were analyzed by SDS-PAGE (12% Bis-Tris run in MES-SDS running buffer at 170V for 50 minutes) followed by western blot (αMouse IgG).

### Peptide and Dendrimer Synthesis

Cys-Gly-Lys(Fluorescein). This peptide was synthesized by manual addition of reagents on the Rink Amide resin according to a previously published procedure.²

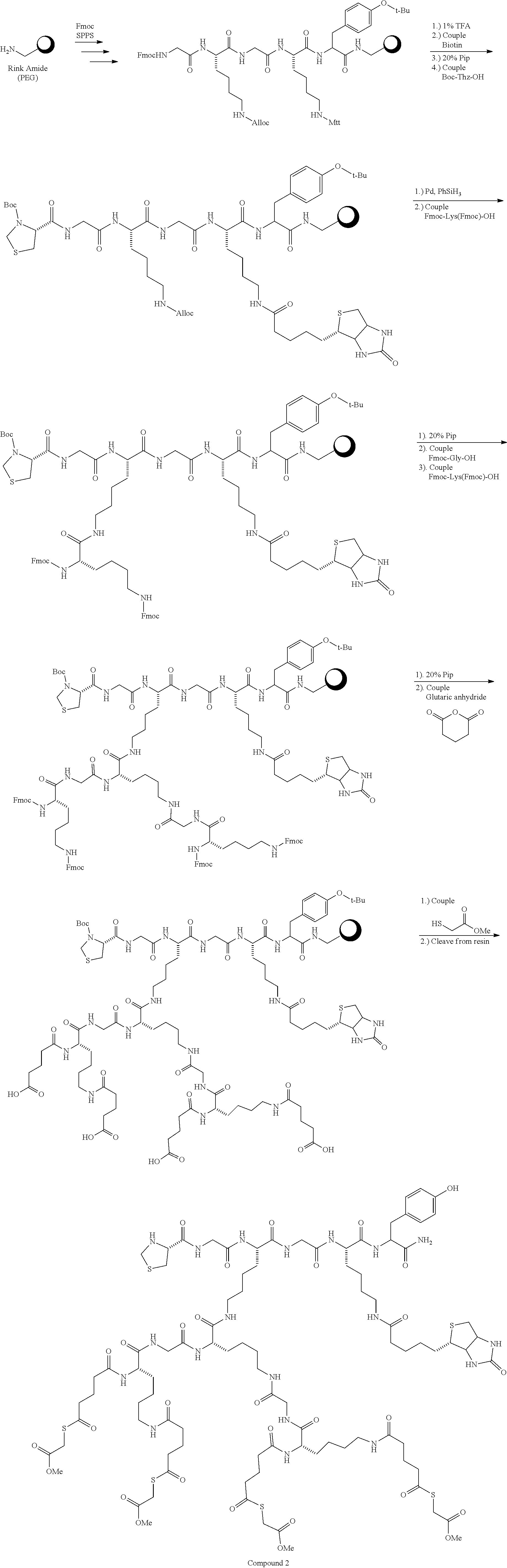

Compound 2 (dendrimer thioester). This compound was synthesized on the solid phase using the route outlined in Supplemental Scheme 1 on a scale of 400 mg of Rink Amide resin (substitution: 0.47 mmol/g, 188 μmol). General procedures are given first, followed by any specific methods for this peptide. The Fmoc group was removed with 3 mL of 20% piperidine in DMF and performed twice (one deprotection for 30 sec followed by an additional deprotection for 15 mm). After each deprotection step, as well as all subsequent synthetic steps, flow washes were used (3×5 sec. with ~5 mL of DMF each). Coupling was performed using 4 eq. of monomer, 4 eq. of either HBTU and 8 eq. of DIPEA with no pre-activation unless otherwise stated. Double couplings were used for all residues to ensure complete acylation.

The Trityl protecting group was selectively removed using 1% TFA, 5% TIS in DCM using a total of 30 mL (10×3 mL) of deprotection cocktail. Thorough washing of the resin with DCM both during and after these cycles ensured the removal of any liberated Trityl species. The resin was also neutralized with 5% DIPEA in DMF before the next coupling was undertaken. The Alloc group was deprotected using 0.1 eq of tetrakis(triphenylphosphine) palladium(0), 20 eq of phenylsilane in DCM for 3×45 min each. Thorough washing of the resin with DCM during and after these cycles was used, as well as a 5% DIPEA in DMF wash before the next coupling. The glutaric anhydride monomer was used as a preactivated dicarboxylic acid to allow the formation of the thioesters (i.e. to have a free resin-bound carboxylic acid to functionalize). 20 eq of glutaric anhydride and 10 eq of DIPEA (relative to the number of amines to be acylated) was added to the resin and allowed to react for one hour. The resin was then washed and the coupling was repeated to ensure complete reaction of the resin bound primary amines. To form the resin bound thioesters, 30 eq of methyl thioglycolate, 5 eq of PyAOP and 10 eq of DIPEA (relative to the number of carboxylates) in DMF was added to the resin and allowed to react for one hour. The resin was washed with excess DMF and the coupling procedure was repeated an additional two times.

Cleavage was performed with 95% TFA, 2.5% TIS and 2.5% H₂O for two hours at room temperature. The peptide was then precipitated with diethyl ether, dissolved in water with 0.1% TFA and analyzed via RP-HPLC. The crude material was purified via semi-preparative scale RP-HPLC, and the desired fractions were analyzed, pooled and lyophilized. RP-HPLC characterization: gradient 0-73% B, tr=18.4 min. Expected Mass: 2198.86 Da. Found: 2198.82 Da.

### Compound 3 (Dendrimer Fluorescein).

Compound 3 was synthesized by native chemical ligation (scheme 2). Compound 2 was dissolved in ligation buffer and mixed with five eq. of Cys-Gly-Lys(Fluorescein) (1 mM 2, 5 mM peptide, 4M Guanidine, 100 mM phosphate, 150 mM NaCl, 100 mM MPAA, 20 mM TCEP, pH 7.0) and allowed to react overnight at room temperature. Deprotection of the thiazolidine was then accomplished by the addition of 0.1M methoxyamine (final concentration) and decreasing the pH of the ligation buffer to 4.0 (overnight, RT).

When attempting to purify compound 3 by RP-HPLC, we noticed that it displayed poor solubility when acidified and diluted in water. However, Cys-Gly-Lys(Fluorescein), MPAA, and methoxyamine all remained in solution. From this observation, we purified 3 by selective precipitation following 10-fold dilution in water with 0.1% TFA. The precipitated powder was isolated by centrifugation (17,000 rcf, 5 min), and then redissolved (100 mM phosphate, 150 mM NaCl, pH 7.2) to wash away any remaining contaminants. Once again, the solution was precipitated by acidification and isolated by centrifugation (17,000 rcf, 5 min). This isolated powder was then lyophilized. Expected mass: 4417.8 Da. Found: 4417.5 Da.

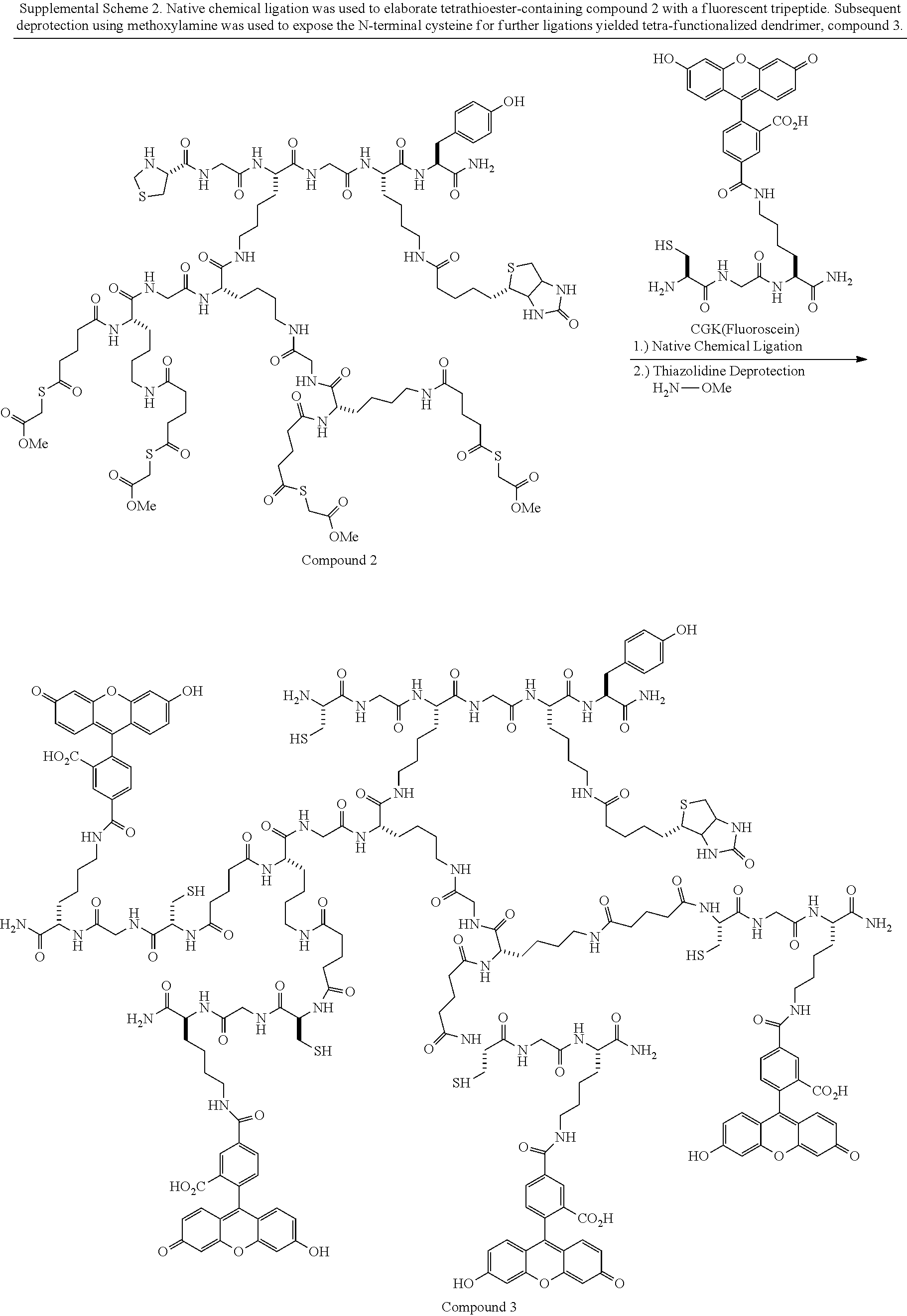

### Compound 1: (Cfa^{C}-Dendrimer)

Compound 1 was synthesized by expressed protein ligation. Compound 3 was dissolved in ligation buffer and mixed with 1.5 eq of the Cfa^{C}-MESNa thioester (100 μM 3, 150 μM Cfa^{C}-MESNa, 4M Guanidine, 100 mM phosphate, 150 mM NaCl, 20 mM TCEP, 100 mM MPAA). The reaction was allowed to proceed overnight at room temperature. The ligated product was then purified by semi-preparative RP-HPLC. Desired fractions were pooled and lyophilized. Expected mass: 9860.8 Da. Found: 9860.3 Da.

Protein Trans-Splicing of Dendrimer with αDec205 mAb.

The uDec205 mAb with Cfa^{N} fused to its C-terminus was expressed as described above. Following the 96 hr expression, the media was concentrated 10-fold in an Amicon 30K concentrator (0.5 mL). Compound 1 was dissolved in splicing buffer (100 mM phosphate, 150 mM NaCl, 1 mM EDTA, pH 7.2) and then mixed with the concentrated media (2 μM compound 1, 2 mM TCEP, 1 mM EDTA) and the reaction allowed to proceed for 2 hrs at room temperature. The splicing mixture was then analyzed by SDS-PAGE (12% Bis-Tris run in MES-SDS running buffer at 170V for 50 minutes) and imaged on a fluorescence imager. This was followed by transfer to a PVDF membrane and western blot analysis (αMouse IgG).

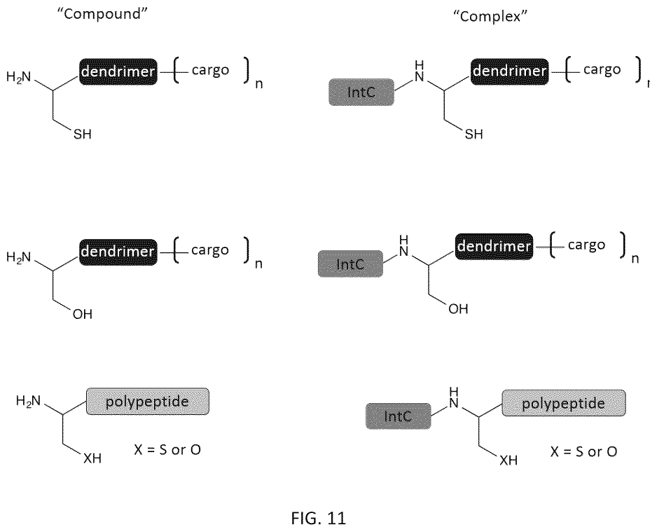

The invention allows for the formation of various complexes between a split intein fragment and a compound. Several such complexes and compounds are illustrated in the table of FIG. 11. IntC is a split intein fragment, for example, a split intein C-fragment. For example, the dendrimer can have the form of Compound 2, Compound 3, or portions of these. For example, the cargo can be a dye (e.g., fluorescein), another marker molecule, a drug (e.g., a cytotoxic molecule, such as used in the treatment of cancer), or a nucleotide. For example, the polypeptide can be a wholly or partially synthetic or a naturally occurring polypeptide or portion thereof. A dendrimer can be a molecule having a branched chemical structure onto which one or more “cargo” molecules can be “loaded”. A “cargo” molecule can be a synthetic of naturally occurring molecule. The cargo molecule can be structured to have no free 1,2-amino thiols or 1,2-amino alcohols. When the intein is bonded through an amino thiol or amino alcohol to a polypeptide, as shown in row 3 of the table of FIG. 11, the complex formed can be considered to be a recombinant fusion protein.

### Example 2

A major caveat to splicing-based methods is that all characterized inteins exhibit a sequence preference at extein residues adjacent to the splice site. In addition to a mandatory catalytic Cys, Ser, or Thr residue at position +1 (i.e., the first residue within the C-extein), there is a bias for residues resembling the proximal N- and C-extein sequence found in the native insertion site. Deviation from this preferred sequence context leads to a marked reduction in splicing activity, limiting the applicability of PTS-based methods.^{23,24} Accordingly, there is a need for split inteins whose activities are minimally affected by local sequence environment. For DnaE inteins, extein sequence preferences are largely confined to the catalytic cysteine at the +1 position and large hydrophobic residues that are preferred at the +2 position.²⁵

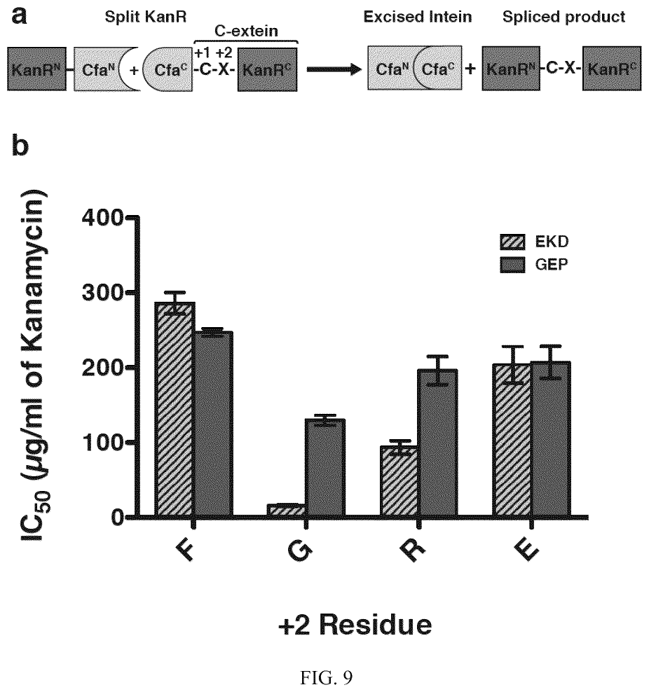

In this example, a “EKD” to “GEP” loop mutation into residues 122-124 of Cfa (Cfa_{GEP}) was engineered and resulted in increased promiscuity at the +2 position of the C-extein in a kanamycin resistance assay (FIG. 9). The EKD→GEP mutation increases the activity of Cfa under a wide range of extein contexts. In addition, it can be reasonably expected that these same (or similar) mutations will increase promiscuity among other members of the DnaE intein family (including Npu and those listed in FIGS. 7A.1 to 7A.3, 7B.1 to 7B.3, 7C.1 to 7C.3, 7D.1 to 7D.3, 7E.1 to 7E.3, 7F.1 to 7F.3, 7G.1 to 7G.3, and 7H.1 to 7H.3).

The following sequences represent the engineered inteins:

The Cfa C-intein with the “GEP” mutation that imparts more “promiscuous” activity according to an embodiment of the invention is:

(SEQ ID NO: 389)
VKIISRKSLGTQNVYDIGVGEPHNFLLKNGLVASN.

An example of a fusion intein of the Cfa N-intein and Cfa C-intein with the “GEP” mutation of SEQ ID: 389) is:

(SEQ ID NO: 390)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLPVKIISRKSLGTQNVYDIGVGEPHNFLLKNGLVASN.

FIG. 9 shows a schematic and a table showing the increased promiscuity of Cfa_{GEP}. Panel A shows a schematic depicting the PTS-dependent E. coli selection system with the Cfa split intein. The kanamycin resistance protein, KanR, is split and fused to N- and C-intein fragments (Cfa^{N} and Cfa^{C}). The +2 C-extein residue (red X) is varied in the system. In Panel B, IC₅₀ values for kanamycin resistance of the Cfa_{E}xD (WT) and Cfa_{GEP} (GEP) inteins with indicated+2 C-extein residue are shown (error=standard error (n=3)).

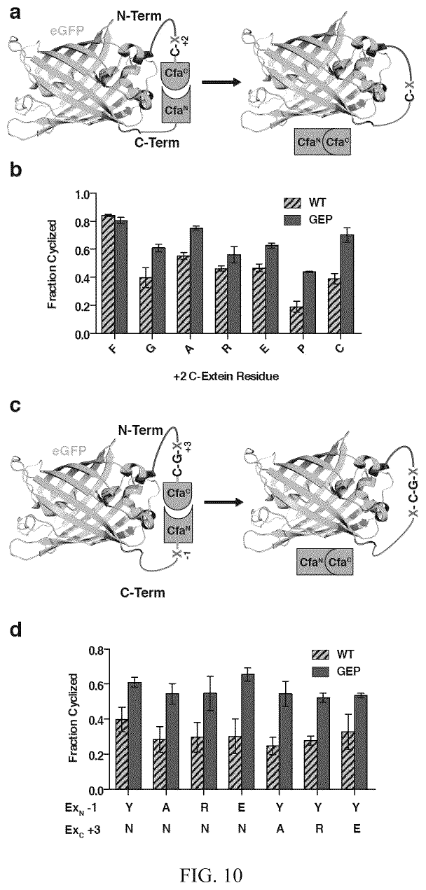

Furthermore, this same tolerance for varying extein sequences was also observed in the cyclization of eGFP in E. coli (FIG. 10). The Cfa_{GEP} intein demonstrated improved yields of cyclized product in all unfavorable+2 C-extein contexts tested (FIG. 10 panel A, FIG. 10 panel B). In addition, Cfa_{GEP} maintains this improved cyclization activity even when the −1 and +3 extein positions are varied (FIG. 10 panel C, FIG. 10 panel D). This engineered “GEP” loop sequence, which has not been identified in a wild type naturally split DnaE intein, should thus expand the breadth of proteins and peptides accessible to PTS-based technologies.

FIG. 10 shows schematics and graphs showing eGFP Cyclization with the CfaG_{EP} split intein. Panel A is a schematic depicting cyclization of eGFP in E. coli with variable residues at the +2 C-extein position (red X). In panel B, the fraction of cyclized eGFP formed after overnight expression in E. coli for Cfa_{ED} (WT) and Cfa_{GEP} (GEP) with the indicated+2 C-extein residue is shown (mean±standard deviation, n=3). Panel C is a schematic depicting the cyclization of eGFP in E. coli with variable residues at the +3 C-extein position (blue X) and −1 N-extein position (red X). Panel D shows a fraction of cyclized eGFP formed after overnight expression in E. coli for CfaEKD (WT) and Cfa_{GEP} (GEP) with the indicated+3 C-extein and −1 N-extein residues (mean±standard deviation, n=3).

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### REFERENCES

(1) Shah, N. H.; Muir, T. W. Chem. Sci. 2014, 5, 15.
(2) Wu, H.; Hu, Z.; Liu, X. Q. Proc. Natl. Acad. Sci. U.S.A 1998, 95, 9226.
(3) Iwai, H.; Zuger, S.; Jin, J.; Tam, P. H. FEBS Lett. 2006, 580, 1853.
(4) Zettler, J.; Schutz, V.; Mootz, H. D. FEBS Lett. 2009, 583, 909.
(5) Shah, N. H.; Eryilmaz, E.; Cowburn, D.; Muir, T. W. J. Am. Chem. Soc. 2013, 135, 5839.
(6) Shah, N. H.; Dann, G. P.; Vila-Perello, M.; Liu, Z.; Muir, T. W. J. Am. Chem. Soc. 2012, 134, 11338.
(7) Carvajal-Vallejos, P.; Pallisse, R.; Mootz, H. D.; Schmidt, S. R. J. Biol. Chem 2012, 287, 28686.
(8) Wu, Q.; Gao, Z.; Wei, Y.; Ma, G.; Zheng, Y.; Dong, Y.; Liu, Y. Biochem. J. 2014, 461, 247.
(9) Aranko, A. S.; Oeemig, J. S.; Kajander, T.; Iwai, H. Nat. Chem. Biol. 2013, 9, 616.
(10) Pietrokovski, S. Protein Sci. 1994, 3, 2340.
(11) Dearden, A. K.; Callahan, B.; Roey, P. V.; Li, Z.; Kumar, U.; Belfort, M.; Nayak, S. K. Protein Sci. 2013, 22, 557.
(12) Du, Z.; Shemella, P. T.; Liu, Y.; McCallum, S. A.; Pereira, B.; Nayak, S. K.; Belfort, G.; Belfort, M.; Wang, C. J. Am. Chem. Soc. 2009, 131, 11581.
(13) Lehmann, M.; Kostrewa, D.; Wyss, M.; Brugger, R.; D'Arcy, A.; Pasamontes, L.; van Loon, A. P. Protein Eng. 2000, 13, 49.
(14) Steipe, B. Methods Enzymol. 2004, 388, 176.
(15) Altschul, S. F.; Gish, W.; Miller, W.; Myers, E. W.; Lipman, D. J. J. Mol. Biol. 1990, 215, 403.
(16) Grigoriev, I. V.; Nordberg, H.; Shabalov, I.; Aerts, A.; Cantor, M.; Goodstein, D.; Kuo, A.; Minovitsky, S.; Nikitin, R.; Ohm, R. A.; Otillar, R.; Poliakov, A.; Ratnere, I.; Riley, R.; Smirnova, T.; Rokhsar, D.; Dubchak, I. Nucleic Acids Res. 2012, 40, D26.
(17) Tatusova, T.; Ciufo, S.; Fedorov, B.; O'Neill, K.; Tolstoy, I. Nucleic Acids Res. 2014, 42, D553.
(18) Shah, N. H.; Eryilmaz, E.; Cowburn, D.; Muir, T. W. J. Am. Chem. Soc. 2013, 135, 18673.
(19) Mohlmann, S.; Bringmann, P.; Greven, S.; Harrenga, A. BMC Biotechnol. 2011, 11, 76.
(20) Barbuto, S.; Idoyaga, J.; Vila-Perello, M.; Longhi, M. P.; Breton, G.; Steinman, R. M.; Muir, T. W. Nat. Chem. Biol. 2013, 9, 250.
(21) Vila-Perello, M.; Liu, Z.; Shah, N. H.; Willis, J. A.; Idoyaga, J.; Muir, T. W. J. Am. Chem. Soc. 2013, 135, 286.
(22) Shah, N. D.; Parekh, H. S.; Steptoe, R. J. Pharm. Res. 2014, 31, 3150.
(23) Iwai, H.; Zuger, S.; Jin, J.; Tam, P. H. FEBS Lett. 2006, 580, 1853.
(24) Amitai, G.; Callahan, B. P.; Stanger, M. J.; Belfort, G.; Belfort, M. Proc Natl Acad Sci USA 2009, 106, 11005.
(25) Cheriyan, M.; Pedamallu, C. S.; Tori, K.; Perler, F. J Biol Chem 2013, 288, 6202.

1-24. (canceled)

25. A split intein N-fragment comprising an amino acid sequence having from 90% to less than 95% sequence identity to
(SEQ ID NO: 1)
CLSYDTEILTVEYGFLPIGKIVEERIECTVYTVDKNGFVYTQPIAQWHN
RGEQEVFEYCLEDGSIIRATKDHKFMTTDGQMLPIDEIFERGL
or to
(SEQ ID NO: 2)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP.

26. A complex comprising the split intein N-fragment of claim 25 and a compound.

27. The complex of claim 26, wherein the compound is selected from the group consisting of:
(i) a peptide or a polypeptide,
(ii) an antibody chain,
(iii) an antibody heavy chain, and
(iv) a compound comprising a peptide, an oligonucleotide, a drug or a cytotoxic molecule.

28. A split intein C-fragment comprising an amino acid sequence having from 90% to less than 95% sequence identity to VKIISRKSLGTQNVYDIGVEKDHNFLLKNGLVASN (SEQ ID NO: 3), to MVKIISRKSLGTQNVYDIGVEKDHNFLLKNGLVASN (SEQ ID NO: 4) or to VKIISRKSLGTQNVYDIGVGEPHNFLLKNGLVASN (SEQ ID NO: 389).

29. A complex comprising the split intein C-fragment of claim 28 and a compound.

30. The complex of claim 29, wherein the compound is selected from the group consisting of:
(i) a peptide or a polypeptide,
(ii) a compound comprising a peptide, an oligonucleotide, a drug, or a cytotoxic molecule,
(iii) a 1,2-amino thiol bonded to a peptide, an oligonucleotide, a drug, or a cytotoxic molecule,
(iv) a 1,2-amino alcohol bonded to a peptide, an oligonucleotide, a drug, or a cytotoxic molecule and
(v) a dendrimer.

31. The complex of claim 29, wherein the compound is a dendrimer having the structure 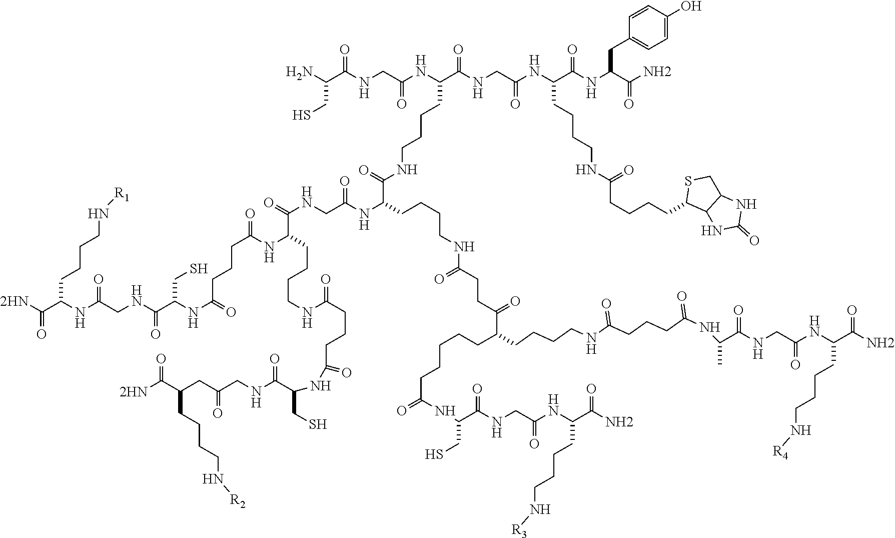
wherein R1, R2, R3, and R4 are independently selected from the group consisting of hydrogen (H) and cargo molecules.

32. The complex of claim 29, wherein R1, R2, R3, and R4 are each a dye molecule or wherein R1, R2, R3, and R4 are each a fluorescein derivative having the structure

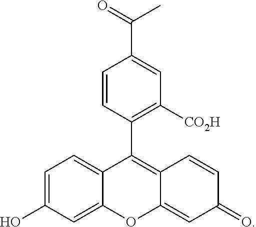 [SMILES: CC(=O)c1ccc(-c2c3ccc(=O)cc-3oc3cc(O)ccc23)c(C(=O)O)c1]

33. A complex selected from the group consisting of:
(i) a complex of the structure 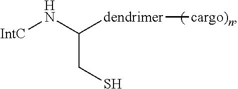
wherein IntC is the split intein C-fragment of claim 29 and
wherein n is from 0 to 8;
(ii) a complex of the structure 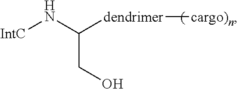
wherein IntC is the split intein C-fragment of claim 28 and
wherein n is from 0 to 8; and
(iii) a complex of the structure 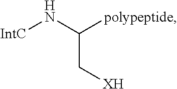
wherein IntC is the split intein C-fragment of claim 28 and
wherein X is sulfur (S) or oxygen (O).

34. A composition comprising:
a split intein N-fragment comprising an amino acid sequence having from 90% to less than 95% sequence identity to SEQ ID NO: 1 or SEQ ID NO: 2; and
the split intein C-fragment of claim 28.

35. A nucleotide plasmid comprising a nucleotide sequence encoding the split intein N-fragment of claim 25.

36. A nucleotide plasmid comprising a nucleotide sequence encoding the split intein C-fragment of claim 28.

37. A method for splicing two complexes comprising:
contacting:
a first complex comprising a first compound and a split intein N-fragment comprising an amino acid sequence having from 90% to less than 95% sequence identity to SEQ ID NO: 1 or SEQ ID NO: 2; and
a second complex comprising a second compound and the split intein C-fragment of claim 28,
wherein the contacting is performed under conditions that permit binding of the split intein N-fragment to the split intein C-fragment to form an intein intermediate; and
reacting the intein intermediate to form a conjugate of the first compound with the second compound.

38. A method comprising:
contacting:
a first complex comprising a first compound and a split intein N-fragment comprising an amino acid sequence having from 90% to less than 95% sequence identity to SEQ ID NO: 1 or SEQ ID NO: 2; and
a second complex comprising a second compound and the split intein C-fragment of claim 28,
wherein the contacting is performed under conditions that permit binding of the split intein N-fragment to the split intein C-fragment to form an intein intermediate; and
reacting the intein intermediate with a nucleophile to form a conjugate of the first compound with the nucleophile.

39. A method comprising:
fusing:
a first nucleotide sequence encoding an amino acid sequence of a split intein N-fragment comprising an amino acid sequence having from 90% to less than 95% sequence identity to SEQ ID NO: 1 or SEQ ID NO: 2; with
a second nucleotide sequence encoding an amino acid sequence of the split intein C-fragment of claim 28,
so that the fusion of the first nucleotide sequence and the second nucleotide sequence encodes for a contiguous intein.

40. The method of claim 37, wherein the first compound is a polypeptide or an antibody and/or wherein the second compound is a dendrimer or a polypeptide.

41. A kit for splicing two complexes together comprising:
a split intein N-fragment comprising an amino acid sequence having from 90% to less than 95% sequence identity to SEQ ID NO: 1 or SEQ ID NO: 2;
the split intein C-fragment of claim 28;
a reagent for binding the split intein N-fragment to the split intein C-fragment to form an intein intermediate; and
a nucleophilic agent.

42. A gene fusion comprising:
a first nucleotide sequence encoding an amino acid sequence of a split intein N-fragment comprising an amino acid sequence having from 90% to less than % sequence identity to SEQ ID NO: 1 or SEQ ID NO: 2,
fused with a second nucleotide sequence encoding an amino acid sequence of the split intein C-fragment of claim 28.

43. A polynucleotide encoding the split intein N-fragment of claim 25.

44. A polynucleotide encoding the split intein C-fragment of claim 28.


SEQUENCE LISTING (499 sequences)

SEQ ID NO: 1 - protein, 92 aa, synthetic construct
  source 1..92: note = synthetic polypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGL

SEQ ID NO: 2 - protein, 101 aa, synthetic construct
  source 1..101: note = synthetic polypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 3 - protein, 35 aa, synthetic construct
  source 1..35: note = synthetic polypeptide
VKIISRKSLGTQNVYDIGVEKDHNFLLKNGLVASN

SEQ ID NO: 4 - protein, 36 aa, synthetic construct
  source 1..36: note = synthetic polypeptide
MVKIISRKSLGTQNVYDIGVEKDHNFLLKNGLVASN

SEQ ID NO: 5 - protein, 102 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 6 - protein, 35 aa, Nostoc punctiforme
IKIATRKYLGKQNVYDIGVERDHNFALKNGFIASN

SEQ ID NO: 7 - protein, 102 aa, Chroococcidiopsis thermalis
CLSYDTEILTVEYGAIPIGKIVEERIECTVYSVDNNGFIYTQPIAQWHNRGQQEVFEYCLEDGSIIRATKDHKFMTFEGKMLPIDEIFEQELDLKQVKSIQN

SEQ ID NO: 8 - protein, 35 aa, Chroococcidiopsis thermalis
VKIISRKSLGIQPVYDIGVERDHKFVLKNGLVASN

SEQ ID NO: 9 - protein, 102 aa, Nodularia spumigena
CLSYDTEILTVEYGYIPIGEIVEKAIECSVYSVDNNGNVYTQPIAQWHNRGEQEVFEYSLEDGSTIRATKDHKFMTTDGQMLPIDEIFAQELDLLQVHGLPK

SEQ ID NO: 10 - protein, 35 aa, Nodularia spumigena
VKITARKFVGRENVYDIGVERYHNFAIKNGLIASN

SEQ ID NO: 11 - protein, 102 aa, Anabaena cylindrica
CLSYDTEVLTVEYGFIPIGEIVEKRIECSIFSVDKNGNVYTQPIAQWHNRGRQEIYEYCLDDGSKIRATKDHKFMTTAGEMLPIDEIFERDLDLLKVEGLPE

SEQ ID NO: 12 - protein, 35 aa, Anabaena cylindrica
VKIISRQYLGQADVYDIGVEEDHNFAIKNGFIASN

SEQ ID NO: 13 - protein, 102 aa, Calothrix sp.
CLSYDTEVLTVEYGLLPIGEIVEKGIECRVFSVDNHGNVYTQPIAQWHNRGQQEVFEYGLDDGSVIRATKDHKFMTTDGKMLPIDEIFERGLDLLQVQGLPE

SEQ ID NO: 14 - protein, 35 aa, Calothrix sp.
VKVITRKYIGKENVYDIGVELDHNFAIRNGLVASN

SEQ ID NO: 15 - protein, 102 aa, Nostoc sp.
CLSYDTEILTVEYGFLPIGEIVEKGIECTVFSVASNGIVYTQPIAQWHNRGQQEIFEYCLEDGSIIRATKDHKFMTQDGQMLPIDEIFACELDLLQVQGLPE

SEQ ID NO: 16 - protein, 35 aa, Nostoc sp.
VKVVTRKYIGKENVYDIGVERDHNFVIRNGLVASN

SEQ ID NO: 17 - protein, 102 aa, Nostoc azollae
CLSYKTEVLTVEYGLIPIGEIVEKRIECSLFSVDENGNIYTQPIAQWHHRGVQEVYEYCLDDGTIIRATKDHKFMTTIGEMLPIDEIFERDLNLLQVNGLPT

SEQ ID NO: 18 - protein, 35 aa, Nostoc azollae
VKIISRQFLGPANVYDIGVAQDHNFAIKNGLIASN

SEQ ID NO: 19 - protein, 102 aa, Nostoc sp.
CLSYDTEVLTVEYGFVPIGEIVEKGIECSVFSINNNGIVYTQPIAQWHHRGKQEVFEYCLEDGSIIKATKDHKFMTQDGKMLPIDEIFEQELDLLQVKGLPE

SEQ ID NO: 20 - protein, 35 aa, Nostoc sp.
IKIASRKFLGVENVYDIGVRRDHNFFIKNGLIASN

SEQ ID NO: 21 - protein, 102 aa, Anabaena variabilis
CLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPE

SEQ ID NO: 22 - protein, 35 aa, Anabaena variabilis
IKIASRKFLGVENVYDIGVGRDHNFFVKNGLIASN

SEQ ID NO: 23 - protein, 100 aa, Pleurocapsa sp.
CLSYDTKILTVEYGAMPIGKIVEEQIDCTVYTVNQNGFVYTQPIAQWHDRGKQEIFEYCLEDGSIIRATKDHKFMTTDGQMLPIDKIFEKGLDLKTINCD

SEQ ID NO: 24 - protein, 35 aa, Pleurocapsa sp.
VKILSRKSLGIQSVYDIGVEKDHNFLLANGLVASN

SEQ ID NO: 25 - protein, 100 aa, Cyanothece sp.
CLSYETQIMTVEYGLMPIGKIVEEQIDCTVYTVNKNGFVYTQPIAQWHYRGEQEVFEYCLEDGSTIRATKDHKFMTTDGQMLPIDEIFEQGLELKQIHLS

SEQ ID NO: 26 - protein, 35 aa, Cyanothece sp.
VKIISRQSLGIQPVYDIGVEKDHNFLISDGLIASN

SEQ ID NO: 27 - protein, 99 aa, Cyanothece sp.
CLSYDTEILTVEYGPMPIGKIVEEQIECTVYTVDKNGLVYTQPIAQWHHRGQQEVFEYCLEDGSIIRATKDHKFMTDDGQMLPIEEIFEKGLELKQIIL

SEQ ID NO: 28 - protein, 35 aa, Cyanothece sp.
VKIISRQLAGNQTVYDLGVEKDHNFLLANGLIASN

SEQ ID NO: 29 - protein, 102 aa, Nostoc sp.
CLSYDTQVLTVEYGLVPIGEIVEKQLECSVFTIDGHGYVYTQAIAQWHNRGQQEVFEYGLEDGSVIRATKDHKFMTTDGQMLPIDEIFERELDLLQVQGLRW

SEQ ID NO: 30 - protein, 35 aa, Nostoc sp.
VKIITRKYIGQANVYDIGVAQDHNFVIENRLIASN

SEQ ID NO: 31 - protein, 101 aa, Tolypothrix bouteillei
CLSYDTEILTVEYGFLPIGKIVEKGIECNVYSVDKNGNIYTQPIAQWHDRGEQEVFEYCLENGSVIRATKDHKFMTTSGEMLPIDEIFERGLDLIRVEDLP

SEQ ID NO: 32 - protein, 35 aa, Tolypothrix bouteillei
VKILTRKSIGKQTVYDIGVERDHNFVIKNGSVASN

SEQ ID NO: 33 - protein, 101 aa, Aphanizomenon ovalisporum
  VAR_SEQ 87: note = X can be any amino acid
CLSADTEILTVEYGFLPIGEIVGKAIECRVYSVDGNGNIYTQSIAQWHNRGEQEVFEYTLEDGSIIRATKDHKFMTTDGEMLPIDEXFARQLDLMQVQGLH

SEQ ID NO: 34 - protein, 35 aa, Aphanizomenon ovalisporum
VKITARKFVGRENVYDIGVEHHHNFAIKNGLIASN

SEQ ID NO: 35 - protein, 102 aa, Oscillatoria nigro-viridis
CLSYDTKILTVEYGPMAIGKIVEEKIECTVYSVDSNGYIYTQSIAQWHRRGQQEVFEYCLEDGSIIRATKDHKFMTVGGQMLPIDEIFEQGLDLKQINSSSD

SEQ ID NO: 36 - protein, 35 aa, Oscillatoria nigro-viridis
VKIISRKSLGTQEVYDIGVEREHNFILENSLVASN

SEQ ID NO: 37 - protein, 100 aa, Rivularia sp.
CLSYDTEVLTEEFGLIPIGKIVEEKIDCTVYSVDVNGNVYSQPIAQWHNRGMQEVFEYELEDGSTIRATKDHKFMTVDGEMLAIDEIFEKGLELKRVGIY

SEQ ID NO: 38 - protein, 35 aa, Rivularia sp.
VKIISRKVLKTENVYDIGLEGDHNFIIKDGLIASN

SEQ ID NO: 39 - protein, 102 aa, Trichodesmium erythraeum
CLTYETEIMTVEYGPLPIGKIVEYRIECTVYTVDKNGYIYTQPIAQWHNRGMQEVYEYSLEDGTVIRATPEHKFMTEDGQMLPIDEIFERNLDLKCLGTLEL

SEQ ID NO: 40 - protein, 35 aa, Trichodesmium erythraeum
VKIVSRKLAKTENVYDIGVTKDHNFVLANGLIASN

SEQ ID NO: 41 - protein, 102 aa, Microcoleus sp.
CLSYDSEILTVEYGLMPIGKIVEEGIECTVYSVDSHGYLYTQPIAQWHHRGQQEVFEYDLEDGSVIRATKDHKFMTSEGQMLAIDEIFERGLELKQVKRSQP

SEQ ID NO: 42 - protein, 35 aa, Microcoleus sp.
VKIVRRKSLGIQTVYDIGVERDHNFLLANGLVASN

SEQ ID NO: 43 - protein, 102 aa, Stanieria cyanosphaera
CLSYDTEILTVEYGAMPIGKIVKEQIECNVYTVNQNGFIYPQAIAQWHERGKQEIFEYTLDNGLVIRATKDHKFMTIDGQMLPIDEIFERGLELQRINDYSN

SEQ ID NO: 44 - protein, 35 aa, Stanieria cyanosphaera
VKIVSRKSLGKQPVYDIGVTKDHNFLLSNGVVASN

SEQ ID NO: 45 - protein, 102 aa, Calothrix sp.
CLSYDTEILTWEYGFLKIGEIVEKQILCSVFSVDEQGNVYTQPIAQWHNRGLQELFAYQLEDGGVIRATKDHKFMTTDGQMLAIDEIFERQLDLFQVKGLPE

SEQ ID NO: 46 - protein, 35 aa, Calothrix sp.; identical to SEQ ID NO: 38

SEQ ID NO: 47 - protein, 99 aa, Cyanobacterium stanieri
CLSYDTEVLTVEYGVLPIGKIVEEQIQCTVYSVDQYGFVYTQAIAQWHDRGEQEVFEYELENGATIKATKDHKMMTSDGQMLPIDQIFEQGLDLFMVSF

SEQ ID NO: 48 - protein, 35 aa, Cyanobacterium stanieri
VKIVKRRSHGIQKVYDIGVAKDHNFLLHNGLVASN

SEQ ID NO: 49 - protein, 99 aa, Cyanothece sp.
CLSYDTEILTVEYGPMPIGKIVEENINCTVYTVDPNGFVYTQAIAQWHYRGEQEIFEYYLEDGATIRATKDHKFMTMEGKMLPIDEIFENNLDLKQLTL

SEQ ID NO: 50 - protein, 35 aa, Cyanothece sp.
VKIIGRQSLGVQKVYDIGVEKEHNFLLHNGLIASN

SEQ ID NO: 51 - protein, 99 aa, Cyanothece sp.
CLSYDTEILTVEYGAIPIGKVVEENIDCTVYTVDKNGFVYTQNIAQWHLRGQQEVFEYYLDDGSILRATKDHQFMTLEGEMLPIHEIFERGLELKKIKI

SEQ ID NO: 52 - protein, 35 aa, Cyanothece sp.
VKIVSYRSLGKQFVYDIGVAQDHNFLLANGSIASN

SEQ ID NO: 53 - protein, 101 aa, Anabaena sp.
CLSYDTEILTVEYGFLEIGEIVEKQIECKVYTIDSNGMLYTQSIAQWHNRGQQEVYEYLLENGAIIRATKDHKFMTEAGQMLPIDEIFAQGLDLLQVGVAE

SEQ ID NO: 54 - protein, 35 aa, Anabaena sp.
VKIVSRTYVGQANVYDIGVESDHNFVIKNGFIASN

SEQ ID NO: 55 - protein, 102 aa, Aphanothece halophytica
CLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEM

SEQ ID NO: 56 - protein, 35 aa, Aphanothece halophytica
VKIIKRQSLGRQNVYDVCVETDHNFVLANGCVASN

SEQ ID NO: 57 - protein, 102 aa, Halothece sp.; identical to SEQ ID NO: 55

SEQ ID NO: 58 - protein, 35 aa, Halothece sp.
VKIIKRQSLGRQNVYDIGVETDHNFVLANGCVASN

SEQ ID NO: 59 - protein, 102 aa, Cyanobacterium aponinum
CLSYDTEILTVEYGAISIGKIVEEKINCQVYSVDKNGFIYTQNIAQWHDRGSQELFEYELEDGRIIKATKDHKMMTKDGQMLAINDIFEQELELYSVDDMGV

SEQ ID NO: 60 - protein, 35 aa, Cyanobacterium aponinum
VKIVKRRSLGVQPVYDIGVEKDHNFILANGLVASN

SEQ ID NO: 61 - protein, 98 aa, unidentified
  source 1..98: note = Candidatus Atelocyanobacterium thalassa isolate sequence
CLSYDTKVLTVEYGPLPIGKVVQENIRCRVYTTNDQGLIYTQPIAQWHNRGKQEIFEYHLDDKTIIRATKEHQFMTVDHVMMPIDEIFEQGLELKKIK

SEQ ID NO: 62 - protein, 35 aa, unidentified
  source 1..35: note = Candidatus Atelocyanobacterium thalassa isolate sequence
LKIIRRKSLGMHEVFDIGLEKDHNFVLSNGLIASN

SEQ ID NO: 63 - protein, 102 aa, Oscillatoria limnetica
CLSYNTEVLTVEYGPLPIGKIVDEQIHCRVYSVDENGFVYTQAIAQWHDRGYQEIFAYELADGSVIRATKDHQFMTEDGQMFPIDEIWEKGLDLKKLPTVQD

SEQ ID NO: 64 - protein, 35 aa, Oscillatoria limnetica
VKIVRRQSLGVQNVYDIGVEKDHNFLLASGEIASN

SEQ ID NO: 65 - protein, 102 aa, unidentified
  source 1..102: note = Cyanobacterium endosymbiont of Epithemia turgida sequence
CLSYDTEVLTVEYGAIPIGRMVEESLDCTVYTVDKNGFVYTQSIQQWHSRGQQEIFEYCFEDGSIIRATKDHKFMTAEGKMSSIHDIFEQGLELKKIIPWSG

SEQ ID NO: 66 - protein, 35 aa, unidentified
  source 1..35: note = Cyanobacterium endosymbiont of Epithemia turgida sequence
AKIISCKSLGKQSVYDIGVVQDHNFLLANGVVASN

SEQ ID NO: 67 - protein, 98 aa, Synechococcus sp.
CLGYDTPVLTVEYGFMPIGKIVEEKIQCHVYSVDQNGLVFTQAIAQWHNRGQQEVWEYNLDNGDIVRATKDHKFMTIDGQMLPINQIFEQGLELKVIA

SEQ ID NO: 68 - protein, 35 aa, Synechococcus sp.
VKIVSCKPLRVQTVYDIGVEKDHNFILDNGLVASN

SEQ ID NO: 69 - protein, 99 aa, Dactylococcopsis salina
CLSYDTEVLTEEYGAIPIGKIVEERMNCHVYSVDENGFIYSQPIAQWHPRGEQEVVEYTLEDGKIIRATADHKMMTETGEMLPIEQIFQQQLDLKISNQ

SEQ ID NO: 70 - protein, 35 aa, Dactylococcopsis salina
VKIINRQSLGKQTVYDIGVEKDHNFILGNGLVASN

SEQ ID NO: 71 - protein, 102 aa, Cylindrospermum stagnale
CLSYDTEILTVEYGFIPIGEIVEKRIECSVYSVDNHGNVYTQPIAQWHNRGLQEVFEYCLEDGSTIRATKDHKFMTTDKEMLPIDEIFERGLDLLRVEGLPI

SEQ ID NO: 72 - protein, 35 aa, Cylindrospermum stagnale
VKIIMRSYVGRENVYDIGVERDHNFVAKNGLIAAN

SEQ ID NO: 73 - protein, 102 aa, Synechocystis sp.
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQ

SEQ ID NO: 74 - protein, 35 aa, Synechocystis sp.
VKVIGRRSLGVQRIFDIGLPQDHNFLLANGAIAAN

SEQ ID NO: 75 - protein, 102 aa, Geitlerinema sp.
CLSYETPVMTVEYGPLPIGRIVEEQLDCTVYSVDEQGHVYTQPVAQWHHRGLQEVVEYELEDGRRLRATADHRFMTETGEMLPLAEIFERGLELRQVALRVP

SEQ ID NO: 76 - protein, 35 aa, Geitlerinema sp.
VKIVSRRSLGMQLVYDIGVAADHNFVLADGLIAAN

SEQ ID NO: 77 - protein, 102 aa, Synechocystis sp.
CLSFDAEILTVEYGPLSIGKIVGEEINCSVYSVDPQGRIYTQAIAQWHDRGVQEVFEYELEDGSVIRATPDHRFLTTDYELLAIEEIFARQMDLLTLTNLKL

SEQ ID NO: 78 - protein, 35 aa, Synechocystis sp.
VKVVRRRSLGMHRVFDIGLAQDHNFLLANGAIAAN

SEQ ID NO: 79 - protein, 100 aa, Microcystis aeruginosa
CLGGETLILTEEYGLLPIAKIVSEEVNCTVYSVDKNGFVYSQPISQWHERGLQEVFEYTLENGQTIQATKDHKFMTNDGEMLAIDTIFERGLDLKSSDFS

SEQ ID NO: 80 - protein, 35 aa, Microcystis aeruginosa
VKIISRQSLGRKPVYDIGVEKDHNFLLGNGLIASN

SEQ ID NO: 81 - protein, 100 aa, Microcystis aeruginosa
CLGGETLILTEEYGLLPIAKIVSEEINCTVYTVDQNGFVYSQPISQWHERGLQEVFEYTLENGQTIQATKDHKFMTSDGEMLAIDTIFERGLDLKSSDFS

SEQ ID NO: 82 - protein, 35 aa, Microcystis aeruginosa
VKIIGRQSLGRKPVYDIGVEKDHNFLLGNGLIASN

SEQ ID NO: 83 - protein, 102 aa, Acaryochloris marina
CLSYDTPVLTLEYGWLPIGQVVQEQIECQVFSINERGHLYTQPIAQWHHRGQQEVFEYTLADGSTIQATAEHQFMTTDGQMYPVQQIFEEGLSLKQLPLPWQ

SEQ ID NO: 84 - protein, 35 aa, Acaryochloris marina
VKIIQRRSLGLQSVYDIGLAQDHNFVMANGWVAAN

SEQ ID NO: 85 - protein, 102 aa, Leptolyngbya sp.
CLDGETPIVTVEYGVLPIREIVEKELLCSVYSIDENGFVYTQPVEQWHQRGDRQMFEYQLDNGGVIRATPDHKFLTTEGEMVAIDEIFEKGLNLAEFAPADL

SEQ ID NO: 86 - protein, 35 aa, Leptolyngbya sp.
VKILRRHSIGKAKTYDIGVSKNHNFLLANGLFASN

SEQ ID NO: 87 - protein, 102 aa, Synechococcus elongatus
CLAADTEVLTVEYGPIAIGKLVEENIRCQVYCCNPDGYIYSQPIGQWHQRGEQEVIEYELSDGRIIRATADHRFMTEEGEMLSLDEIFERSLELKQIPTPLL

SEQ ID NO: 88 - protein, 35 aa, Synechococcus elongatus
VKIVRRRSLGVQPVYDLGVATVHNFVLANGLVASN

SEQ ID NO: 89 - protein, 102 aa, Synechococcus sp.
CLSADTELYTVEYGWLPIGRLVEEQIECQVLSVNAHGHVYSQPIAQWHRRAWQEVFEYQLETGGTIKATTDHQFLTTDGQMYRIEDIFQRGLDLWQLPPDRF

SEQ ID NO: 90 - protein, 35 aa, Synechococcus sp.
VKIISRCSLGIQPVYDIGVAQDHNFVIRGGLVASN

SEQ ID NO: 91 - protein, 102 aa, Thermosynechococcus elongatus
CLSGETAVMTVEYGAVPIRRLVQERLSCHVYSLDGQGHLYTQPIAQWHFQGFRPVYEYQLEDGSTICATPDHRFMTTRGQMLPIEQIFQEGLELWQVAIAPR

SEQ ID NO: 92 - protein, 35 aa, Thermosynechococcus elongatus
GKIVGRRLMGWQAVYDIGLAADHNFVLANGAIAAN

SEQ ID NO: 93 - protein, 102 aa, Thermosynechococcus sp.
CLSGETAVMTVEYGAVPIRRLVQERLTCHVYSLDAQGHLYTQPIAQWHFQGFRPVYEYQLEDGSTIWATPDHRFMTTRGQMLPIEQIFQEGLELWQGPIAPS

SEQ ID NO: 94 - protein, 35 aa, Thermosynechococcus sp.
CKIVGRQLVGWQAVYDIGVARDHNFLLANGAIAAN

SEQ ID NO: 95 - protein, 102 aa, Thermosynechococcus vulcanus
CLSGETAVMTVEYGAIPIRRLVQERLICQVYSLDPQGHLYTQPIAQWHFQGFRPVYAYQLEDGSTICATPDHRFMTTSGQMLPIEQIFREGLELWQVAIAPP

SEQ ID NO: 96 - protein, 35 aa, Thermosynechococcus vulcanus
CKIVGRRLVGWQAVYDIGLAGDHNFLLANGAIAAN

SEQ ID NO: 97 - protein, 102 aa, Synechococcus sp.
CLAGGTPVVTVEYGVLPIQTIVEQELLCHVYSVDAQGLIYAQLIEQWHQRGDRLLYEYELENGQMIRATPDHRFLTTTGELLPIDEIFTQNLDLAAWAVPDS

SEQ ID NO: 98 - protein, 35 aa, Synechococcus sp.
VKIIRRKFIGHAPTYDIGLSQDHNFLLGQGLIAAN

SEQ ID NO: 99 - protein, 101 aa, Scytonema hofmanni
CLSYDTEVLTAEYGFLPIGKIVEKAIECTVYSVDNDGNIYTQPIAQWHDRGQQEVFEYSLDDGSVIRATKDHKFMTTGGQMLPIDEIFERGLDLMRIDSLP

SEQ ID NO: 100 - protein, 35 aa, Scytonema hofmanni
VKILTRKSIGKQTVYDIGVERDHNFVIKNGLVASN

SEQ ID NO: 101 - protein, 101 aa, Westiella intricata
CLSYDTEILTVEYGFLPIGEIVEKRIECTVYTVDTNGYVYTQAIAQWHNRGEQEVFEYALEDGSIIRATKDHKFMTSEGQMLPIDEIFVKGLDLLQVQGLP

SEQ ID NO: 102 - protein, 35 aa, Westiella intricata
VKIITRKFLGIQNVYDIGVEQNHNFVIKNGLVASN

SEQ ID NO: 103 - protein, 101 aa, Fischerella sp.
CLSYDTEILTVEYGFLPIGEIVEKGIECTVYTVDNNGNVYTQTIAQWHNRGQQEVFEYCLEDGSVIRATKDHKFMTTDGQMLPIDEIFARGLDLLQVKNLP

SEQ ID NO: 104 - protein, 35 aa, Fischerella sp.
VKIVTRRPLGTQNVYDIGVESDHNFVIKNGLVASN

SEQ ID NO: 105 - protein, 102 aa, Mastigocladopsis repens
CLSYDTEVLTVEYGFLPIGEIVEKSIECSVYTVDSNGNVYTQPIAQWHNRGQQEVFEYCLEDGSIIRATKDHKFMTIHGQMLPIDEIFERGLELMKIQGLPE

SEQ ID NO: 106 - protein, 35 aa, Mastigocladopsis repens
AKIITRKSLGTQNVYDIGVERDHNFVTRDGFIASN

SEQ ID NO: 107 - protein, 102 aa, Scytonema hofmanni
CLSYNSEVLTVEYGFLPIGKIVEKGIECSVYSVDSYGKIYTQVIAQWHNRGQQEVFEYCLEDGTIIQATKDHKFMTVDGQMLPIDEIFERGLDLMQVQGLPD

SEQ ID NO: 108 - protein, 35 aa, Scytonema hofmanni
VKIITRKSLGTQNVYDIGVSSDHNFVMKNGLIASN

SEQ ID NO: 109 - protein, 102 aa, Anabaena sp.
CLSSDTEVLTVEYGLIPIGEIIEKRIDCSVFSVDKNGNIYTQPIAQWHDRGIQELYEYCLDDGSTIRATKDHKFMTTAGEMLPIDEIFERGLDLLKVHNLPQ

SEQ ID NO: 110 - protein, 35 aa, Anabaena sp.
VKIITRNYVGKENVYDIGVERDHNFAIKNGLIASN

SEQ ID NO: 111 - protein, 102 aa, Fischerella sp.
CLSYDTEVLTVEYGFLPIGEIVEKRIECTVYTVDHNGYVYTQPIAQWHNRGYQEVFEYGLEDGSVIRATKDHKFMTSEGQMLPIDEIFARELDLLQVTGLVN

SEQ ID NO: 112 - protein, 35 aa, Fischerella sp.
VKIVTRRLLGIQNVYDIGVEQNHNFVIKNGLVASN

SEQ ID NO: 113 - protein, 102 aa, Calothrix sp.
CLSYDTEIFTVEYGFLPIGEIVEKRLECTVLTVDNHGNIYSQPIAQWHHRGQQQIYEYGLEDGSVIRATKDHKFMTTDGQMLPIDEIFERGLDLLQVTNLDN

SEQ ID NO: 114 - protein, 35 aa, Calothrix sp.
VKVITRKLADTENVYDIGVENHHNFLIKNGLVASN

SEQ ID NO: 115 - protein, 101 aa, Fischerella thermalis
CLSYETEILTVEYGFLPIGEIVEKRIECSVYTVDNNGYVCTQPIAQWHNRGYQEVFEYGLEDGSVIRATKDHKFMTIDRQMLPIDEIFARGLDLLQVTGLP

SEQ ID NO: 116 - protein, 35 aa, Fischerella thermalis
VKIITRKSLGTQNVYDIGVEQNHNFVIKNGLVASN

SEQ ID NO: 117 - protein, 102 aa, Cyanobacterium sp.
CLSYDTEILTVEYGFLSIGEIVEKEIECTVYTVDSNGYIYTQPIAQWHEQGEQEIFEYSLEDGSTIRATKDHKFMTIEGEMLPIDQIFARQLDLMQITGLPQ

SEQ ID NO: 118 - protein, 35 aa, Cyanobacterium sp.
VKISTKKSLGKQKVYDIGVVRDHNFIIKNGFVASN

SEQ ID NO: 119 - protein, 101 aa, Fischerella sp.
CLSYDTEVLTVEYGFLPIGEIVEKRIECTVYTVDTNGYVYTQAIAQWHNRDEQEVFEYALEDGSIIRATKDHKFMTSEGQMLPIDEIFAKGLDLLQVQGLP

SEQ ID NO: 120 - protein, 35 aa, Fischerella sp.
VKIVTRKFLGIQNVYDIGVEQNHNFVIKNGLVASN

SEQ ID NO: 121 - protein, 102 aa, Fischerella muscicola
CLSYETEILTVEYGFLPIGEIVEKRIECSVYTVDNNGYVCTQTIAQWHNRGYQEVFEYGLEDGSVIRATKDHKFMTIDRQMLPIDEIFARGLDLLQVKGLPE

SEQ ID NO: 122 - protein, 35 aa, Fischerella muscicola
VKIITRQSLGTQNVYDIGVEQNHNFVIKNGLVASN

SEQ ID NO: 123 - protein, 102 aa, Fischerella muscicola
CLSYDTEVLTVEYGFLPIGEIVEKTIECNVFTVDSNGYVYTQPIAQWHNRGYQEVFEYGLEDGSVIRATKDHKFMTSEGKMLPIDEIFARELDLLQVTGLIN

SEQ ID NO: 124 - protein, 35 aa, Fischerella muscicola; identical to SEQ ID NO: 120

SEQ ID NO: 125 - protein, 102 aa, Lyngbya aestuarii
CLSYDTEILTVEYGAIPIGKVVDEKIECTVYSVDKNGLIYTQPIAQWHNRGKQEVFEYSLEDGSTIRATKDHKFMTMDNQMLPIDEILEKGLELKQVNADSV

SEQ ID NO: 126 - protein, 35 aa, Lyngbya aestuarii
VKIVSRKSLDSQTVYDIGVETDHNFLLANGSVASN

SEQ ID NO: 127 - protein, 100 aa, Microchaete sp.
CLSYKTQVLTVEYGLLAIGEIVEKNIECSVFSVDIHGNVYTQPIAQWHHRGQQEVFEYGLEDGSIIRATKDHKFMTTQGEMLPIDEIFARGLDLLQVKGV

SEQ ID NO: 128 - protein, 35 aa, Microchaete sp.
VKIITRKYIGKENVYDIGVEQDHNFAIKNGLIAAN

SEQ ID NO: 129 - protein, 102 aa, Leptolyngbya sp.
CLSYDTEILTVEYGALPIGKIVENQMICSVYSIDNNGYIYIQPIAQWHNRGQQEVFEYILEDGSIIRSTKDHKFMTKGGEMLPIDEIFERGLELAQVTRLEQ

SEQ ID NO: 130 - protein, 35 aa, Leptolyngbya sp.
VKIISRRSVGVQSVYDIGVKQDHNFFLRNGLIASN

SEQ ID NO: 131 - protein, 102 aa, Crocosphaera watsonii
CLSYDTEILTVEYGAMYIGKIVEENINCTVYTVDKNGFVYTQTIAQWHNRGEQEIFEYDLEDGSKIKATKDHKFMTIDGEMLPIDEIFEKNLDLKQVVSHPD

SEQ ID NO: 132 - protein, 35 aa, Crocosphaera watsonii
VKIIGCRSLGTQKVYDIGVEKDHNFLLANGSIASN

SEQ ID NO: 133 - protein, 100 aa, Coleofasciculus chthonoplastes
CLSYDTQILTVEYGAVAIGEIVEKQIECTVYSVDENGYVYTQPIAQWHNRGEQEVFEYLLEDGATIRATKDHKFMTDEDQMLPIDQIFEQGLELKQVEVL

SEQ ID NO: 134 - protein, 35 aa, Coleofasciculus chthonoplastes
VKIIGRKPLGTQPVYDIGVERDHNFLLFNGSVASN

SEQ ID NO: 135 - protein, 98 aa, Chroococcidiopsis sp.
CLSYDTEVLTVEYGAIPIGKIVEEKIACNVYSVDKNGFVYTQPIAQYHDRGIQEVFEYRLENGSVIRATKDHKMMTADGQMLPIDEIFKQNLDLKQLN

SEQ ID NO: 136 - protein, 35 aa, Chroococcidiopsis sp.
VKIISRQSLGKQSVFDIGVAKDHNFLLANGLVASN

SEQ ID NO: 137 - protein, 97 aa, Aphanizomenon flos-aquae
CLSYDTEILTVEYGFLQIGEIVEKQIECKVYTVDSNGILYTQSIAQWHNRGQQEVYEYLLENGAIIRATKDHKFMTEEGQMLPIDEIFSQGLDLLQV

SEQ ID NO: 138 - protein, 35 aa, Aphanizomenon flos-aquae
VKIISRTYVGQANVYDIGVENDHNFVIKNGFIAAN

SEQ ID NO: 139 - protein, 102 aa, Raphidiopsis brookii
CLSYETEVLTLEYGFLPIGEIVDKQMVCTVFSVNDSGNVYTQPIGQWHDRGVQELYEYCLDDGSTIRATKDHKFMTTQGEMVPIDEIFHQGWELVQVSGTMN

SEQ ID NO: 140 - protein, 35 aa, Raphidiopsis brookii
VKIVSRRYLGKADVYDIGVAKDHNFIIKNGLVASN

SEQ ID NO: 141 - protein, 99 aa, Cyanothece sp.
CLSYDTEILTVEYGPMPIGKIVEENINCSVYTVNKNGFVYTQSIAQWHHRGEQEVFEYYLEDGETIRATKDHKFMTTEGKMLPIDEIFENNLDLKKLTV

SEQ ID NO: 142 - protein, 35 aa, Cyanothece sp.
VKIIERRSLGKQNVYDIGVEKDHNFLLSNNLIASN

SEQ ID NO: 143 - protein, 100 aa, Xenococcus sp.
CLSADTEVLTVEYGAISIGKIVEERIECTVYSVDANGFVYTQEIAQWHNRGEQEVFEYMLDDGSVIRATKDHKLMTIDGQMVAIDEIFSQGLELKQVLGL

SEQ ID NO: 144 - protein, 35 aa, Xenococcus sp.
VKIVSRKSLGTQTVYDLGVARDHNFLLANGTVASN

SEQ ID NO: 145 - protein, 100 aa, Pleurocapsa sp.
CLSYDTEIYTVEYGALPIGKIVESRIKCTVLTVDKNGLVYSQPIVQWHDRGIQEVFEYTLDNGATIRATKDHKFMTVEGQMLPIDEIFELGLELKEIQQF

SEQ ID NO: 146 - protein, 35 aa, Pleurocapsa sp.
VKIISRQSLGKQSVYDIGVAKDHNFLLANGMVASN

SEQ ID NO: 147 - protein, 102 aa, Cylindrospermopsis raciborskii
CLSYETEVLTLEYGFVPIGEIVNKQMVCTVFSLNDSGNVYTQPIGQWHDRGVQDLYEYCLDDGSTIRATKDHKFMTTQGEMVPIDEIFHQGWELVQVSGISK

SEQ ID NO: 148 - protein, 35 aa, Cylindrospermopsis raciborskii; identical to SEQ ID NO: 140

SEQ ID NO: 149 - protein, 94 aa, Spirulina major
CLTYDTLVLTVEYGPVPIGKLVEAQINCQVYSVDANGFIYTQAIAQWHDRGQRQVYEYTLEDGSTIRATPDHKFMTATGEMLPIDQIFEQGLDL

SEQ ID NO: 150 - protein, 35 aa, Spirulina major
VKIIHRRALPPQSVYDIGVERDHNFLLPSGWVASN

SEQ ID NO: 151 - protein, 96 aa, Spirulina subsalsa
CLSYDTKIITVEYGAIAIGTIVEQGLHCHVYSVDPNGFIYTQPIAQWHQRGEQEVFAYTLENGSIIQATKDHKFMTQQGKMLPIDTIFEQGLDLLQ

SEQ ID NO: 152 - protein, 35 aa, Spirulina subsalsa
VKIIKRTSLGVRPVYDIGVIQDHNFLLENGLVASN

SEQ ID NO: 153 - protein, 100 aa, Microcystis aeruginosa
CLGGETLILTEEYGLLPIAKIVSEEINCTVYSVDKNGFIYSQPISQWHERGLQEVFEYTLENGQTIQATKDHKFMTSDGEMLAIDTIFERGLDLKSSDFS

SEQ ID NO: 154 - protein, 35 aa, Microcystis aeruginosa
VKIISRQFLGRKPVYDIGVEKDHNFLLGNGLIASN

SEQ ID NO: 155 - protein, 95 aa, Myxosarcina sp.
CLSYDTEVLTLKYGALPIGEIVEKRINCHVYTRAESGFFYIQSIEQWHDRGEQEVFEYTLENGATIKATKDHKFMTSGGQMLPIDEIFERGLDLL

SEQ ID NO: 156 - protein, 35 aa, Myxosarcina sp.
VKIVSRKSLGKQPVYDLGVAKDHNFLLANGTVASN

SEQ ID NO: 157 - protein, 101 aa, Leptolyngbya sp.
CLSADTQLLTVEYGPLEIGRIVEEQIACHVYSVDANGFVYTQPIAQWHSRGEQEIFEYQLEDGRTLRATADHKFMTTTGEMGRINDIFEQGLDLKQIDLPQ

SEQ ID NO: 158 - protein, 35 aa, Leptolyngbya sp.
VKVVSRQSLGVQPVYDIGVATDHNFLLADGLVASN

SEQ ID NO: 159 - protein, 97 aa, Acaryochloris sp.
CLSYDTPVLTLEYGWLPIGQVVQEQIECQVFSINERGHLYTQPIAQWHHRGQQEVFEYTLTDGSTIQATAEHQFMTTDGQMYPIQQIFEEGLSLKQL

SEQ ID NO: 160 - protein, 35 aa, Acaryochloris sp.
VKITQRRSLGLQSVYDIGLAQDHNFVIANGWVAAN

SEQ ID NO: 161 - protein, 98 aa, Geminocystis herdmanii
CLSYDTEVLTVEFGAIPMGKIVEERLNCQVYSVDKNGFIYTQNIAQWHDRGVQEVFEYELEDGRIIKATKDHKMMIENCEMVEIDRIFEEGLELFEVN

SEQ ID NO: 162 - protein, 35 aa, Geminocystis herdmanii
VKILKRRSISSQQVYDIGVEKDHNFLLANGLVASN

SEQ ID NO: 163 - protein, 98 aa, Nodosilinea nodulosa
CLSADTELLTLEYGPLTIGEIVAKRIPCHVFSVDESGYVYTQPVAQWHQRGHQEVFEYQLDDGTTIRATADHQFMTELGEMMAIDEIFQRGLELKQVE

SEQ ID NO: 164 - protein, 35 aa, Nodosilinea nodulosa
VKIISRQSLGVQPVYDIGVARDHNFLLADGQVASN

SEQ ID NO: 165 - protein, 102 aa, Rubidibacter lacunae
CLSYDTEVLTVEYGPLAIGTIVSERLACTVYTVDRSGFLYAQAISQWHERGRQDVFEYALDNGMTIRATKDHKLMTADGQMVAIDDIFTQGLTLKAIDTAAF

SEQ ID NO: 166 - protein, 35 aa, Rubidibacter lacunae
MKIVSRKSLGVQHVYDIGVARDHNFLLANGAIASN

SEQ ID NO: 167 - protein, 101 aa, Chlorogloeopsis fritschii
CLSYDTAILTVEYGFLPIGEIVEKGIECTVYTVDSNGYIYTQPIAQWHNRGEQELFEYSLEDGSIIRATKDHKFMTIDGQMLPIDEIFARKLELMQVKGLP

SEQ ID NO: 168 - protein, 35 aa, Chlorogloeopsis fritschii
VKIIAKKSLGTQNVYDIGVERDHNFVIKNGLVASN

SEQ ID NO: 169 - protein, 102 aa, Richelia intracellularis
CLSYDTQILTVEHGPMSIGEIVEKCLECHVYTVNKNGNICIQTITQWHFRGEQEIFEYELEDGSFIQATKDHKFMTTTGEMLPIHEIFTNGLEILQLSKSLL

SEQ ID NO: 170 - protein, 35 aa, Richelia intracellularis
VKILARKSLGTQKVYDIGVNDDHNFALSNSFIASN

SEQ ID NO: 171 - protein, 102 aa, Synechococcus sp.
CLAGDTPVVTVEYGVLPIQTIVEQELLCQVYSVDAQGLIYTQPIEQWHNRGDRLLYEYELENGQMIRATPDHKFLTTTGELLPIDEIFTQNLDLAAWAVPDS

SEQ ID NO: 172 - protein, 35 aa, Synechococcus sp.; identical to SEQ ID NO: 98

SEQ ID NO: 173 - protein, 102 aa, Synechococcus sp.
CLAGDTPVVTVEYGVLPIQTIVEQELLCHVYSVDAQGLIYTQPIEQWHQRGDRFLYEYELENGQMIRATPDHKFLTTTGKLLPIDEIFTQNLDLAAWAVPDS

SEQ ID NO: 174 - protein, 35 aa, Synechococcus sp.
VKIIRRKFIGHAPTYDIGLSQDHNFLLGQGFIAAN

SEQ ID NO: 175 - protein, 102 aa, Synechococcus sp.
CLAGDTPVVTVEYGVLPIQTIVEQELLCHVYSVDAQGLIYTQPIEQWHQRGDRLLYEYELENGQMIRATPDHKFLTTTGELLPIDEIFTQNLDLAAWAVPDS

SEQ ID NO: 176 - protein, 35 aa, Synechococcus sp.
VKILRRKFIGRAPTYDIGLSQDHNFLLGQGLVAAN

SEQ ID NO: 177 - protein, 94 aa, Synechococcus sp.
CLAGDTPVVTVEYGVLPIRTIVDQELLCHVYSLDPQGFIYAQPVEQWHRRGDRLLYEYELETGAVIRATPDHKFLTATGEMLPIDEIFVRNLDL

SEQ ID NO: 178 - protein, 35 aa, Synechococcus sp.
VKIIRRNLIGEAATYDIGLGKDHNFLLGQGLIASN

SEQ ID NO: 179 - protein, 95 aa, Synechococcus sp.
CLAGGTPVVTVEYGVLPIQTIVEQELLCHVYSVDAQGLIYTQPIEQWHQRGDRLLYEYELENGQMIRATPDHKFLTTTGELLPIDEIFTQNLDLL

SEQ ID NO: 180 - protein, 35 aa, Synechococcus sp.; identical to SEQ ID NO: 98

SEQ ID NO: 181 - protein, 95 aa, Synechococcus sp.
CLAGDTPVVTVEYGVLPIQTIVEQELLCHVYSVDAQGLIYTQPIEQWHKRGDRLLYEYELENGQIIRATPDHKFLTTTGEMRPIDEIFAKNLSLL

SEQ ID NO: 182 - protein, 35 aa, Synechococcus sp.
VKIIRRKFVGHAPTYDIGLSQDHNFLLGQGLIAAN

SEQ ID NO: 183 - protein, 99 aa, Cyanothece sp.; identical to SEQ ID NO: 51

SEQ ID NO: 184 - protein, 35 aa, Cyanothece sp.; identical to SEQ ID NO: 52

SEQ ID NO: 185 - protein, 102 aa, Synechococcus elongatus; identical to SEQ ID NO: 87

SEQ ID NO: 186 - protein, 35 aa, Synechococcus elongatus; identical to SEQ ID NO: 88

SEQ ID NO: 187 - protein, 102 aa, Chlorogloeopsis fritschii
CLSYDTAILTVEYGFLPIGEIVEKGIECTVYTVDSNGYIYTQPIAQWHNRGEQELFEYSLEDGSIIRATKDHKFMTIDGQMLPIDEIFARKLELMQVKGLPE

SEQ ID NO: 188 - protein, 35 aa, Chlorogloeopsis fritschii; identical to SEQ ID NO: 168

SEQ ID NO: 189 - protein, 97 aa, Cyanothece sp.
CLSYDTEILTVEYGPMPIGKIVEENINCTVYTVDPNGFVYTQAIAQWHYRGEQEIFEYYLEDGATIRATKDHKFMTMEGKMLPIDEIFENNLDLKQL

SEQ ID NO: 190 - protein, 35 aa, Cyanothece sp.; identical to SEQ ID NO: 50

SEQ ID NO: 191 - protein, 97 aa, Lyngbya majuscula
CLSYDTEIITVEYGPIAIGEIVEKGIPCTVYSVDSNGYVYTQPIAQWHNRGEQEVFEYTLDDGSVIRATKDHKFMTIDGQMLPIDEIFEGGLELKQL

SEQ ID NO: 192 - protein, 35 aa, Lyngbya majuscula
VKIISRKSLGTQPVYDIGVKDDHNFILANGMVASN

SEQ ID NO: 193 - protein, 102 aa, unidentified
  source 1..102: note = filamentous cyanobacterium ESFC-1 sequence
CLSYDTEVLTVEYGAVPIGKLVEEKLNCSVYTVDPNGYIYTQAIAQWHDRGIQEVFEYQLEDNTIIRATKDHKFMTEDHQMLPIDEIFERGLELKKCPQPQQ

SEQ ID NO: 194 - protein, 35 aa, unidentified
  source 1..35: note = filamentous cyanobacterium ESFC-1 sequence
VKIIRRRSLGFQPVYDIGLEQDHNFLLNQGAIASN

SEQ ID NO: 195 - protein, 94 aa, Synechococcus sp.
CLAGGTPVVTVEYGVLPIQTIVEQELLCHVYSVDAQGLIYAQLIEQWHQRGDRLLYEYELENGQMIRATPDHRFLTTTGELLPIDEIFTQNLDL

SEQ ID NO: 196 - protein, 35 aa, Synechococcus sp.; identical to SEQ ID NO: 98

SEQ ID NO: 197 - protein, 97 aa, Acaryochloris marina
CLSYDTPVLTLEYGWLPIGQVVQEQIECQVFSINERGHLYTQPIAQWHHRGQQEVFEYTLADGSTIQATAEHQFMTTDGQMYPVQQIFEEGLSLKQL

SEQ ID NO: 198 - protein, 35 aa, Acaryochloris marina; identical to SEQ ID NO: 84

SEQ ID NO: 199 - protein, 94 aa, Microcystis aeruginosa
CLGGETLILTEEYGLLPIAKIVSEEVNCTVYSVDKNGFVYSQPISQWHERGLQEVFEYTLENGQTIQATKDHKFMTNDGEMLAIDTIFERGLDL

SEQ ID NO: 200 - protein, 35 aa, Microcystis aeruginosa; identical to SEQ ID NO: 80

SEQ ID NO: 201 - protein, 90 aa, Anabaena circinalis
CLSYDTEILTVEYGFLEIGEIVEKQIECKVYTVDSNGILYTQPIAQWHHRGQQEVYEYLLENGAIIRATKDHKFMTEAGEMLPIDDIFTQ

SEQ ID NO: 202 - protein, 35 aa, Anabaena circinalis
VKIISRTYVGQANVYDIGVENDHNFVIKNGFVAAN

SEQ ID NO: 203 - protein, 90 aa, Anabaena circinalis
CLSYDTEILTVEYGFLEIGEIVEKQIECRVYTVDSNGILYTQPIAQWHYRGQQEVYEYLLENGAIIRATKDHNFMTEAGEMLPIDDIFTQ

SEQ ID NO: 204 - protein, 35 aa, Anabaena circinalis
IKIISRKYVGQANVYDIGVENDHNFVIKNGFVAAN

SEQ ID NO: 205 - protein, 90 aa, Cyanobacterium sp.
CLSYDTKVLTVEYGPLPIGKVVQENIRCRVYTTNDQGLIYTQPIAQWHNRGKQEIFEYHLDDKTIIRATKEHQFMTVDHVMMPIDEIFEQ

SEQ ID NO: 206 - protein, 34 aa, Cyanobacterium sp.
KIIRRKSLGMHEVFDIGLEKDHNFVLSNGLIASN

SEQ ID NO: 207 - protein, 94 aa, Planktothrix sp.
CLSYDTEVLTVEYGLIPISKIVEEKIECTVYTVNNQGYVYTQPIAQWHNRGEQEVFEYYLEDGSVIRATKDHKFMTVEGQMLPIDEIFEKELDL

SEQ ID NO: 208 - protein, 35 aa, Planktothrix sp.
VKIISRKSLGTQPVYDIGVQEDHNFVLNNGLVASN

SEQ ID NO: 209 - protein, 94 aa, Planktothrix sp.
CLSYDTEILTVEYGLMPIGKIVKEKIECTVYTVNNQGYVYTQPIAQWHHRGEQEVFEYCLEDGSVIRATKDHKFMTVQGQMLPIDEIFEKELDL

SEQ ID NO: 210 - protein, 35 aa, Planktothrix sp.
VKIISRKSLGTQPVYDIGVQEDHNFLLNNGLVASN

SEQ ID NO: 211 - protein, 102 aa, Fremyella diplosiphon
CLSYDTEVLTVEYGLIPIGEIVEKRLECSVYSVDINGNVYTQPIAQWHHRGQQEVFEYALEDGSIIRATKDHKFMTTDGQMLPIDEIFERGLDLLQVPHLPE

SEQ ID NO: 212 - protein, 35 aa, Fremyella diplosiphon
VKIVTRRAIGAANVYDIGVEQDHNFAIKNGLIAAN

SEQ ID NO: 213 - protein, 94 aa, Planktothrix sp.
CLSYDTEILTVEYGLIPISKIVEEKIECTVYTVNNQGYVYTQPIAQWHNRGEQEVFEYYLEDGSVIRATKDHKFMTVDGQMLPIDEIFEKELDL

SEQ ID NO: 214 - protein, 35 aa, Planktothrix sp.; identical to SEQ ID NO: 208

SEQ ID NO: 215 - protein, 102 aa, Nostoc punctiforme; identical to SEQ ID NO: 5

SEQ ID NO: 216 - protein, 35 aa, Nostoc punctiforme; identical to SEQ ID NO: 6

SEQ ID NO: 217 - protein, 102 aa, Chroococcidiopsis thermalis; identical to SEQ ID NO: 7

SEQ ID NO: 218 - protein, 35 aa, Chroococcidiopsis thermalis; identical to SEQ ID NO: 8

SEQ ID NO: 219 - protein, 102 aa, Nodularia spumigena; identical to SEQ ID NO: 9

SEQ ID NO: 220 - protein, 35 aa, Nodularia spumigena; identical to SEQ ID NO: 10

SEQ ID NO: 221 - protein, 102 aa, Anabaena cylindrica; identical to SEQ ID NO: 11

SEQ ID NO: 222 - protein, 35 aa, Anabaena cylindrica; identical to SEQ ID NO: 12

SEQ ID NO: 223 - protein, 102 aa, Calothrix sp.; identical to SEQ ID NO: 13

SEQ ID NO: 224 - protein, 35 aa, Calothrix sp.; identical to SEQ ID NO: 14

SEQ ID NO: 225 - protein, 102 aa, Nostoc sp.; identical to SEQ ID NO: 15

SEQ ID NO: 226 - protein, 35 aa, Nostoc sp.; identical to SEQ ID NO: 16

SEQ ID NO: 227 - protein, 102 aa, Nostoc azollae; identical to SEQ ID NO: 17

SEQ ID NO: 228 - protein, 35 aa, Nostoc azollae; identical to SEQ ID NO: 18

SEQ ID NO: 229 - protein, 102 aa, Nostoc sp.; identical to SEQ ID NO: 19

SEQ ID NO: 230 - protein, 35 aa, Nostoc sp.; identical to SEQ ID NO: 20

SEQ ID NO: 231 - protein, 102 aa, Anabaena variabilis; identical to SEQ ID NO: 21

SEQ ID NO: 232 - protein, 35 aa, Anabaena variabilis; identical to SEQ ID NO: 22

SEQ ID NO: 233 - protein, 100 aa, Pleurocapsa sp.; identical to SEQ ID NO: 23

SEQ ID NO: 234 - protein, 35 aa, Pleurocapsa sp.; identical to SEQ ID NO: 24

SEQ ID NO: 235 - protein, 100 aa, Cyanothece sp.; identical to SEQ ID NO: 25

SEQ ID NO: 236 - protein, 35 aa, Cyanothece sp.; identical to SEQ ID NO: 26

SEQ ID NO: 237 - protein, 99 aa, Cyanothece sp.; identical to SEQ ID NO: 27

SEQ ID NO: 238 - protein, 35 aa, Cyanothece sp.; identical to SEQ ID NO: 28

SEQ ID NO: 239 - protein, 102 aa, Nostoc sp.; identical to SEQ ID NO: 29

SEQ ID NO: 240 - protein, 35 aa, Nostoc sp.; identical to SEQ ID NO: 30

SEQ ID NO: 241 - protein, 101 aa, Tolypothrix bouteillei; identical to SEQ ID NO: 31

SEQ ID NO: 242 - protein, 35 aa, Tolypothrix bouteillei; identical to SEQ ID NO: 32

SEQ ID NO: 243 - protein, 101 aa, Aphanizomenon ovalisporum; identical to SEQ ID NO: 33
  VAR_SEQ 87: note = Any amino acid

SEQ ID NO: 244 - protein, 35 aa, Aphanizomenon ovalisporum; identical to SEQ ID NO: 34

SEQ ID NO: 245 - protein, 102 aa, Oscillatoria nigro-viridis; identical to SEQ ID NO: 35

SEQ ID NO: 246 - protein, 35 aa, Oscillatoria nigro-viridis; identical to SEQ ID NO: 36

SEQ ID NO: 247 - protein, 100 aa, Rivularia sp.; identical to SEQ ID NO: 37

SEQ ID NO: 248 - protein, 35 aa, Rivularia sp.; identical to SEQ ID NO: 38

SEQ ID NO: 249 - protein, 102 aa, Microcoleus sp.; identical to SEQ ID NO: 41

SEQ ID NO: 250 - protein, 35 aa, Microcoleus sp.; identical to SEQ ID NO: 42

SEQ ID NO: 251 - protein, 102 aa, Stanieria cyanosphaera; identical to SEQ ID NO: 43

SEQ ID NO: 252 - protein, 35 aa, Stanieria cyanosphaera; identical to SEQ ID NO: 44

SEQ ID NO: 253 - protein, 102 aa, Calothrix sp.; identical to SEQ ID NO: 45

SEQ ID NO: 254 - protein, 35 aa, Calothrix sp.; identical to SEQ ID NO: 38

SEQ ID NO: 255 - protein, 99 aa, Cyanobacterium stanieri; identical to SEQ ID NO: 47

SEQ ID NO: 256 - protein, 35 aa, Cyanobacterium stanieri; identical to SEQ ID NO: 48

SEQ ID NO: 257 - protein, 99 aa, Cyanothece sp.; identical to SEQ ID NO: 49

SEQ ID NO: 258 - protein, 35 aa, Cyanothece sp.; identical to SEQ ID NO: 50

SEQ ID NO: 259 - protein, 99 aa, Cyanothece sp.; identical to SEQ ID NO: 51

SEQ ID NO: 260 - protein, 35 aa, Cyanothece sp.; identical to SEQ ID NO: 52

SEQ ID NO: 261 - protein, 101 aa, Anabaena sp.; identical to SEQ ID NO: 53

SEQ ID NO: 262 - protein, 35 aa, Anabaena sp.; identical to SEQ ID NO: 54

SEQ ID NO: 263 - protein, 102 aa, Aphanothece halophytica; identical to SEQ ID NO: 55

SEQ ID NO: 264 - protein, 35 aa, Aphanothece halophytica; identical to SEQ ID NO: 56

SEQ ID NO: 265 - protein, 102 aa, Halothece sp.; identical to SEQ ID NO: 55

SEQ ID NO: 266 - protein, 35 aa, Halothece sp.; identical to SEQ ID NO: 58

SEQ ID NO: 267 - protein, 102 aa, Cyanobacterium aponinum; identical to SEQ ID NO: 59

SEQ ID NO: 268 - protein, 35 aa, Cyanobacterium aponinum; identical to SEQ ID NO: 60

SEQ ID NO: 269 - protein, 98 aa, unidentified; identical to SEQ ID NO: 61
  source 1..98: note = Candidatus Atelocyanobacterium thalassa isolate sequence

SEQ ID NO: 270 - protein, 35 aa, unidentified; identical to SEQ ID NO: 62
  source 1..35: note = Candidatus Atelocyanobacterium thalassa isolate sequence

SEQ ID NO: 271 - protein, 102 aa, Oscillatoria limnetica; identical to SEQ ID NO: 63

SEQ ID NO: 272 - protein, 35 aa, Oscillatoria limnetica; identical to SEQ ID NO: 64

SEQ ID NO: 273 - protein, 102 aa, unidentified; identical to SEQ ID NO: 65
  source 1..102: note = Cyanobacterium endosymbiont of Epithemia turgida sequence

SEQ ID NO: 274 - protein, 35 aa, unidentified; identical to SEQ ID NO: 66
  source 1..35: note = Cyanobacterium endosymbiont of Epithemia turgida sequence

SEQ ID NO: 275 - protein, 98 aa, Synechococcus sp.; identical to SEQ ID NO: 67

SEQ ID NO: 276 - protein, 35 aa, Synechococcus sp.; identical to SEQ ID NO: 68

SEQ ID NO: 277 - protein, 90 aa, Cyanobacterium sp.; identical to SEQ ID NO: 205

SEQ ID NO: 278 - protein, 34 aa, Cyanobacterium sp.; identical to SEQ ID NO: 206

SEQ ID NO: 279 - protein, 94 aa, Planktothrix sp.; identical to SEQ ID NO: 207

SEQ ID NO: 280 - protein, 35 aa, Planktothrix sp.; identical to SEQ ID NO: 208

SEQ ID NO: 281 - protein, 94 aa, Planktothrix sp.; identical to SEQ ID NO: 209

SEQ ID NO: 282 - protein, 35 aa, Planktothrix sp.; identical to SEQ ID NO: 210

SEQ ID NO: 283 - protein, 94 aa, Planktothrix sp.; identical to SEQ ID NO: 213

SEQ ID NO: 284 - protein, 35 aa, Planktothrix sp.; identical to SEQ ID NO: 208

SEQ ID NO: 285 - protein, 99 aa, Cyanothece sp.; identical to SEQ ID NO: 51

SEQ ID NO: 286 - protein, 35 aa, Cyanothece sp.; identical to SEQ ID NO: 52

SEQ ID NO: 287 - protein, 102 aa, Chlorogloeposis fritschii; identical to SEQ ID NO: 187

SEQ ID NO: 288 - protein, 35 aa, Chlorogloeposis fritschii; identical to SEQ ID NO: 168

SEQ ID NO: 289 - protein, 97 aa, Cyanothece sp.; identical to SEQ ID NO: 189

SEQ ID NO: 290 - protein, 35 aa, Cyanothece sp.; identical to SEQ ID NO: 50

SEQ ID NO: 291 - protein, 97 aa, Lyngbya majuscula; identical to SEQ ID NO: 191

SEQ ID NO: 292 - protein, 35 aa, Lyngbya majuscula; identical to SEQ ID NO: 192

SEQ ID NO: 293 - protein, 102 aa, unidentified; identical to SEQ ID NO: 193
  source 1..102: note = filamentous cyanobacterium ESFC-1 sequence

SEQ ID NO: 294 - protein, 35 aa, unidentified; identical to SEQ ID NO: 194
  source 1..35: note = filamentous cyanobacterium ESFC-1 sequence

SEQ ID NO: 295 - protein, 94 aa, Microcystis aeruginosa; identical to SEQ ID NO: 199

SEQ ID NO: 296 - protein, 35 aa, Microcystis aeruginosa; identical to SEQ ID NO: 80

SEQ ID NO: 297 - protein, 102 aa, Rubidibacter lacunae; identical to SEQ ID NO: 165

SEQ ID NO: 298 - protein, 35 aa, Rubidibacter lacunae; identical to SEQ ID NO: 166

SEQ ID NO: 299 - protein, 101 aa, Chlorogloeopsis fritschii; identical to SEQ ID NO: 167

SEQ ID NO: 300 - protein, 35 aa, Chlorogloeopsis fritschii; identical to SEQ ID NO: 168

SEQ ID NO: 301 - protein, 102 aa, Richelia intracellularis; identical to SEQ ID NO: 169

SEQ ID NO: 302 - protein, 35 aa, Richelia intracellularis; identical to SEQ ID NO: 170

SEQ ID NO: 303 - protein, 98 aa, Geminocystis herdmanii; identical to SEQ ID NO: 161

SEQ ID NO: 304 - protein, 35 aa, Geminocystis herdmanii; identical to SEQ ID NO: 162

SEQ ID NO: 305 - protein, 97 aa, Spirulina subsalsa
CLSYDTKIITVEYGAIAIGTIVEQGLHCHVYSVDPNGFIYTQPIAQWHQRGEQEVFAYTLENGSIIQATKDHKFMTQQGKMLPIDTIFEQGLDLLQV

SEQ ID NO: 306 - protein, 34 aa, Spirulina subsalsa
KIIKRTSLGVRPVYDIGVIQDHNFLLENGLVASN

SEQ ID NO: 307 - protein, 100 aa, Microcystis aeruginosa; identical to SEQ ID NO: 153

SEQ ID NO: 308 - protein, 35 aa, Microcystis aeruginosa; identical to SEQ ID NO: 154

SEQ ID NO: 309 - protein, 95 aa, Myxosarcina sp.; identical to SEQ ID NO: 155

SEQ ID NO: 310 - protein, 35 aa, Myxosarcina sp.; identical to SEQ ID NO: 156

SEQ ID NO: 311 - protein, 101 aa, Scytonema hofmanni; identical to SEQ ID NO: 99

SEQ ID NO: 312 - protein, 35 aa, Scytonema hofmanni; identical to SEQ ID NO: 100

SEQ ID NO: 313 - protein, 101 aa, Westiella intricata; identical to SEQ ID NO: 101

SEQ ID NO: 314 - protein, 35 aa, Westiella intricata; identical to SEQ ID NO: 102

SEQ ID NO: 315 - protein, 101 aa, Fischerella sp.; identical to SEQ ID NO: 103

SEQ ID NO: 316 - protein, 35 aa, Fischerella sp.; identical to SEQ ID NO: 104

SEQ ID NO: 317 - protein, 102 aa, Mastigocladopsis repens; identical to SEQ ID NO: 105

SEQ ID NO: 318 - protein, 35 aa, Mastigocladopsis repens; identical to SEQ ID NO: 106

SEQ ID NO: 319 - protein, 102 aa, Scytonema hofmanni; identical to SEQ ID NO: 107

SEQ ID NO: 320 - protein, 35 aa, Scytonema hofmanni; identical to SEQ ID NO: 108

SEQ ID NO: 321 - protein, 102 aa, Anabaena sp.; identical to SEQ ID NO: 109

SEQ ID NO: 322 - protein, 35 aa, Anabaena sp.; identical to SEQ ID NO: 110

SEQ ID NO: 323 - protein, 102 aa, Fischerella sp.; identical to SEQ ID NO: 111

SEQ ID NO: 324 - protein, 35 aa, Fischerella sp.; identical to SEQ ID NO: 112

SEQ ID NO: 325 - protein, 102 aa, Calothrix sp.; identical to SEQ ID NO: 113

SEQ ID NO: 326 - protein, 35 aa, Calothrix sp.; identical to SEQ ID NO: 114

SEQ ID NO: 327 - protein, 101 aa, Fischerella thermalis; identical to SEQ ID NO: 115

SEQ ID NO: 328 - protein, 35 aa, Fischerella thermalis; identical to SEQ ID NO: 116

SEQ ID NO: 329 - protein, 102 aa, unidentified; identical to SEQ ID NO: 117
  source 1..102: note = cyanobacterium PCC 7702 Chl7702 sequence

SEQ ID NO: 330 - protein, 35 aa, unidentified; identical to SEQ ID NO: 118
  source 1..35: note = cyanobacterium PCC 7702 Chl7702 sequence

SEQ ID NO: 331 - protein, 101 aa, Fischerella sp.; identical to SEQ ID NO: 119

SEQ ID NO: 332 - protein, 35 aa, Fischerella sp.; identical to SEQ ID NO: 120

SEQ ID NO: 333 - protein, 102 aa, Fischerella muscicola; identical to SEQ ID NO: 121

SEQ ID NO: 334 - protein, 35 aa, Fischerella muscicola; identical to SEQ ID NO: 122

SEQ ID NO: 335 - protein, 102 aa, Fischerella muscicola; identical to SEQ ID NO: 123

SEQ ID NO: 336 - protein, 35 aa, Fischerella muscicola; identical to SEQ ID NO: 120

SEQ ID NO: 337 - protein, 102 aa, Lyngbya aestuarii; identical to SEQ ID NO: 125

SEQ ID NO: 338 - protein, 35 aa, Lyngbya aestuarii; identical to SEQ ID NO: 126

SEQ ID NO: 339 - protein, 102 aa, Leptolyngbya sp.; identical to SEQ ID NO: 129

SEQ ID NO: 340 - protein, 35 aa, Leptolyngbya sp.; identical to SEQ ID NO: 130

SEQ ID NO: 341 - protein, 102 aa, Crocosphaera watsonii; identical to SEQ ID NO: 131

SEQ ID NO: 342 - protein, 35 aa, Crocosphaera watsonii; identical to SEQ ID NO: 132

SEQ ID NO: 343 - protein, 100 aa, Coleofasciculus chthonoplastes; identical to SEQ ID NO: 133

SEQ ID NO: 344 - protein, 35 aa, Coleofasciculus chthonoplastes; identical to SEQ ID NO: 134

SEQ ID NO: 345 - protein, 98 aa, Chroococcidiopsis sp.; identical to SEQ ID NO: 135

SEQ ID NO: 346 - protein, 35 aa, Chroococcidiopsis sp.; identical to SEQ ID NO: 136

SEQ ID NO: 347 - protein, 102 aa, Raphidiopsis brookii; identical to SEQ ID NO: 139

SEQ ID NO: 348 - protein, 35 aa, Raphidiopsis brookii; identical to SEQ ID NO: 140

SEQ ID NO: 349 - protein, 99 aa, Cyanothece sp.; identical to SEQ ID NO: 141

SEQ ID NO: 350 - protein, 35 aa, Cyanothece sp.; identical to SEQ ID NO: 142

SEQ ID NO: 351 - protein, 100 aa, Xenococcus sp.; identical to SEQ ID NO: 143

SEQ ID NO: 352 - protein, 35 aa, Xenococcus sp.; identical to SEQ ID NO: 144

SEQ ID NO: 353 - protein, 100 aa, Pleurocapsa sp.; identical to SEQ ID NO: 145

SEQ ID NO: 354 - protein, 35 aa, Pleurocapsa sp.; identical to SEQ ID NO: 146

SEQ ID NO: 355 - protein, 102 aa, Cylindrospermopsis raciborskii; identical to SEQ ID NO: 147

SEQ ID NO: 356 - protein, 35 aa, Cylindrospermopsis raciborskii; identical to SEQ ID NO: 140

SEQ ID NO: 357 - protein, 100 aa, Microcystis aeruginosa; identical to SEQ ID NO: 79

SEQ ID NO: 358 - protein, 35 aa, Microcystis aeruginosa; identical to SEQ ID NO: 80

SEQ ID NO: 359 - protein, 100 aa, Microcystis aeruginosa; identical to SEQ ID NO: 81

SEQ ID NO: 360 - protein, 35 aa, Microcystis aeruginosa; identical to SEQ ID NO: 82

SEQ ID NO: 361 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 362 - protein, 224 aa, synthetic construct
  source 1..224: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYALSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 363 - protein, 240 aa, synthetic construct
  source 1..240: note = synthetic polypeptide
MVKVIGRRSLGVQRIFDIGLPQDHNFLLANGAIAANCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 364 - protein, 240 aa, synthetic construct
  source 1..240: note = synthetic polypeptide
MIKIATRKYLGKQNVYDIGVERDHNFALKNGFIASNCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 365 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHKFMTTDGQMLAIEEIFARQLDLLTLENIKQTEEALDNHRLPFPLLDAGTIK

SEQ ID NO: 366 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHKFLTTDYQLLAIEEIFARQLDLLTLENIKQTEEALDNHRLPFPLLDAGTIK

SEQ ID NO: 367 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHKFLTTDGQLLAIEEIFARQLDLLTLENIKQTEEALDNHRLPFPLLDAGTIK

SEQ ID NO: 368 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHKFLTTDGQMLAIEEIFARQLDLLTLENIKQTEEALDNHRLPFPLLDAGTIK

SEQ ID NO: 369 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVFEYELEDGSVIRATKDHRFLTTDYQLLPIDEIFARQLDLLTLENIKQTEEALDNHRLPFPLLDAGTIK

SEQ ID NO: 370 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATSDHRFLTTDYQLLPIEEIFARQLDLLTLENIKQTEEALDNHRLPFPLLDAGTIK

SEQ ID NO: 371 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVLEYELEDGSVIRATKDHRFLTTDYQLLPIEEIFARQLDLLTLENIKQTEEALDNHRLPFPLLDAGTIK

SEQ ID NO: 372 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDPEGRVYTQAIAQWHDRGEQEVFEYELEDGSVIRATKDHRFLTTDYQLLPIEEIFARQLDLLTLENIKQTEEALDNHRLPFPLLDAGTIK

SEQ ID NO: 373 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVEKRIECSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 374 - protein, 245 aa, synthetic construct
  source 1..245: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSFGTEILTVEYGPLPIGKIVSEEINCSVYSVDNNGNIYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 375 - protein, 240 aa, synthetic construct
  source 1..240: note = synthetic polypeptide
MIKIATRRSLGVQRIFDIGLPQDHNFLLANGAIAANCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 376 - protein, 240 aa, synthetic construct
  source 1..240: note = synthetic polypeptide
MVKVIGRRSLGVQNVYDIGLPQDHNFLLANGAIAANCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 377 - protein, 240 aa, synthetic construct
  source 1..240: note = synthetic polypeptide
MVKVIGRRSLGVQRIFDIGVERDHNFLLANGAIAANCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 378 - protein, 240 aa, synthetic construct
  source 1..240: note = synthetic polypeptide
MVKVIGRRSLGVQRIFDIGLPQDHNFALKNGFIAANCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 379 - protein, 240 aa, synthetic construct
  source 1..240: note = synthetic polypeptide
MVKVIGRRSLGVQRIFDIGLPQDHNFLLANGAIASNCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 380 - protein, 411 aa, synthetic construct
  source 1..411: note = synthetic polypeptide
TGCCTGTCTTACGACACAGAGATTCTGACCGTTGAATATGGATTCCTTCCTATCGGTAAGATCGTGGAGGAACGGATTGAATGCACAGTCTATACGGTAGATAAAAATGGCTTTGTGTATACACAACCTATTGCTCAGTGGCATAACCGGGGAGAACAGGAAGTTTTCGAATACTGCTTAGAAGACGGTTCGATTATCCGTGCAACGAAAGATCACAAATTTATGACGACCGACGGTCAGATGTTACCGATTGATGAGATTTTCGAACGGGGGTTAGACCTGAAACAAGTTGATGGTTTGCCGATGGTCAAGATCATTAGTCGTAAGAGTCTGGGCACTCAAAACGTCTACGATATTGGAGTAGAAAAAGATCATAATTTTTTGCTGAAGAATGGGCTGGTGGCCTCTAAC

SEQ ID NO: 381 - protein, 223 aa, synthetic construct
  source 1..223: note = synthetic polypeptide
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAEYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 382 - protein, 240 aa, synthetic construct
  source 1..240: note = synthetic polypeptide
MVKIISRKSLGTQNVYDIGVEKDHNFLLKNGLVASNCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 383 - skipped

SEQ ID NO: 384 - protein, 299 aa, synthetic construct
  source 1..299: note = synthetic polypeptide
MEQKLISEEDLSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 385 - protein, 119 aa, synthetic construct
  VAR_SEQ 38: note = X can be F, G, R or E
  source 1..119: note = synthetic polypeptide
MVKIISRKSLGTQNVYDIGVEKDHNFLLKNGLVASNCXNSVVTHGDFSLDNLIFDEGKLIGCIDVGRVGIADRYQDLAILWNCLGEFSPSLQKRLFQKYGIDNPDMNKLQFHLMLDEFF

SEQ ID NO: 386 - protein, 408 aa, synthetic construct
  source 1..408: note = synthetic polypeptide
TGCCTGAGCTACGACACCGAGATCCTGACCGTGGAGTACGGCTTCCTGCCCATCGGCAAGATCGTGGAGGAGCGCATCGAGTGCACCGTGTACACCGTGGACAAGAACGGCTTCGTGTACACCCAGCCCATCGCCCAGTGGCACAACCGCGGCGAGCAGGAGGTGTTCGAGTACTGCCTGGAGGACGGCAGCATCATCCGCGCCACCAAGGACCACAAGTTCATGACCACCGACGGCCAGATGCTGCCCATCGACGAGATCTTCGAGCGCGGCCTGGACCTGAAGCAGGTGGACGGCCTGCCCGTGAAGATCATCAGCCGCAAGAGCCTGGGCACCCAGAACGTGTACGACATCGGCGTGGAGAAGGACCACAACTTCCTGCTGAAGAACGGCCTGGTGGCCAGCAAC

SEQ ID NO: 387 - protein, 580 aa, synthetic construct
  source 1..580: note = synthetic polypeptide
MGWSCIILFLVATATGVHSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSATTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKASGGCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPGHHHHHHG

SEQ ID NO: 388 - protein, 255 aa, synthetic construct
  source 1..255: note = synthetic polypeptide
MGHHHHHHSGVKIISRKSLGTQNVYDIGVEKDHNFLLKNGLVASNCFNSGGCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAHHHHHH

SEQ ID NO: 389 - protein, 35 aa, synthetic construct
  source 1..35: note = synthetic polypeptide
VKIISRKSLGTQNVYDIGVGEPHNFLLKNGLVASN

SEQ ID NO: 390 - protein, 136 aa, synthetic construct
  source 1..136: note = synthetic polypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPVKIISRKSLGTQNVYDIGVEKDHNFLLKNGLVASN

SEQ ID NO: 391 - protein, 136 aa, synthetic construct
  source 1..136: note = synthetic polypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLPVKIISRKSLGTQNVYDIGVGEPHNFLLKNGLVASN

SEQ ID NO: 392 - protein, 137 aa, synthetic construct
  source 1..137: note = synthetic polypeptide
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVERDHNFALKNGFIASN

SEQ ID NO: 393 - protein, 136 aa, synthetic construct
  source 1..136: note = synthetic polypeptide
CLSYDTEILTVEYGFLPIGKIVEERIECNTYSTDKNGFVYTQPIAQWHNRGEQEVFEYCLEDGSIIRATKDHKFMTTDGQMLPIDEIFERGLDLKQVDGLPVKIISRKSLGTQNVYDIGVEKDHNFLLKNGLVASN

SEQ ID NO: 394 - protein, 137 aa, Nostoc punctiforme; identical to SEQ ID NO: 392

SEQ ID NO: 395 - protein, 137 aa, Chroococcidiopsis thermalis
CLSYDTEILTVEYGAIPIGKIVEERIECTVYSVDNNGFIYTQPIAQWHNRGQQEVFEYCLEDGSIIRATKDHKFMTFEGKMLPIDEIFEQELDLKQVKSIQNVKIISRKSLGIQPVYDIGVERDHKFVLKNGLVASN

SEQ ID NO: 396 - protein, 137 aa, Nodularia spumigena
CLSYDTEILTVEYGYIPIGEIVEKAIECSVYSVDNNGNVYTQPIAQWHNRGEQEVFEYSLEDGSTIRATKDHKFMTTDGQMLPIDEIFAQELDLLQVHGLPKVKITARKFVGRENVYDIGVERYHNFAIKNGLIASN

SEQ ID NO: 397 - protein, 137 aa, Anabaena cylindrica
CLSYDTEVLTVEYGFIPIGEIVEKRIECSIFSVDKNGNVYTQPIAQWHNRGRQEIYEYCLDDGSKIRATKDHKFMTTAGEMLPIDEIFERDLDLLKVEGLPEVKIISRQYLGQADVYDIGVEEDHNFAIKNGFIASN

SEQ ID NO: 398 - protein, 137 aa, Calothrix sp.
CLSYDTEVLTVEYGLLPIGEIVEKGIECRVFSVDNHGNVYTQPIAQWHNRGQQEVFEYGLDDGSVIRATKDHKFMTTDGKMLPIDEIFERGLDLLQVQGLPEVKVITRKYIGKENVYDIGVELDHNFAIRNGLVASN

SEQ ID NO: 399 - protein, 137 aa, Nostoc sp.
CLSYDTEILTVEYGFLPIGEIVEKGIECTVFSVASNGIVYTQPIAQWHNRGQQEIFEYCLEDGSIIRATKDHKFMTQDGQMLPIDEIFACELDLLQVQGLPEVKVVTRKYIGKENVYDIGVERDHNFVIRNGLVASN

SEQ ID NO: 400 - protein, 137 aa, Nostoc azollae
CLSYKTEVLTVEYGLIPIGEIVEKRIECSLFSVDENGNIYTQPIAQWHHRGVQEVYEYCLDDGTIIRATKDHKFMTTIGEMLPIDEIFERDLNLLQVNGLPTVKIISRQFLGPANVYDIGVAQDHNFAIKNGLIASN

SEQ ID NO: 401 - protein, 137 aa, Nostoc sp.
CLSYDTEVLTVEYGFVPIGEIVEKGIECSVFSINNNGIVYTQPIAQWHHRGKQEVFEYCLEDGSIIKATKDHKFMTQDGKMLPIDEIFEQELDLLQVKGLPEIKIASRKFLGVENVYDIGVRRDHNFFIKNGLIASN

SEQ ID NO: 402 - protein, 137 aa, Anabaena variabilis
CLSYDTEVLTVEYGFVPIGEIVDKGIECSVFSIDSNGIVYTQPIAQWHHRGKQEVFEYCLEDGSIIKATKDHKFMTQDGKMLPIDEIFEQELDLLQVKGLPEIKIASRKFLGVENVYDIGVGRDHNFFVKNGLIASN

SEQ ID NO: 403 - protein, 135 aa, Pleurocapsa sp.
CLSYDTKILTVEYGAMPIGKIVEEQIDCTVYTVNQNGFVYTQPIAQWHDRGKQEIFEYCLEDGSIIRATKDHKFMTTDGQMLPIDKIFEKGLDLKTINCDVKILSRKSLGIQSVYDIGVEKDHNFLLANGLVASN

SEQ ID NO: 404 - protein, 135 aa, Cyanothece sp.
CLSYETQIMTVEYGLMPIGKIVEEQIDCTVYTVNKNGFVYTQPIAQWHYRGEQEVFEYCLEDGSTIRATKDHKFMTTDGQMLPIDEIFEQGLELKQIHLSVKIISRQSLGIQPVYDIGVEKDHNFLISDGLIASN

SEQ ID NO: 405 - protein, 134 aa, Cyanothece sp.
CLSYDTEILTVEYGPMPIGKIVEEQIECTVYTVDKNGLVYTQPIAQWHHRGQQEVFEYCLEDGSIIRATKDHKFMTDDGQMLPIEEIFEKGLELKQIILVKIISRQLAGNQTVYDLGVEKDHNFLLANGLIASN

SEQ ID NO: 406 - protein, 137 aa, Nostoc sp.
CLSYDTQVLTVEYGLVPIGEIVEKQLECSVFTIDGHGYVYTQAIAQWHNRGQQEVFEYGLEDGSVIRATKDHKFMTTDGQMLPIDEIFERELDLLQVQGLRWVKIITRKYIGQANVYDIGVAQDHNFVIENRLIASN

SEQ ID NO: 407 - protein, 136 aa, Tolypothrix bouteillei
CLSYDTEILTVEYGFLPIGKIVEKGIECNVYSVDKNGNIYTQPIAQWHDRGEQEVFEYCLENGSVIRATKDHKFMTTSGEMLPIDEIFERGLDLIRVEDLPVKILTRKSIGKQTVYDIGVERDHNFVIKNGSVASN

SEQ ID NO: 408 - protein, 136 aa, Aphanizomenon ovalisporum
  VAR_SEQ 87: note = Any amino acid
CLSADTEILTVEYGFLPIGEIVGKAIECRVYSVDGNGNIYTQSIAQWHNRGEQEVFEYTLEDGSIIRATKDHKFMTTDGEMLPIDEXFARQLDLMQVQGLHVKITARKFVGRENVYDIGVEHHHNFAIKNGLIASN

SEQ ID NO: 409 - protein, 137 aa, Oscillatoria nigro-viridis
CLSYDTKILTVEYGPMAIGKIVEEKIECTVYSVDSNGYIYTQSIAQWHRRGQQEVFEYCLEDGSIIRATKDHKFMTVGGQMLPIDEIFEQGLDLKQINSSSDVKIISRKSLGTQEVYDIGVEREHNFILENSLVASN

SEQ ID NO: 410 - protein, 135 aa, Rivularia sp.
CLSYDTEVLTEEFGLIPIGKIVEEKIDCTVYSVDVNGNVYSQPIAQWHNRGMQEVFEYELEDGSTIRATKDHKFMTVDGEMLAIDEIFEKGLELKRVGIYVKIISRKVLKTENVYDIGLEGDHNFIIKDGLIASN

SEQ ID NO: 411 - protein, 137 aa, Trichodesmium erythraeum
CLTYETEIMTVEYGPLPIGKIVEYRIECTVYTVDKNGYIYTQPIAQWHNRGMQEVYEYSLEDGTVIRATPEHKFMTEDGQMLPIDEIFERNLDLKCLGTLELVKIVSRKLAKTENVYDIGVTKDHNFVLANGLIASN

SEQ ID NO: 412 - protein, 137 aa, Microcoleus sp.
CLSYDSEILTVEYGLMPIGKIVEEGIECTVYSVDSHGYLYTQPIAQWHHRGQQEVFEYDLEDGSVIRATKDHKFMTSEGQMLAIDEIFERGLELKQVKRSQPVKIVRRKSLGIQTVYDIGVERDHNFLLANGLVASN

SEQ ID NO: 413 - protein, 137 aa, Stanieria cyanosphaera
CLSYDTEILTVEYGAMPIGKIVKEQIECNVYTVNQNGFIYPQAIAQWHERGKQEIFEYTLDNGLVIRATKDHKFMTIDGQMLPIDEIFERGLELQRINDYSNVKIVSRKSLGKQPVYDIGVTKDHNFLLSNGVVASN

SEQ ID NO: 414 - protein, 137 aa, Calothrix sp.
CLSYDTEILTWEYGFLKIGEIVEKQILCSVFSVDEQGNVYTQPIAQWHNRGLQELFAYQLEDGGVIRATKDHKFMTTDGQMLAIDEIFERQLDLFQVKGLPEVKIISRKVLKTENVYDIGLEGDHNFIIKDGLIASN

SEQ ID NO: 415 - protein, 134 aa, Cyanobacterium stanieri
CLSYDTEVLTVEYGVLPIGKIVEEQIQCTVYSVDQYGFVYTQAIAQWHDRGEQEVFEYELENGATIKATKDHKMMTSDGQMLPIDQIFEQGLDLFMVSFVKIVKRRSHGIQKVYDIGVAKDHNFLLHNGLVASN

SEQ ID NO: 416 - protein, 134 aa, Cyanothece sp.
CLSYDTEILTVEYGPMPIGKIVEENINCTVYTVDPNGFVYTQAIAQWHYRGEQEIFEYYLEDGATIRATKDHKFMTMEGKMLPIDEIFENNLDLKQLTLVKIIGRQSLGVQKVYDIGVEKEHNFLLHNGLIASN

SEQ ID NO: 417 - protein, 134 aa, Cyanothece sp.
CLSYDTEILTVEYGAIPIGKVVEENIDCTVYTVDKNGFVYTQNIAQWHLRGQQEVFEYYLDDGSILRATKDHQFMTLEGEMLPIHEIFERGLELKKIKIVKIVSYRSLGKQFVYDIGVAQDHNFLLANGSIASN

SEQ ID NO: 418 - protein, 136 aa, Anabaena sp.
CLSYDTEILTVEYGFLEIGEIVEKQIECKVYTIDSNGMLYTQSIAQWHNRGQQEVYEYLLENGAIIRATKDHKFMTEAGQMLPIDEIFAQGLDLLQVGVAEVKIVSRTYVGQANVYDIGVESDHNFVIKNGFIASN

SEQ ID NO: 419 - protein, 137 aa, Aphanothece halophytica
CLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEMVKIIKRQSLGRQNVYDVCVETDHNFVLANGCVASN

SEQ ID NO: 420 - protein, 137 aa, Halothece sp.
CLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEMVKIIKRQSLGRQNVYDIGVETDHNFVLANGCVASN

SEQ ID NO: 421 - protein, 137 aa, Cyanobacterium aponinum
CLSYDTEILTVEYGAISIGKIVEEKINCQVYSVDKNGFIYTQNIAQWHDRGSQELFEYELEDGRIIKATKDHKMMTKDGQMLAINDIFEQELELYSVDDMGVVKIVKRRSLGVQPVYDIGVEKDHNFILANGLVASN

SEQ ID NO: 422 - protein, 133 aa, unidentified
  REGION 1..133: note = Description of Unknown: Candidatus Atelocyanobacterium thalassa isolate sequence
CLSYDTKVLTVEYGPLPIGKVVQENIRCRVYTTNDQGLIYTQPIAQWHNRGKQEIFEYHLDDKTIIRATKEHQFMTVDHVMMPIDEIFEQGLELKKIKLKIIRRKSLGMHEVFDIGLEKDHNFVLSNGLIASN

SEQ ID NO: 423 - protein, 137 aa, Oscillatoria limnetica
CLSYNTEVLTVEYGPLPIGKIVDEQIHCRVYSVDENGFVYTQAIAQWHDRGYQEIFAYELADGSVIRATKDHQFMTEDGQMFPIDEIWEKGLDLKKLPTVQDVKIVRRQSLGVQNVYDIGVEKDHNFLLASGEIASN

SEQ ID NO: 424 - protein, 137 aa, unidentified
  source 1..137: note = Cyanobacterium endosymbiont of Epithemia turgida sequence
CLSYDTEVLTVEYGAIPIGRMVEESLDCTVYTVDKNGFVYTQSIQQWHSRGQQEIFEYCFEDGSIIRATKDHKFMTAEGKMSSIHDIFEQGLELKKIIPWSGAKIISCKSLGKQSVYDIGVVQDHNFLLANGVVASN

SEQ ID NO: 425 - protein, 133 aa, Synechococcus sp.
CLGYDTPVLTVEYGFMPIGKIVEEKIQCHVYSVDQNGLVFTQAIAQWHNRGQQEVWEYNLDNGDIVRATKDHKFMTIDGQMLPINQIFEQGLELKVIAVKIVSCKPLRVQTVYDIGVEKDHNFILDNGLVASN

SEQ ID NO: 426 - protein, 134 aa, Dactylococcopsis salina
CLSYDTEVLTEEYGAIPIGKIVEERMNCHVYSVDENGFIYSQPIAQWHPRGEQEVVEYTLEDGKIIRATADHKMMTETGEMLPIEQIFQQQLDLKISNQVKIINRQSLGKQTVYDIGVEKDHNFILGNGLVASN

SEQ ID NO: 427 - protein, 137 aa, Cylindrospermum stagnale
CLSYDTEILTVEYGFIPIGEIVEKRIECSVYSVDNHGNVYTQPIAQWHNRGLQEVFEYCLEDGSTIRATKDHKFMTTDKEMLPIDEIFERGLDLLRVEGLPIVKIIMRSYVGRENVYDIGVERDHNFVAKNGLIAAN

SEQ ID NO: 428 - protein, 137 aa, Synechocystis sp.
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQVKVIGRRSLGVQRIFDIGLPQDHNFLLANGAIAAN

SEQ ID NO: 429 - protein, 137 aa, Geitlerinema sp.
CLSYETPVMTVEYGPLPIGRIVEEQLDCTVYSVDEQGHVYTQPVAQWHHRGLQEVVEYELEDGRRLRATADHRFMTETGEMLPLAEIFERGLELRQVALRVPVKIVSRRSLGMQLVYDIGVAADHNFVLADGLIAAN

SEQ ID NO: 430 - protein, 137 aa, Synechocystis sp.
CLSFDAEILTVEYGPLSIGKIVGEEINCSVYSVDPQGRIYTQAIAQWHDRGVQEVFEYELEDGSVIRATPDHRFLTTDYELLAIEEIFARQMDLLTLTNLKLVKVVRRRSLGMHRVFDIGLAQDHNFLLANGAIAAN

SEQ ID NO: 431 - protein, 135 aa, Microcystis aeruginosa
CLGGETLILTEEYGLLPIAKIVSEEVNCTVYSVDKNGFVYSQPISQWHERGLQEVFEYTLENGQTIQATKDHKFMTNDGEMLAIDTIFERGLDLKSSDFSVKIISRQSLGRKPVYDIGVEKDHNFLLGNGLIASN

SEQ ID NO: 432 - protein, 135 aa, Microcystis aeruginosa
CLGGETLILTEEYGLLPIAKIVSEEINCTVYTVDQNGFVYSQPISQWHERGLQEVFEYTLENGQTIQATKDHKFMTSDGEMLAIDTIFERGLDLKSSDFSVKIIGRQSLGRKPVYDIGVEKDHNFLLGNGLIASN

SEQ ID NO: 433 - protein, 137 aa, Acaryochloris marina
CLSYDTPVLTLEYGWLPIGQVVQEQIECQVFSINERGHLYTQPIAQWHHRGQQEVFEYTLADGSTIQATAEHQFMTTDGQMYPVQQIFEEGLSLKQLPLPWQVKIIQRRSLGLQSVYDIGLAQDHNFVMANGWVAAN

SEQ ID NO: 434 - protein, 137 aa, Leptolyngbya sp.
CLDGETPIVTVEYGVLPIREIVEKELLCSVYSIDENGFVYTQPVEQWHQRGDRQMFEYQLDNGGVIRATPDHKFLTTEGEMVAIDEIFEKGLNLAEFAPADLVKILRRHSIGKAKTYDIGVSKNHNFLLANGLFASN

SEQ ID NO: 435 - protein, 137 aa, Synechococcus elongatus
CLAADTEVLTVEYGPIAIGKLVEENIRCQVYCCNPDGYIYSQPIGQWHQRGEQEVIEYELSDGRIIRATADHRFMTEEGEMLSLDEIFERSLELKQIPTPLLVKIVRRRSLGVQPVYDLGVATVHNFVLANGLVASN

SEQ ID NO: 436 - protein, 137 aa, Synechococcus sp.
CLSADTELYTVEYGWLPIGRLVEEQIECQVLSVNAHGHVYSQPIAQWHRRAWQEVFEYQLETGGTIKATTDHQFLTTDGQMYRIEDIFQRGLDLWQLPPDRFVKIISRCSLGIQPVYDIGVAQDHNFVIRGGLVASN

SEQ ID NO: 437 - protein, 137 aa, Thermosynechococcus elongatus
CLSGETAVMTVEYGAVPIRRLVQERLSCHVYSLDGQGHLYTQPIAQWHFQGFRPVYEYQLEDGSTICATPDHRFMTTRGQMLPIEQIFQEGLELWQVAIAPRGKIVGRRLMGWQAVYDIGLAADHNFVLANGAIAAN

SEQ ID NO: 438 - protein, 137 aa, Thermosynechococcus sp.
CLSGETAVMTVEYGAVPIRRLVQERLTCHVYSLDAQGHLYTQPIAQWHFQGFRPVYEYQLEDGSTIWATPDHRFMTTRGQMLPIEQIFQEGLELWQGPIAPSCKIVGRQLVGWQAVYDIGVARDHNFLLANGAIAAN

SEQ ID NO: 439 - protein, 137 aa, Thermosynechococcus vulcanus
CLSGETAVMTVEYGAIPIRRLVQERLICQVYSLDPQGHLYTQPIAQWHFQGFRPVYAYQLEDGSTICATPDHRFMTTSGQMLPIEQIFREGLELWQVAIAPPCKIVGRRLVGWQAVYDIGLAGDHNFLLANGAIAAN

SEQ ID NO: 440 - protein, 137 aa, Synechococcus sp.
CLAGGTPVVTVEYGVLPIQTIVEQELLCHVYSVDAQGLIYAQLIEQWHQRGDRLLYEYELENGQMIRATPDHRFLTTTGELLPIDEIFTQNLDLAAWAVPDSVKIIRRKFIGHAPTYDIGLSQDHNFLLGQGLIAAN

SEQ ID NO: 441 - protein, 136 aa, Scytonema hofmanni
CLSYDTEVLTAEYGFLPIGKIVEKAIECTVYSVDNDGNIYTQPIAQWHDRGQQEVFEYSLDDGSVIRATKDHKFMTTGGQMLPIDEIFERGLDLMRIDSLPVKILTRKSIGKQTVYDIGVERDHNFVIKNGLVASN

SEQ ID NO: 442 - protein, 136 aa, Westiella intricata
CLSYDTEILTVEYGFLPIGEIVEKRIECTVYTVDTNGYVYTQAIAQWHNRGEQEVFEYALEDGSIIRATKDHKFMTSEGQMLPIDEIFVKGLDLLQVQGLPVKIITRKFLGIQNVYDIGVEQNHNFVIKNGLVASN

SEQ ID NO: 443 - protein, 136 aa, Fischerella sp.
CLSYDTEILTVEYGFLPIGEIVEKGIECTVYTVDNNGNVYTQTIAQWHNRGQQEVFEYCLEDGSVIRATKDHKFMTTDGQMLPIDEIFARGLDLLQVKNLPVKIVTRRPLGTQNVYDIGVESDHNFVIKNGLVASN

SEQ ID NO: 444 - protein, 137 aa, Mastigocladopsis repens
CLSYDTEVLTVEYGFLPIGEIVEKSIECSVYTVDSNGNVYTQPIAQWHNRGQQEVFEYCLEDGSIIRATKDHKFMTIHGQMLPIDEIFERGLELMKIQGLPEAKIITRKSLGTQNVYDIGVERDHNFVTRDGFIASN

SEQ ID NO: 445 - protein, 137 aa, Scytonema hofmanni
CLSYNSEVLTVEYGFLPIGKIVEKGIECSVYSVDSYGKIYTQVIAQWHNRGQQEVFEYCLEDGTIIQATKDHKFMTVDGQMLPIDEIFERGLDLMQVQGLPDVKIITRKSLGTQNVYDIGVSSDHNFVMKNGLIASN

SEQ ID NO: 446 - protein, 137 aa, Anabaena sp.
CLSSDTEVLTVEYGLIPIGEIIEKRIDCSVFSVDKNGNIYTQPIAQWHDRGIQELYEYCLDDGSTIRATKDHKFMTTAGEMLPIDEIFERGLDLLKVHNLPQVKIITRNYVGKENVYDIGVERDHNFAIKNGLIASN

SEQ ID NO: 447 - protein, 137 aa, Fischerella sp.
CLSYDTEVLTVEYGFLPIGEIVEKRIECTVYTVDHNGYVYTQPIAQWHNRGYQEVFEYGLEDGSVIRATKDHKFMTSEGQMLPIDEIFARELDLLQVTGLVNVKIVTRRLLGIQNVYDIGVEQNHNFVIKNGLVASN

SEQ ID NO: 448 - protein, 137 aa, Calothrix sp.
CLSYDTEIFTVEYGFLPIGEIVEKRLECTVLTVDNHGNIYSQPIAQWHHRGQQQIYEYGLEDGSVIRATKDHKFMTTDGQMLPIDEIFERGLDLLQVTNLDNVKVITRKLADTENVYDIGVENHHNFLIKNGLVASN

SEQ ID NO: 449 - protein, 136 aa, Fischerella thermalis
CLSYETEILTVEYGFLPIGEIVEKRIECSVYTVDNNGYVCTQPIAQWHNRGYQEVFEYGLEDGSVIRATKDHKFMTIDRQMLPIDEIFARGLDLLQVTGLPVKIITRKSLGTQNVYDIGVEQNHNFVIKNGLVASN

SEQ ID NO: 450 - protein, 137 aa, unidentified
  source 1..137: note = cyanobacterium PCC 7702 Chl7702 sequence
CLSYDTEILTVEYGFLSIGEIVEKEIECTVYTVDSNGYIYTQPIAQWHEQGEQEIFEYSLEDGSTIRATKDHKFMTIEGEMLPIDQIFARQLDLMQITGLPQVKISTKKSLGKQKVYDIGVVRDHNFIIKNGFVASN

SEQ ID NO: 451 - protein, 136 aa, Fischerella sp.
CLSYDTEVLTVEYGFLPIGEIVEKRIECTVYTVDTNGYVYTQAIAQWHNRDEQEVFEYALEDGSIIRATKDHKFMTSEGQMLPIDEIFAKGLDLLQVQGLPVKIVTRKFLGIQNVYDIGVEQNHNFVIKNGLVASN

SEQ ID NO: 452 - protein, 137 aa, Fischerella muscicola
CLSYETEILTVEYGFLPIGEIVEKRIECSVYTVDNNGYVCTQTIAQWHNRGYQEVFEYGLEDGSVIRATKDHKFMTIDRQMLPIDEIFARGLDLLQVKGLPEVKIITRQSLGTQNVYDIGVEQNHNFVIKNGLVASN

SEQ ID NO: 453 - protein, 137 aa, Fischerella muscicola
CLSYDTEVLTVEYGFLPIGEIVEKTIECNVFTVDSNGYVYTQPIAQWHNRGYQEVFEYGLEDGSVIRATKDHKFMTSEGKMLPIDEIFARELDLLQVTGLINVKIVTRKFLGIQNVYDIGVEQNHNFVIKNGLVASN

SEQ ID NO: 454 - protein, 137 aa, Lyngbya aestuarii
CLSYDTEILTVEYGAIPIGKVVDEKIECTVYSVDKNGLIYTQPIAQWHNRGKQEVFEYSLEDGSTIRATKDHKFMTMDNQMLPIDEILEKGLELKQVNADSVVKIVSRKSLDSQTVYDIGVETDHNFLLANGSVASN

SEQ ID NO: 455 - protein, 135 aa, Microchaete sp.
CLSYKTQVLTVEYGLLAIGEIVEKNIECSVFSVDIHGNVYTQPIAQWHHRGQQEVFEYGLEDGSIIRATKDHKFMTTQGEMLPIDEIFARGLDLLQVKGVVKIITRKYIGKENVYDIGVEQDHNFAIKNGLIAAN

SEQ ID NO: 456 - protein, 137 aa, Leptolyngbya sp.
CLSYDTEILTVEYGALPIGKIVENQMICSVYSIDNNGYIYIQPIAQWHNRGQQEVFEYILEDGSIIRSTKDHKFMTKGGEMLPIDEIFERGLELAQVTRLEQVKIISRRSVGVQSVYDIGVKQDHNFFLRNGLIASN

SEQ ID NO: 457 - protein, 137 aa, Crocosphaera watsonii
CLSYDTEILTVEYGAMYIGKIVEENINCTVYTVDKNGFVYTQTIAQWHNRGEQEIFEYDLEDGSKIKATKDHKFMTIDGEMLPIDEIFEKNLDLKQVVSHPDVKIIGCRSLGTQKVYDIGVEKDHNFLLANGSIASN

SEQ ID NO: 458 - protein, 135 aa, Coleofasciculus chthonoplastes
CLSYDTQILTVEYGAVAIGEIVEKQIECTVYSVDENGYVYTQPIAQWHNRGEQEVFEYLLEDGATIRATKDHKFMTDEDQMLPIDQIFEQGLELKQVEVLVKIIGRKPLGTQPVYDIGVERDHNFLLFNGSVASN

SEQ ID NO: 459 - protein, 133 aa, Chroococcidiopsis sp.
CLSYDTEVLTVEYGAIPIGKIVEEKIACNVYSVDKNGFVYTQPIAQYHDRGIQEVFEYRLENGSVIRATKDHKMMTADGQMLPIDEIFKQNLDLKQLNVKIISRQSLGKQSVFDIGVAKDHNFLLANGLVASN

SEQ ID NO: 460 - protein, 132 aa, Aphanizomenon flos-aquae
CLSYDTEILTVEYGFLQIGEIVEKQIECKVYTVDSNGILYTQSIAQWHNRGQQEVYEYLLENGAIIRATKDHKFMTEEGQMLPIDEIFSQGLDLLQVVKIISRTYVGQANVYDIGVENDHNFVIKNGFIAAN

SEQ ID NO: 461 - protein, 137 aa, Raphidiopsis brookii
CLSYETEVLTLEYGFLPIGEIVDKQMVCTVFSVNDSGNVYTQPIGQWHDRGVQELYEYCLDDGSTIRATKDHKFMTTQGEMVPIDEIFHQGWELVQVSGTMNVKIVSRRYLGKADVYDIGVAKDHNFIIKNGLVASN

SEQ ID NO: 462 - protein, 134 aa, Cyanothece sp.
CLSYDTEILTVEYGPMPIGKIVEENINCSVYTVNKNGFVYTQSIAQWHHRGEQEVFEYYLEDGETIRATKDHKFMTTEGKMLPIDEIFENNLDLKKLTVVKIIERRSLGKQNVYDIGVEKDHNFLLSNNLIASN

SEQ ID NO: 463 - protein, 135 aa, Xenococcus sp.
CLSADTEVLTVEYGAISIGKIVEERIECTVYSVDANGFVYTQEIAQWHNRGEQEVFEYMLDDGSVIRATKDHKLMTIDGQMVAIDEIFSQGLELKQVLGLVKIVSRKSLGTQTVYDLGVARDHNFLLANGTVASN

SEQ ID NO: 464 - protein, 135 aa, Pleurocapsa sp.
CLSYDTEIYTVEYGALPIGKIVESRIKCTVLTVDKNGLVYSQPIVQWHDRGIQEVFEYTLDNGATIRATKDHKFMTVEGQMLPIDEIFELGLELKEIQQFVKIISRQSLGKQSVYDIGVAKDHNFLLANGMVASN

SEQ ID NO: 465 - protein, 137 aa, Cylindrospermopsis raciborskii
CLSYETEVLTLEYGFVPIGEIVNKQMVCTVFSLNDSGNVYTQPIGQWHDRGVQDLYEYCLDDGSTIRATKDHKFMTTQGEMVPIDEIFHQGWELVQVSGISKVKIVSRRYLGKADVYDIGVAKDHNFIIKNGLVASN

SEQ ID NO: 466 - protein, 129 aa, Spirulina major
CLTYDTLVLTVEYGPVPIGKLVEAQINCQVYSVDANGFIYTQAIAQWHDRGQRQVYEYTLEDGSTIRATPDHKFMTATGEMLPIDQIFEQGLDLVKIIHRRALPPQSVYDIGVERDHNFLLPSGWVASN

SEQ ID NO: 467 - protein, 131 aa, Spirulina subsalsa
CLSYDTKIITVEYGAIAIGTIVEQGLHCHVYSVDPNGFIYTQPIAQWHQRGEQEVFAYTLENGSIIQATKDHKFMTQQGKMLPIDTIFEQGLDLLQVKIIKRTSLGVRPVYDIGVIQDHNFLLENGLVASN

SEQ ID NO: 468 - protein, 135 aa, Microcystis aeruginosa
CLGGETLILTEEYGLLPIAKIVSEEINCTVYSVDKNGFIYSQPISQWHERGLQEVFEYTLENGQTIQATKDHKFMTSDGEMLAIDTIFERGLDLKSSDFSVKIISRQFLGRKPVYDIGVEKDHNFLLGNGLIASN

SEQ ID NO: 469 - protein, 130 aa, Myxosarcina sp.
CLSYDTEVLTLKYGALPIGEIVEKRINCHVYTRAESGFFYIQSIEQWHDRGEQEVFEYTLENGATIKATKDHKFMTSGGQMLPIDEIFERGLDLLVKIVSRKSLGKQPVYDLGVAKDHNFLLANGTVASN

SEQ ID NO: 470 - protein, 136 aa, Leptolyngbya sp.
CLSADTQLLTVEYGPLEIGRIVEEQIACHVYSVDANGFVYTQPIAQWHSRGEQEIFEYQLEDGRTLRATADHKFMTTTGEMGRINDIFEQGLDLKQIDLPQVKVVSRQSLGVQPVYDIGVATDHNFLLADGLVASN

SEQ ID NO: 471 - protein, 132 aa, Acaryochloris sp.
CLSYDTPVLTLEYGWLPIGQVVQEQIECQVFSINERGHLYTQPIAQWHHRGQQEVFEYTLTDGSTIQATAEHQFMTTDGQMYPIQQIFEEGLSLKQLVKITQRRSLGLQSVYDIGLAQDHNFVIANGWVAAN

SEQ ID NO: 472 - protein, 133 aa, Geminocystis herdmanii
CLSYDTEVLTVEFGAIPMGKIVEERLNCQVYSVDKNGFIYTQNIAQWHDRGVQEVFEYELEDGRIIKATKDHKMMIENCEMVEIDRIFEEGLELFEVNVKILKRRSISSQQVYDIGVEKDHNFLLANGLVASN

SEQ ID NO: 473 - protein, 133 aa, Nodosilinea nodulosa
CLSADTELLTLEYGPLTIGEIVAKRIPCHVFSVDESGYVYTQPVAQWHQRGHQEVFEYQLDDGTTIRATADHQFMTELGEMMAIDEIFQRGLELKQVEVKIISRQSLGVQPVYDIGVARDHNFLLADGQVASN

SEQ ID NO: 474 - protein, 137 aa, Rubidibacter lacunae
CLSYDTEVLTVEYGPLAIGTIVSERLACTVYTVDRSGFLYAQAISQWHERGRQDVFEYALDNGMTIRATKDHKLMTADGQMVAIDDIFTQGLTLKAIDTAAFMKIVSRKSLGVQHVYDIGVARDHNFLLANGAIASN

SEQ ID NO: 475 - protein, 136 aa, Chlorogloeopsisfritschii
CLSYDTAILTVEYGFLPIGEIVEKGIECTVYTVDSNGYIYTQPIAQWHNRGEQELFEYSLEDGSIIRATKDHKFMTIDGQMLPIDEIFARKLELMQVKGLPVKIIAKKSLGTQNVYDIGVERDHNFVIKNGLVASN

SEQ ID NO: 476 - protein, 137 aa, Richelia intracellularis
CLSYDTQILTVEHGPMSIGEIVEKCLECHVYTVNKNGNICIQTITQWHFRGEQEIFEYELEDGSFIQATKDHKFMTTTGEMLPIHEIFTNGLEILQLSKSLLVKILARKSLGTQKVYDIGVNDDHNFALSNSFIASN

SEQ ID NO: 477 - protein, 137 aa, Synechococcus sp.
CLAGDTPVVTVEYGVLPIQTIVEQELLCQVYSVDAQGLIYTQPIEQWHNRGDRLLYEYELENGQMIRATPDHKFLTTTGELLPIDEIFTQNLDLAAWAVPDSVKIIRRKFIGHAPTYDIGLSQDHNFLLGQGLIAAN

SEQ ID NO: 478 - protein, 137 aa, Synechococcus sp.
CLAGDTPVVTVEYGVLPIQTIVEQELLCHVYSVDAQGLIYTQPIEQWHQRGDRFLYEYELENGQMIRATPDHKFLTTTGKLLPIDEIFTQNLDLAAWAVPDSVKIIRRKFIGHAPTYDIGLSQDHNFLLGQGFIAAN

SEQ ID NO: 479 - protein, 137 aa, Synechococcus sp.
CLAGDTPVVTVEYGVLPIQTIVEQELLCHVYSVDAQGLIYTQPIEQWHQRGDRLLYEYELENGQMIRATPDHKFLTTTGELLPIDEIFTQNLDLAAWAVPDSVKILRRKFIGRAPTYDIGLSQDHNFLLGQGLVAAN

SEQ ID NO: 480 - protein, 129 aa, Synechococcus sp.
CLAGDTPVVTVEYGVLPIRTIVDQELLCHVYSLDPQGFIYAQPVEQWHRRGDRLLYEYELETGAVIRATPDHKFLTATGEMLPIDEIFVRNLDLVKIIRRNLIGEAATYDIGLGKDHNFLLGQGLIASN

SEQ ID NO: 481 - protein, 130 aa, Synechococcus sp.
CLAGGTPVVTVEYGVLPIQTIVEQELLCHVYSVDAQGLIYTQPIEQWHQRGDRLLYEYELENGQMIRATPDHKFLTTTGELLPIDEIFTQNLDLLVKIIRRKFIGHAPTYDIGLSQDHNFLLGQGLIAAN

SEQ ID NO: 482 - protein, 130 aa, Synechococcus sp.
CLAGDTPVVTVEYGVLPIQTIVEQELLCHVYSVDAQGLIYTQPIEQWHKRGDRLLYEYELENGQIIRATPDHKFLTTTGEMRPIDEIFAKNLSLLVKIIRRKFVGHAPTYDIGLSQDHNFLLGQGLIAAN

SEQ ID NO: 483 - protein, 134 aa, Cyanothece sp.; identical to SEQ ID NO: 417

SEQ ID NO: 484 - protein, 137 aa, Synechococcus elongatus; identical to SEQ ID NO: 435

SEQ ID NO: 485 - protein, 137 aa, Chlorogloeopsis fritschii
CLSYDTAILTVEYGFLPIGEIVEKGIECTVYTVDSNGYIYTQPIAQWHNRGEQELFEYSLEDGSIIRATKDHKFMTIDGQMLPIDEIFARKLELMQVKGLPEVKIIAKKSLGTQNVYDIGVERDHNFVIKNGLVASN

SEQ ID NO: 486 - protein, 132 aa, Cyanothece sp.
CLSYDTEILTVEYGPMPIGKIVEENINCTVYTVDPNGFVYTQAIAQWHYRGEQEIFEYYLEDGATIRATKDHKFMTMEGKMLPIDEIFENNLDLKQLVKIIGRQSLGVQKVYDIGVEKEHNFLLHNGLIASN

SEQ ID NO: 487 - protein, 132 aa, Lyngbya majuscula
CLSYDTEIITVEYGPIAIGEIVEKGIPCTVYSVDSNGYVYTQPIAQWHNRGEQEVFEYTLDDGSVIRATKDHKFMTIDGQMLPIDEIFEGGLELKQLVKIISRKSLGTQPVYDIGVKDDHNFILANGMVASN

SEQ ID NO: 488 - protein, 137 aa, unidentified
  source 1..137: note = filamentous cyanobacterium ESFC-1 sequence
CLSYDTEVLTVEYGAVPIGKLVEEKLNCSVYTVDPNGYIYTQAIAQWHDRGIQEVFEYQLEDNTIIRATKDHKFMTEDHQMLPIDEIFERGLELKKCPQPQQVKIIRRRSLGFQPVYDIGLEQDHNFLLNQGAIASN

SEQ ID NO: 489 - protein, 129 aa, Synechococcus sp.
CLAGGTPVVTVEYGVLPIQTIVEQELLCHVYSVDAQGLIYAQLIEQWHQRGDRLLYEYELENGQMIRATPDHRFLTTTGELLPIDEIFTQNLDLVKIIRRKFIGHAPTYDIGLSQDHNFLLGQGLIAAN

SEQ ID NO: 490 - protein, 132 aa, Acaryochloris marina
CLSYDTPVLTLEYGWLPIGQVVQEQIECQVFSINERGHLYTQPIAQWHHRGQQEVFEYTLADGSTIQATAEHQFMTTDGQMYPVQQIFEEGLSLKQLVKIIQRRSLGLQSVYDIGLAQDHNFVMANGWVAAN

SEQ ID NO: 491 - protein, 129 aa, Microcystis aeruginosa
CLGGETLILTEEYGLLPIAKIVSEEVNCTVYSVDKNGFVYSQPISQWHERGLQEVFEYTLENGQTIQATKDHKFMTNDGEMLAIDTIFERGLDLVKIISRQSLGRKPVYDIGVEKDHNFLLGNGLIASN

SEQ ID NO: 492 - protein, 125 aa, Anabaena circinalis
CLSYDTEILTVEYGFLEIGEIVEKQIECKVYTVDSNGILYTQPIAQWHHRGQQEVYEYLLENGAIIRATKDHKFMTEAGEMLPIDDIFTQVKIISRTYVGQANVYDIGVENDHNFVIKNGFVAAN

SEQ ID NO: 493 - protein, 125 aa, Anabaena circinalis
CLSYDTEILTVEYGFLEIGEIVEKQIECRVYTVDSNGILYTQPIAQWHYRGQQEVYEYLLENGAIIRATKDHNFMTEAGEMLPIDDIFTQIKIISRKYVGQANVYDIGVENDHNFVIKNGFVAAN

SEQ ID NO: 494 - protein, 124 aa, Cyanobacterium sp.
CLSYDTKVLTVEYGPLPIGKVVQENIRCRVYTTNDQGLIYTQPIAQWHNRGKQEIFEYHLDDKTIIRATKEHQFMTVDHVMMPIDEIFEQKIIRRKSLGMHEVFDIGLEKDHNFVLSNGLIASN

SEQ ID NO: 495 - protein, 129 aa, Planktothrix sp.
CLSYDTEVLTVEYGLIPISKIVEEKIECTVYTVNNQGYVYTQPIAQWHNRGEQEVFEYYLEDGSVIRATKDHKFMTVEGQMLPIDEIFEKELDLVKIISRKSLGTQPVYDIGVQEDHNFVLNNGLVASN

SEQ ID NO: 496 - protein, 129 aa, Planktothrix sp.
CLSYDTEILTVEYGLMPIGKIVKEKIECTVYTVNNQGYVYTQPIAQWHHRGEQEVFEYCLEDGSVIRATKDHKFMTVQGQMLPIDEIFEKELDLVKIISRKSLGTQPVYDIGVQEDHNFLLNNGLVASN

SEQ ID NO: 497 - protein, 137 aa, Fremyella diplosiphon
CLSYDTEVLTVEYGLIPIGEIVEKRLECSVYSVDINGNVYTQPIAQWHHRGQQEVFEYALEDGSIIRATKDHKFMTTDGQMLPIDEIFERGLDLLQVPHLPEVKIVTRRAIGAANVYDIGVEQDHNFAIKNGLIAAN

SEQ ID NO: 498 - protein, 129 aa, Planktothrix sp.
CLSYDTEILTVEYGLIPISKIVEEKIECTVYTVNNQGYVYTQPIAQWHNRGEQEVFEYYLEDGSVIRATKDHKFMTVDGQMLPIDEIFEKELDLVKIISRKSLGTQPVYDIGVQEDHNFVLNNGLVASN

SEQ ID NO: 499 - protein, 6 aa, synthetic construct
  source 1..6: note = Synthetic 6xHis tag
HHHHHH